SEQUENCE LISTING (1 sequence)

SEQ ID NO: 1 - DNA, 53 bp, synthetic construct
  source 1..53: mol_type = other DNA
catgtaatgcacgtactttcagggtaaacatgtaatgcacgtactttcagggt

(19) United States
(12) Patent Application Publication
(10) Pub. No.: US 2025/0285709 A1
(43) Pub. Date: Sep. 11, 2025

(54) PHASING AND PREPHASING CORRECTION OF BASE CALLING IN NEXT GENERATION SEQUENCING

(71) Applicant: Element Biosciences, Inc., San Diego, CA (US)

(72) Inventors: Minghao GUO, San Diego, CA (US); Rui MA, San Diego, CA (US); Chiung-Ting WU, San Diego, CA (US); Semyon KRUGLYAK, San Diego, CA (US); Ryan KELLEY, San Diego, CA (US); Connor THOMPSON, San Diego, CA (US)

(21) Appl. No.: 18/959,114

(22) Filed: Nov. 25, 2024

Related U.S. Application Data
(63) Continuation of application No. PCT/US2023/023604, filed on May 25, 2023.
(60) Provisional application No. 63/346,256, filed on May 26, 2022.
(60) Provisional application No. 63/413,864, filed on Oct. 6, 2022.

(51) Int. Cl. G16B 30/00; G06T 5/77

(52) U.S. Cl. CPC G16B 30/00; G06T 5/77; G06T 2207/30072

(57) ABSTRACT

The present disclosure includes methods, systems, and media for performing a phasing and prephasing correction in sequencing analysis, comprising: determining corrected image intensities of a plurality of polonies, Ipc(N), based on a cycle N−1 phasing coefficient, pN−1, a cycle N−1 prephasing coefficient, ppN−1, or both; obtaining base calls in the cycle N based on the corrected image intensities of the plurality of polonies in the cycle N, Ipc(N); selecting, by the processor, polonies from the plurality of polonies based on the base calls; determining a cycle N phasing coefficient, pN, a cycle N prephasing coefficient, ppN, or both; and updating image intensities of the plurality of polonies in cycle N, I(N), using updated and corrected image intensities, Ipc_n(N), wherein Ipc_n(N) is obtained based on the cycle N phasing coefficient, pN, the cycle N prephasing coefficient, ppN, or both.

### CROSS REFERENCE TO RELATED APPLICATIONS

This application is a continuation of PCT/US2023/023604 filed May 25, 2023, which claims the benefit of U.S. Provisional Patent Application Ser. Nos. 63/346,256, filed May 26, 2022, and 63/413,864, filed Oct. 6, 2022, which are hereby incorporated by reference in their entireties.

### TECHNICAL FIELD

This disclosure relates generally to correcting unsynchronized sequencing signals, and particularly to phase and prephasing corrections for making accurate base-calling in a digital image of a flow cell during DNA sequencing.

### BACKGROUND

Next generation sequencing-by-synthesis using a flow cell may be used for identifying sequences of DNA. As single-stranded DNA fragments from a sequencing library are flooded across a flow cell, the fragments may attach to the surface of the flow cell. An amplification process is then performed on the DNA fragments, such that copies of a given fragment form a cluster or polony of nucleotide strands. A single cluster may attach to the flow cell at random locations.

In next-generation sequencing (NGS) or NGS-like applications such as sequencing by synthesis, sequencing by binding, or sequencing by avidity, in order to identify the sequence of a target nucleic acid, a new strand is synthesized one nucleotide base at a time. During each cycle, 3′-blocked nucleotides attach at complementary positions on the strands, ensuring that only one base will attach to any given strand during a single cycle. Often, such as in sequencing by synthesis, the blocked nucleotide may also be fluorescently labeled, while in others, such as in sequencing by binding or sequencing by avidity, a label is reversibly or noncovalently bound to the synthesis complex in a separate step that takes place after the blocked nucleotide has been incorporated. During the detection step, the flow cell is exposed to excitation light, exciting the labels and causing them to fluoresce. Because, in most existing techniques, the strands undergoing sequencing are clustered together, the fluorescent signal for any one fragment is amplified by the signal from its clonal counterparts, such that the fluorescence for an entire colony may be recorded by an imager. To initiate subsequent sequencing steps, the blocking groups are then cleaved, the surface is washed, and the cycle repeats. Importantly, at the imaging step of each sequencing cycle, one or more images are recorded. A base-calling algorithm is applied to the recorded images to “read” the successive signals from each cluster or polony and convert the optical signals into an identification of the nucleotide base sequence added to each fragment. However, a cluster or polony may include many copies of a DNA fragment. The sequencing of a particular copy may fall behind other copies within the cluster or polony by one or more residues (phasing) or get ahead of other copies within the cluster or polony by one or more residues (prephasing). Phasing and prephasing may accumulate over cycles, eventually degrading the signal from a given cluster or polony to the point at which the accuracy of base calling is reduced. The phasing and prephasing correction seeks to estimate the amount of signal coming in from the previous and subsequent cycles and remove them. However, it remains a challenge to accurately perform phasing and prephasing correction.

### BRIEF SUMMARY

Provided herein are system, apparatus, method, and/or computer program product aspects, and/or combinations and sub-combinations thereof which enables phasing and prephasing correction of image intensities in multiple polonies or clusters of signals. The image intensities in such polonies or clusters may come from different imaging and/or sequencing methods.

As a particular application of such, aspects of methods, systems, and media for phasing and prephasing correction of image intensities, so that the corrected image intensities may be relied on for accurate base calling.

Other aspects include corresponding computer systems, apparatus, and computer program products recorded on computer storage device(s), which, alone or in combination, configured to perform the actions of the methods. For a computer system configured or to be configured to perform operations or actions, the computer system has installed on it software, firmware, hardware, or their combinations that in operation cause the computer system to perform the operations or actions. For a computer program product configured or to be configured to perform operations or actions, the computer program product includes instructions that, when executed, by a hardware processor, cause the hardware processor to perform the operations or actions.

Further aspects, features, and advantages of the present disclosure, as well as the structure and operation of the various aspects of the present disclosure, are described in detail below with reference to the accompanying drawings.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the art(s) to make and use the aspects.

FIG. 1 illustrates a block diagram of a system for performing phasing and prephasing corrections, according to some aspects.

FIG. 2 is a scatter plot illustrating image intensities of polonies in cycles N and N−1 with phasing and prephasing, according to some aspects.

FIG. 3 is a scatter plot illustrating image intensities of polonies in cycles N and N−1, in FIG. 2, after phasing and prephasing corrections using the technologies herein, according to some aspects.

FIG. 4 illustrates a block diagram of a computer system for phasing and prephasing corrections, according to some aspects.

FIG. 5 illustrates a flow chart of a method for performing phasing and prephasing corrections with two stages, according to some aspects.

FIGS. 6A-6B show flow charts of a method for performing phasing and prephasing corrections with iteration(s), according to some aspects.

FIG. 7 is a schematic showing an exemplary linear single stranded library molecule, according to some aspects.

FIG. 8 is a schematic showing an exemplary linear single stranded library molecule, according to some aspects.

FIG. 9 is a schematic of various exemplary configurations of multivalent molecules, according to some aspects.

FIG. 10 is a schematic of an exemplary multivalent molecule comprising a generic core attached to a plurality of nucleotide-arms, according to some aspects.

FIG. 11 is a schematic of an exemplary multivalent molecule comprising a dendrimer core attached to a plurality of nucleotide-arms, according to some aspects.

FIG. 12 shows a schematic of an exemplary multivalent molecule comprising a core attached to a plurality of nucleotide-arms, where the nucleotide arms comprise biotin, spacer, linker and a nucleotide unit, according to some aspects.

FIG. 13 is a schematic of an exemplary nucleotide-arm comprising a core attachment moiety, spacer, linker and nucleotide unit, according to some aspects.

FIG. 14 shows the chemical structure of an exemplary spacer (top), and the chemical structures of various exemplary linkers, including an 11-atom Linker, 16-atom Linker, 23-atom Linker and an N3 Linker (bottom), according to some aspects.

FIG. 15 shows the chemical structures of various exemplary linkers, including Linkers 1-9, according to some aspects.

FIG. 16 shows the chemical structures of various exemplary linkers joined/attached to nucleotide units, according to some aspects.

FIG. 17 shows the chemical structures of various exemplary linkers joined/attached to nucleotide units, according to some aspects.

FIG. 18 shows the chemical structures of various exemplary linkers joined/attached to nucleotide units, according to some aspects.

FIG. 19 shows the chemical structures of various exemplary linkers joined/attached to nucleotide units, according to some aspects.

FIG. 20 shows the chemical structure of an exemplary biotinylated nucleotide-arm, according to some aspects.

FIG. 21 provides a schematic illustration of one embodiment of the low binding solid supports of the present disclosure, according to some aspects.

FIG. 22 shows an exemplary support with multiple tiles for immobilized polonies or clusters, according to some aspects.

FIG. 23 shows a flow chart of a method for performing phasing and prephasing corrections using a penalty function, according to some aspects.

FIG. 24 shows a comparison of error rate in base calling from a high diversity data set and a low diversity data set using the phasing and prephasing correction methods disclosed herein, according to some aspects.

FIG. 25 is a schematic showing exemplary embodiments of padlock probes, according to some aspects.

FIG. 26 is a schematic showing a workflow for generating inside a cell circularized padlock probes, comprising generating first and second cDNAs from first and second target RNA molecules (respectively), hybridizing first and second padlock probes to the first and second cDNA molecules (respectively) to generate first and second circularized padlock probes (respectively), according to some aspects.

FIG. 27 is a schematic showing a rolling circle and sequencing workflow inside a cell, comprising generating first and second concatemers by conducting rolling circle amplification using first and second covalently closed circular molecules (respectively), according to some aspects.

FIG. 28 is a schematic showing an exemplary workflow for sequencing a concatemer that is generated inside the cell, according to some aspects.

FIG. 29 is a schematic showing an exemplary workflow for sequencing a concatemer that is generated inside the cell, according to some aspects.

FIG. 30 is a schematic showing an exemplary workflow for sequencing a concatemer that is generated inside the cell, according to some aspects.

FIG. 31 is a schematic showing an exemplary workflow for sequencing a concatemer that is generated inside the cell, according to some aspects.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, method, and/or computer program product aspects, and/or combinations and sub-combinations thereof which enables phasing and/or prephasing corrections for accurate base calling in sequencing data analysis. The phasing and/or prephasing correction techniques may be used on image intensities from polonies or clusters obtained from various imaging and/or sequencing techniques. The techniques disclosed herein are useful for base calling in next generation sequencing (NGS), and base-calling will be used as the primary example herein for describing the application of these techniques. However, such image analysis techniques may also be useful in other applications where spot-detection and/or charged coupled device (CCD) imaging is used.

In DNA sequencing, primary analysis can include image processing steps including but not limited to identifying the centers of clusters or polonies and generating base calls of clusters or polonies. Primary analysis can also involve the formation of a template for the flow cell. The template can include the estimated locations of all detected clusters or polonies in a common coordinate system. Templates can be generated by identifying cluster or polony locations in all images (e.g., flow cell images) in the first few sequencing cycles of the sequencing process. The images may be aligned across all the sequencing cycles and/or color channels in the common coordinate system. Cluster or polony locations from different images may be merged based on proximity in the common coordinate system.

After the actual cluster or polony centers are identified in each flow cell image, base calling may be performed based on the actual clusters or polony centers. Each cluster or polony of signals may be used to generate a single base call in one sequencing cycle. Ideally, during each sequencing cycle, all copies, in a polony, of a same nucleotide base or otherwise repetitions of the nucleotide base, in a cluster, which provide amplification of the signal are sequenced synchronously. However, the sequencing of a particular copy may fall behind (phasing) or get ahead (prephasing) than other copies or repetition. Phasing and prephasing may accumulate over sequencing cycles. As such, a cluster or polony of signals may be effected by phasing and prephasing, which may consequently deteriorate accuracy of base calling using such signals. The techniques disclosed herein may be used for phasing and prephasing corrections of image signals. A variety of algorithms exist for phasing and/or prephasing corrections. These existing algorithms suffer from various shortcomings. For example, correction using iterative optimization requires additional computational burden on the processing hardware and causes undesired delay in the estimation and downstream operations. Additionally, the accuracy and reliability of existing algorithms need improvements when the sequencing data is of low or unbalanced diversity, e.g., one or more types of nucleotide bases are each or in total less than 10% of the total bases in a cycle, either separately, or in total.

In some embodiments, the techniques disclosed herein advantageously utilize phasing and prephasing information from a previous and/or a subsequent sequencing cycle to start the correction process for a current sequencing cycle. Such information may provide a reasonable starting point for determining the phasing and prephasing information of the current cycle and achieve correction in a simple and efficient two-stage correction process. The two-stage correction process advantageously saves computation time and reduces computational complexity than existing methods that estimates phasing and/or prephasing correction by optimization of a cost function while achieving accurate and reliable corrections. The techniques disclosed herein also advantageously provide phasing and prephasing correction not only across multiple channels but also within a single channel. That way, the technologies allow each channel to have phasing and prephasing corrections independent of other channels. Further, the techniques disclosed herein are capable of handling correction of low diversity sequencing data, e.g., when one or more bases included in the polonies or clusters are less than 10%, 5%, or even 2% of the total amount of bases. Furthermore, the techniques disclosed herein use a selected subset of polonies for determining the phasing and phasing coefficient which excludes signals that may cause errors or inaccuracy in the estimation of the coefficients.

In some embodiments, the techniques disclosed herein advantageously utilize penalty function(s) in selecting phasing and prephasing coefficients for correction so that the techniques herein can provide improved accuracy and reliability in base calling for samples with low or unbalanced diversity which is comparable to the accuracy and reliability for samples of high or balanced diversity.

In some embodiments, the techniques disclosed herein can be advantageously utilized for phasing and prephasing corrections not only in sequencing samples that are in two-dimensions (2D) but also in sequencing samples that are in situ or otherwise in three-dimensions (3D).

In some embodiments, the techniques herein advantageously allow phasing and prephasing corrections of a specific cycle to be completed within a time window that is not greater than the time window needed to complete the sequencing reactions of one or a couple of cycles so that it advantageously facilitates performing accurate and reliable base calling in parallel with performing sequencing reactions in cycles subsequent to the specific cycle.

In some embodiments, the techniques herein can be advantageously utilized for phasing and prephasing corrections of sample(s) in which the phased or prephased polonies or clusters are above specific densities (e.g., greater than 10⁴ per mm²) and/or percentage (e.g., more than 20%, 30%, or 40%) of a total number of polonies immobilized on a support.

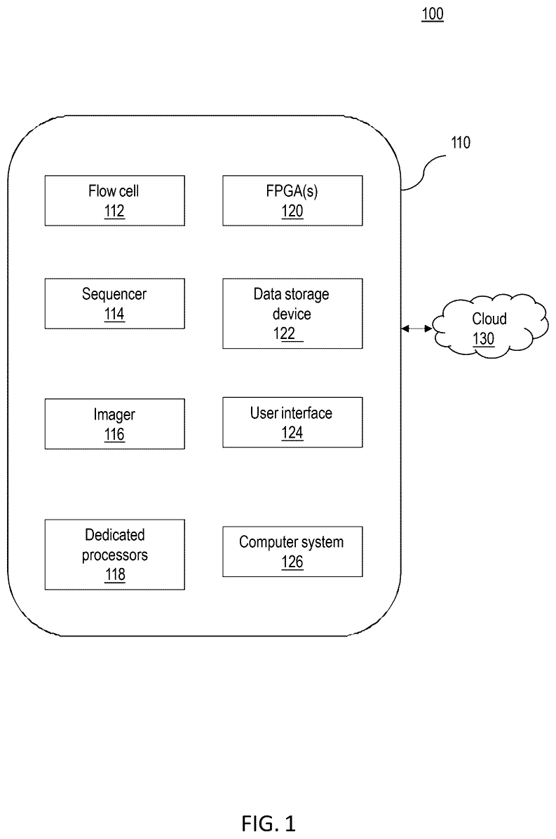

FIG. 1 illustrates a block diagram of a computer-implemented system 100, according to one or more aspects disclosed herein. The system 100 has a sequencing system 110 that includes a flow cell 112, a sequencer 114, an imager 116, data storage 122, and user interface 124. The sequencing system 110 may be connected to a cloud 130. The sequencing system 110 may include one or more of dedicated processors 118, Field-Programmable Gate Array(s) (FPGAs) 120, and a computer system 126.

In some aspects, the flow cell 112 is configured to capture DNA fragments and form DNA sequences for base-calling on the flow cell. The flow cell 112 may include the support as disclosed herein. The support may be a solid support. The support may include a surface coating thereon as disclosed herein. The surface coating may be a polymer coating as disclosed herein.

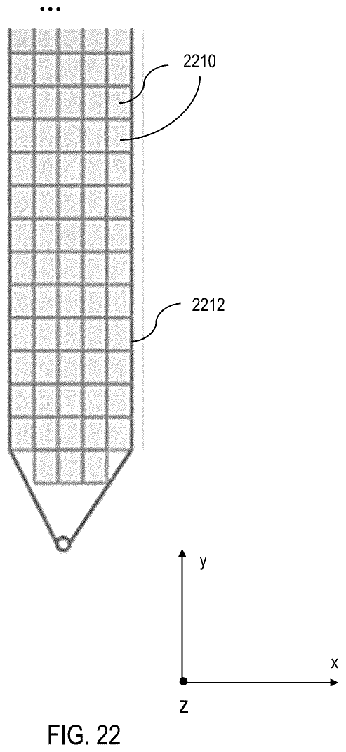

A flow cell 112 may include multiple tiles or imaging areas thereon, and each tile may be separated into a grid of subtiles. Each subtile may include a plurality of clusters or polonies thereon. As a non-limiting example, a flow cell may have 424 tiles, and each tile may be divided into a 6*9 grid, therefore 54 subtiles. The flow cell image as disclosed herein may be an image including signals of a plurality of clusters or polonies. The flow cell image may include one or more tiles of signals or one or more subtiles of signals. In some aspects, each tile or subtile may include millions of polonies or clusters. As a nonlimiting example, a tile may include about 1 to 10 million of clusters or polonies. Each polony may be a collection of many copies of DNA fragments. In some aspects, a flow cell image may be an image that includes all the tiles and approximately all signals thereon. The flow cell image may be acquired from a channel during an imaging or sequencing cycle using the imager 116. Depending on the sample(s) immobilized on the support or flow cell, the flow cell images may include multiple z levels which are orthogonal to the image plane of the flow cell images. In particular, for three dimensional (3D) samples, e.g., cells, tissues, or other in situ samples, the flow cell images can include multiple z-levels in order to cover the whole sample(s) in 3D. The z axis can extend from the objective lens of the optical system disclosed herein to the support, e.g., flow cell. The axial axis can be orthogonal to the image plane of the flow cell images. Each z level of flow cell images may be separated from the adjacent z level(s) for a predetermined distance, for example, for about 0.1 um to about 15 ums. Each z level of flow cell images may be separated from the adjacent level(s) for 1 um to 10 ums. At each z-level, a flow cell image can be acquired from one or more sequencing cycles and/or one or more channels. Each flow cell image may include in its field of view at least part of one or more tiles or subtiles of the flow cell. FIG. 22 shows a portion of a flow cell 2212 with multiple tiles 2210. The image plane is defined by the x and y axis. And the z axis is orthogonal to the x-y plane. Although the flow cell images, samples, and the axial axis are described in a Cartesian coordinate system as shown in FIG. 22, any other coordinate systems can be used to define spatial locations and relationships herein. Other coordinate systems can include but are not limited to the polar coordinate system, cylindrical, or spherical coordinate systems.

The sequencer 114 may be configured to flow a nucleotide mixture onto the flow cell 112, cleave blockers from the nucleotides in between flowing steps, and perform other steps for the formation of the DNA sequences on the flow cell 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some aspects, the sequencer 114 and the flow cell 112 may be configured to perform various sequencing methods disclosed herein, for example, sequencing-by-avidity.

For example, each nucleotide base may be assigned a color. Different types of nucleotides may have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 116 may be configured to capture images of the flow cell 112 after each flowing step. In an aspect, the imager 116 is a camera configured to capture digital images, such as an active-pixel sensor (CMOS) or a CCD camera. The camera may be configured to capture images at the wavelengths of the fluorescent elements bound to the nucleotides. The images may be called flow cell images.

In some aspects, the imager 116 may include one or more optical systems disclosed herein. The optical system(s) may be configured to capture optical signals from the flow cell and generate corresponding digital images thereof. The digital images may then be used for base calling.

In an aspect, the images of the flow cell may be captured in groups, where each image in the group is taken at a wavelength or in a spectrum that matches or includes only one of the fluorescent elements. In another aspect, the images may be captured as single images that captures all of the wavelengths of the fluorescent elements.

The resolution of the imager 116 controls the level of detail in the flow cell images, including pixel size. In existing systems, this resolution is very important, as it controls the accuracy with which a spot-finding algorithm identifies the polony centers. One way to increase the accuracy of spot finding is to improve the resolution of the imager 116 (e.g., by incorporating a higher-resolution camera), or improve the processing performed on images taken by imager 116. Detecting polony centers in pixels other than those detected by a spot-finding algorithm may be performed. These processing-based methods may allow for improved accuracy in detection of polony centers without increasing the resolution of the imager 116. The resolution of the imager may even be less than existing systems with comparable performance, which may reduce the cost of the sequencing system 110. In some aspects, the resolution of the imager may be the same as existing systems but achieve superior performance as compared to those existing systems due to the image processing.

The image quality of the flow cell images controls the base calling quality. One way to increase the accuracy of base calling is to improve the imager 116, or improve the processing performed on images taken by imager 116 to result in a better image quality. The methods described herein correct the image intensities to remove phasing and prephasing effects on the image intensities, so that the base calling based on the corrected image intensities may be more accurate than without such corrections. These methods may allow for accurate and efficient phasing and prephasing correction. Further, since the methods disclosed here are computationally less intensive than traditional methods, heat dissipation by the computer/processors may be easier to manage so that it is unlikely to cause undesired shift from the proper chemistry of sequencing techniques disclosed herein. These methods may be advantageously performed in parallel in the computer-implemented system 100, without interference with or delay of existing sequencing workflow of the system 100. The results of phasing and prephasing correction may be available for making actual base calls in the current cycle in the sequencing workflow. Further, some of the operations disclosed herein may be advantageously performed by the FPGA(s) and data may be communicated between the CPU(s) and FPGA(s) to reduce the total operational time from methods operating only using the CPU(s). Furthermore, instead of processing directly on multiple raw flow cell images, image intensities from polonies are extracted from the images and may be more efficiently handled than the images themselves for base calling.

The sequencing system 110 may be configured to perform phasing and prephasing corrections based on image intensities of polonies on the flow cell images. The operations or actions disclosed herein may be performed by the dedicated processors 118, the FPGA(s) 120, the computing system 126, or a combination thereof. One or more operations or actions in methods 500, 600, 2300 disclosed herein may be performed by the dedicated processors 118, the FPGA(s) 120, the computing system 126, or a combination thereof. In some aspects, which operations or actions are to be performed by performed by the dedicated processors 118, the FPGA(s) 120, the computing system 126, or their combinations may be determined based on one or more of: a computation time for the specific operation(s), the complexity of computation in the specific operation(s), the need for data transmission between the hardware devices, or their combinations. Phasing and prephasing correction of base calling may be performed after the flow cell images are acquired, but before actual base calling of the flow cell images is performed in a cycle.

The computing system 126 may include one or more general purpose computers that provide interfaces to run a variety of programs in an operating system, such as Windows™ or Linux™. Such an operating system typically provides great flexibility to a user.

In some aspects, the dedicated processors 118 may be configured to perform operations in the methods of phasing and prephasing corrections. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some aspects, the FPGA(s) 120 may be configured to perform operations of the phasing and prephasing correction methods herein. An FPGA is programmed as hardware that will only perform a specific task. A special programming language may be used to transform software steps into hardware componentry. Once an FPGA is programmed, the hardware directly processes digital data that is provided to it without running software. Instead, the FPGA uses logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 120 may be configured to perform the steps in parallel. For example, a number of FPGA(s) 120 may be configured to perform a processing step for an image, a set of images, or a polony location in one or more images. Each FPGA(s) 120 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems that may need to store the data before it may be processed, which may require more memory or accessing a computer system located in the cloud 130.

In some aspects, the data storage 122 is used to store information used in the phasing and prephasing correction methods. This information may include the images themselves or information derived from the images (e.g., pixel intensities, colors, etc.) captured by the imager 116. The DNA sequences determined from the base-calling may be stored in the data storage 122. Parameters identifying polony locations may also be stored in the data storage 122. Raw and/or processed image intensities of each polony may be stored in the data storage. Phasing coefficients, prephasing coefficients, and the base calls may also be stored in the data storage 122.

The user interface 124 may be used by a user to operate the sequencing system or access data stored in the data storage 122 or the computer system 126.

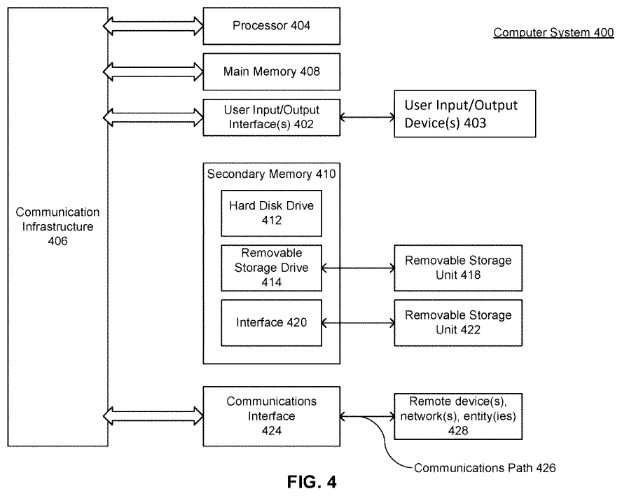

The computer system 126 may control the general operation of the sequencing system and may be coupled to the user interface 124. It may also perform steps in the phasing and prephasing correction and subsequent operations including but not limited to base-calling. In some aspects, the computer system 126 is a computer system 400, as described in more detail in FIG. 4. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish necessary processing described above. In some aspects, when these elements are present together, the processing tasks are split between them. For example, the FPGA(s) 120 may be used to perform some or all of: the preprocessing operations, the calculation of image intensities using the phasing and prephasing coefficient, and normalization of the image intensities, while the computer system 126 may perform other processing functions for the sequencing system 110. *Those skilled in the art will understand that various combinations of these elements will allow various system aspects that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

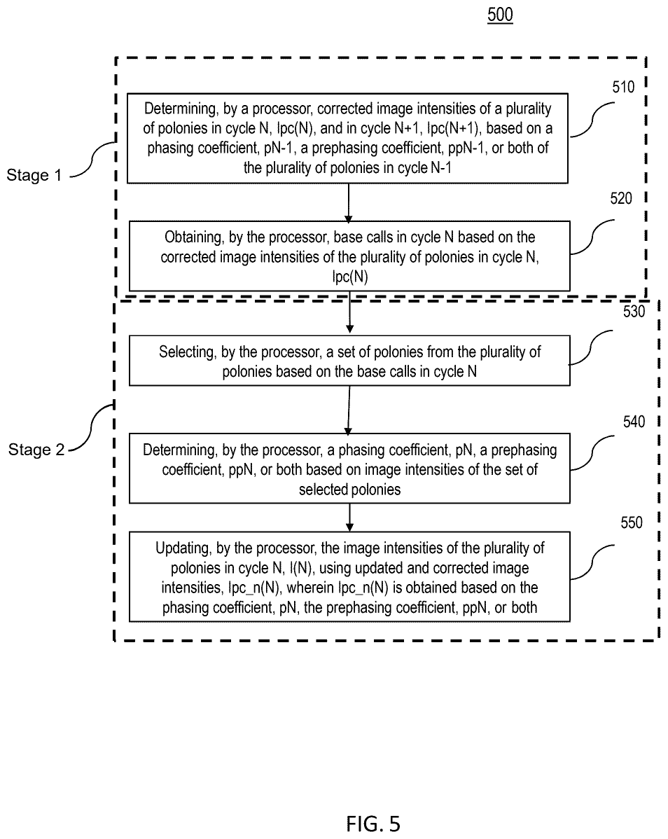

FIG. 5 illustrates a flow chart of a method for performing phasing and prephasing corrections with two stages, according to some aspects. The method 500 may include some or all of the operations disclosed herein. The operations may be performed in the order that is described herein, but is not limited to the order that has been described herein.

For each sequencing cycle, the method 500 may be a two-stage method that makes preliminary base calls in the first stage and then uses the preliminary base calls to generate phasing and prephasing coefficients and/or updating the population of polonies that may be used for making updated base calls in the second stage.

The method 500 may be performed by one or more processors disclosed herein. In some aspects, the processor may include one or more of: a processing unit, an integrated circuit, or their combinations. For example, the processing unit may include a central processing unit (CPU) and/or a graphic processing unit (GPU). The integrated circuit may include a chip such as a field-programmable gate array (FPGA). In some aspects, the processor may include the computing system 400.

In some aspects, some or all operations in method 500 may be performed by the FPGAs. In some aspects, when some operations are performed by FPGAs, the data after an operation performed by the FPGA may be communicated by the FPGAs to the CPUs so that CPUs may perform subsequent operation(s) in method 500 using such data. In some aspects, all the operations in method 500 may be performed by CPUs. Alternatively, the operations performed by CPUs may be performed by other processors such as the dedicated processors, or GPUs.

In some aspects, the method 500 is configured to correct phasing and prephasing of a plurality of polonies or clusters. The plurality of polonies or clusters may be extracted (e.g., their locations and/or corresponding intensities may be identified) from flow cell images acquired from one or more channels. For example, the plurality of polonies may be extracted from flow cell images from 4 different channels. The extraction may include a list of intensity values with one value per location per image. As another example, the plurality of polonies may be extracted from flow cell images from a single channel. An example technique for extracting the plurality of polonies in a flow cell is described in U.S. Pat. No. 11,200,446, which is hereby incorporated by reference in its entirety. The flow cell image as disclosed herein can be an image that is acquired using a support, e.g., a flow cell 112 as shown in FIG. 1. In some embodiments, the plurality of polonies or clusters may be extracted from specific regions of a tile, e.g., each subtile. With each subtile, the polonies may be extracted with a predetermined pattern or randomly.

The method 500, 600, 2300 may allow correction of phasing and prephasing of a plurality of polonies or clusters even if the polonies or clusters are of low or unbalanced diversity in sequencing cycle(s). The nucleotide diversity of a population of immobilized polonies or clusters can refer to the relative proportion of nucleotides A, G, C and T/U that are present in each sequencing cycle. An optimal high diversity library can generally include approximately equal proportions of all four types of nucleotides represented in each cycle of a sequencing run. A low diversity library can generally include a high proportion of certain nucleotide types and low proportion of other nucleotide types in one or more sequencing cycles.

In some embodiments, the polonies or clusters being sequenced in a flow cycle may have a certain nucleotide diversity. The nucleotide diversity of a population of nucleotide acid molecules, e.g., polonies or clusters, can refer to the relative proportion of nucleotides A, G, C, and T/U that are present in each flow cycle. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have a greater number of brighter spots (polonies) than images corresponding to the low portion of certain nucleotides. As an example of low or unbalanced diversity data in a flow cycle, the bases A, T, C, G can be about 1%, about 2%, about 1%, and about 95%, respectively, of the total number of polonies, in a certain flow cycle. As another example of low or unbalanced diversity data, the bases A, T, C, G in polonies at multiple flow cycles can be about 2%, about 5%, about 10%, and about 83%, respectively. In embodiments where low or unbalanced diversity data is present in a sequencing run and is imaged for sequencing analysis, image registration failure may occur because image(s) from one or more channels are too dark (e.g., signal spots of polonies are too sparse and/or dim) comparing with images acquired from other channels.

In addition to the base biases affecting diversity, plexity can also be a factor that when plexity is lower than a number, e.g., 8 or 16, the signal could be of low diversity. For example, in a 2-cycle sequence, all polonies are of AT or TG or GC or CA. It is 25% for every base in every cycle, but its plexity is less than 8, and the sequence is not all random. In some embodiments, the method 500 is configured to register flow cell images even if the polonies are of low diversity or low plexity.

In general, plexity can indicate source(s) of the sample. A uniplex sample may include DNA fragments or molecules from a same sample region in a genome or a same sample source. A multiplex sample may include DNA fragments or molecules from different sample sources, e.g., liver, kidney, heart, cancerous tissue, etc, or from one or more sample regions in the genome.

In some aspects, the method 500 is performed during a cycle N, so that base calling of cycles prior to cycle N (e.g., cycle N−1) has already been performed, while base calling of cycle N (and similarly, cycle N+1, N+2) is yet to be performed. In some aspects, cycle N is the current cycle. While sequencing of the current cycle N is being performed, the base calls of cycle(s) prior to cycle N may have been saved to a memory or a data storage device disclosed herein. The base calls of cycle(s) prior to cycle N may be loaded from the memory or data storage device. N may be any integer that is greater than 2. For example, for short read sequencing, N may be any integer from 2 to 150 or 2 to 300. In some aspects, the method 500, 600, 2300 may allow correction of phasing and prephasing of flow cell images of in situ sample(s). In situ sample(s) may include the cellular sample disclosed herein which has a depth along an axial direction (i.e., the z axis in FIG. 22) orthogonal to the image plane of flow cell images. The in situ sample(s) may have a 3D volume and the polonies or clusters may be distributed in the 3D volume therein. To image optical signals from polonies or clusters, flow cell images may be acquired at multiple axial locations spaced part from each other along the axial direction. In some aspects, a 3D polony map may be used to identify the polonies or clusters that need phasing and/or prephasing corrections. The 3D polony map may be generated in one or more flow cycles, e.g., in any one or more of the first 1 to 10 cycles, which can include all the polonies or clusters in the 3D sample being sequenced and can exclude duplicate polonies or clusters that are out of focus in the flow cell images. The duplicate polonies or clusters may cause errors in phasing and/or prephasing if they are not removed. Further, the 3D polony map may indicate the 3D position of polonies or clusters in in situ samples for accurate phasing and/or prephasing corrections. For example, polony i may appear in two flow cell images that are acquired in two adjacent axial locations. Polony i may be out of focus at z1 location and in focus at z2 location. The 3D polony map may only indicate a single polony at z2 location, so that the image intensities at the z2 location with spatial coordinates (xi, yi, z2) can be used for phasing and/or prephasing corrections. The 3D polony map may be determined as described in, for example, U.S. Patent Application No. 63/413,864, which is incorporated by reference herein in its entirety.

In some aspects, the methods 500, 600, 2300 enable phasing and prephasing correction of image intensities multiple polonies or clusters, e.g., polonies within a tile or a portion of a tile, using the same values of phase and/or prephasing coefficients, pN and ppN. It is advantageous computationally and reduces time delay in performing correction than estimating phasing and/or prephasing corrections individually for each polony or cluster.

In some aspects, the method 500 may include an operation 510 of determining corrected image intensities of a plurality of polonies in cycle N, Ipc(N), and in a cycle N+1, Ipc(N+1). The Ipc(N) may be determined based on a phasing coefficient corresponding to cycle N−1, pN−1, a prephasing coefficient corresponding to cycle N−1, ppN−1, or both the phasing coefficient and the prephasing coefficient of the plurality of polonies from cycle N−1. Cycle N−1 is immediately prior to the current cycle N.

In some aspects, the method 500 may include an operation of determining the phasing coefficient, pN−1, and the prephasing coefficient, ppN−1, of the plurality of polonies in cycle N−1. N can be any integer that is greater than 2. For example, for short read sequencing, N can be any integer from 2 to 150.

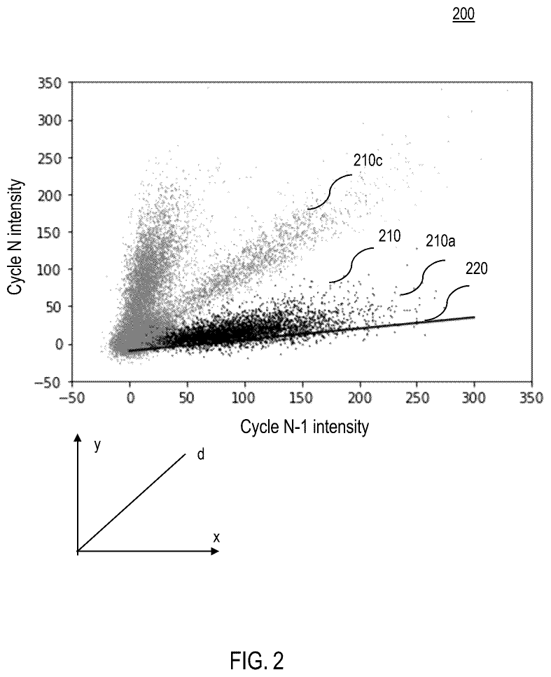

The phasing coefficient, pN−1, and prephasing coefficient, ppN−1 may be determined using image intensities of polonies in cycle N−1. For example, the coefficient ppN−1 may be determined based on the slope of a fitted function 221 as shown in FIG. 2 of image intensities in cycle N−1, I(N−1), which indicate multiple polonies are that are supposed to be “on” in cycle N but prephased into cycle N−1 and are partially or completely “on” in cycle N−1, as a result of different copies of DNA fragments are either “on” in cycle N−1 or in cycle N. Alternatively, the coefficients, pN−1 and ppN−1, may be determined using method 500 similarly as how pN and ppN is determined as disclosed below by setting the current cycle as N−1. If image intensity of N−2 is not available, e.g., N=2, then I(N−2) is set as zero. In general, when N is small, e.g., N=2, 3, or 4, etc., the phasing coefficient and prephasing coefficient may be assumed to be approximately 0.

Before the operation 510, the methods 500 may comprise an operation of providing a plurality of nucleic acid template molecules immobilized on a support. Each nucleic acid template molecule may comprise an insert sequence of interest. The insert sequence can be different in different template molecules. Each template molecule may correspond to a polony of optical signals in flow cell images.

Before the operation of 510, the methods 500 may comprise an operation of generating flow cell images by conducting one or more cycles of sequencing reactions of the plurality of nucleic acid template molecules immobilized on the support. The flow cell images can be generated or acquired by the sequencing system disclosed herein. Conducting the one or more cycles of the sequencing reactions may comprise contacting the plurality of nucleotide acid template molecules using a plurality of nucleotide reagents comprising a mixture of different types of nucleotide bases A, G, C and T/U. Individual nucleotide reagent may comprise a different detectable color label that corresponds with each different type of nucleotide base.

In some embodiments, conducting the one or more cycles of the sequencing reactions may comprise: contacting the plurality of nucleotide acid template molecules with a plurality of sequencing primers, a plurality of polymerases and a mixture of different types of avidites. An individual avidite in the mixture may comprise a core attached with multiple nucleotide arms and each arm of the individual avidite comprises the same type of nucleotide base. In some embodiments, conducting the one or more cycles of the sequencing reactions comprises: in each of the one or more cycles, imaging optical color signals emitted from nucleotide reagents that are bound to the plurality of template molecules. Imaging the optical signals may be performed by an optical system, e.g., the imager 116, disclosed herein. In some embodiments conducting the one or more cycles of the sequencing reactions may comprise: in each of the one or more cycles, acquiring the flow cell images comprising optical color signals emitted from nucleotide reagents that are bound to the plurality of template molecules.

In some embodiments, the flow cell images comprises optical signals emitted from nucleotide reagents bound to a unbalanced diversity of nucleotide bases of A, G, C and T/U among the plurality of nucleic acid template molecules immobilized on the support in the one or more cycles. In some embodiments, the plurality of polonies comprise a unbalanced diversity of nucleotide bases of A, G, C and T/U, and wherein the unbalanced diversity comprises a percentage of: (1) a number of one or more types of nucleotide bases to (2) a total number of nucleotide bases, and the percentage is less than 20%, 15%, 10%, or 5% in the cycle N.

Before the operation of 510, the method 500 may include an operation of obtaining image intensities of the plurality of polonies in the cycles N−1, N, N+1, N+2, N−2, or their combinations. The image intensities may be determined as described in, for example, U.S. Pat. No. 11,200,446, which is incorporated by reference herein in its entirety.

FIG. 2 is a scatter plot 200 illustrating image intensity of polonies in cycles N, I(N), and in cycle N−1, I(N−1) of a single color channel. Each dot represents a polony 210. The polony 210 corresponds to an image intensity in cycle N and an image intensity in cycle N−1. The image intensity may be determined after one or more preprocessing steps disclosed herein. The image intensity may be normalized and/or scaled to be within a specific range, e.g., [0, 400]. The dots gathered near the origin of (0, 0) represent polonies whose image intensities are dark or approximately zero in both cycles N and N−1. Such polonies are “off” in both cycles N and N−1, i.e., the base of these polonies does not correspond to the channel in both cycles.

Ideally, without phasing or prephasing contamination, a polony that has a base that corresponds to the color channel is “on” in cycle N and has a non-zero image intensity in cycle N. The same polony should not contain a signal that is phased into cycle N from cycle N−1. In other words, the actual signal in cycle N should exclude the signal that should be “on” in cycle N−1 but delayed at least partly into cycle N due to phasing. *Such population of polonies 210a are black dots at or near the bottom in FIG. 2 and close to the horizontal axis, x′. Similarly, the actual signal of cycle N−1 should exclude the signal that should be “on” in cycle N−1 but advanced at least partly into cycle N due to prephasing. A relatively small population of polonies 210c are “on” for both cycles and are distributed approximately along a diagonal axis, d. Phasing and/or prephasing may also have an effect on image intensities polonies around the diagonal axis, d, but not calculated since two bases in cycle N and cycle N−1 are identical.

A polony may have many copies of the DNA fragments/molecules used for sequencing, but phasing may cause some copies to go out of synchronization, thereby shifting a polony from being “on” in cycle N−1 to be “on” in cycle N and “off” in cycle N−1. Depending on how many copies of DNA fragments/molecules are shifted in a total number of copies in an individual polony, a polony may be shifted from 100% “on” in cycle N−1 to be partially “on” (e.g., 20% “on”) in cycle N−1 or 100% “off” in cycle N−1. This can cause phasing contamination of the image intensity in cycle N. Similarly, a cluster with multiple repetitions or molecules containing the same DNA fragments may have phasing contamination of the image intensity in cycle N. As a result, the image intensities of polonies or clusters in cycle N shift away from their ideal locations around the horizontal axis, x′. A linear function 220 may be fit to a selected population of polonies, and the slope of the linear function may correspond to the level of phasing of the polonies in cycle N. The image intensity caused by such phasing contaminations may be removed using the phasing coefficient, pN, and the image intensity in cycle N−1, I(N−1).

Similarly, prephasing may cause some copies of DNA molecules in an individual polony to go out of synchronization thereby shifting from being “on” in cycle N+1 to be “on” in cycle N instead, so that the corresponding polony may be partially or completely “on” in cycle N and/or cycle N+1. The image intensity caused by such prephasing contaminations may be removed using the prephasing coefficient, ppN and the image intensity in cycle N+1, I(N+1).

In some aspects, this operation of performing one or more preprocessing steps may be performed by the FPGAs. In some aspects, the data after the operation may be communicated by the FPGAs to the CPUs so that CPUs may perform subsequent operation(s) in method 500 using such data. In some aspects, the one or more preprocessing steps may be performed before operation 510. In some aspects, the one or more preprocessing steps may after operation 510 but before operation 530.

The one or more preprocessing steps may comprise background subtraction. The background subtraction can be configured to remove at least some background signal that may interfere with the signal of interest, i.e., image intensities of the polonies. The background signal may be noise caused by multiple sources including the flow cell 112, the imager 115, the sequencer 114, and/or other sources. The background subtraction may be adjusted to avoid over subtraction.

The one or more preprocessing steps may include image sharpening so that image intensities of polonies may be optimized in consideration of their surroundings in the flow cell images. For example, a Laplacian of Gaussian (LoG) filter may be used for sharpening.

The one or more preprocessing steps may include image registration so that image intensities of polonies may be registered relative to each other. For example, the image intensities may be registered to the template as disclosed herein. An example technique for generating the template is described in U.S. Pat. No. 11,200,446, which is hereby incorporated by reference in its entirety. The template can be in two dimensions (2D). In embodiments where flow cell images are acquired from 3D samples at multiple z levels, the template can be in 3D.

The one or more preprocessing steps may include intensity extraction in which, polonies with their corresponding intensities are extracted from the 2D image into a different data format that is simpler and more efficient to handle. For example, each polony may have 4 different intensities, each intensity from a different channel. Such intensities may be extracted into a one-dimensional (1D) list, with each entry of the list corresponding to a polony so that the spatial relationship between polonies, e.g., neighboring polonies, is eliminated. The list may be generated after image registration to reflect location information of the same polonies in different cycles. As such, image intensities of the same polony in different cycles may be located in different lists each corresponding to a cycle.

The one or more preprocessing steps may include intensity offset adjustment that may remove the offset in the intensity that has not been removed during background subtraction.

The one or more preprocessing steps may include color correction to remove interference or cross-talk of one color channel from other channels or colors.

In some aspects, the operation of performing the one or more preprocessing steps occurs before determining the phasing coefficient, pN−1, the prephasing coefficient, ppN−1, or both. In other aspects, performing the one or more preprocessing steps occurs before selecting the set of polonies from the plurality of polonies.

Returning to operation 510, in some aspects, the corrected image intensities for cycle N, Ipc(N), may be obtained as:

$${I}_{p c} ( N ) = I ( N ) - {p}_{N - 1} * I ( N - 1 ) - {pp}_{N - 1} * I ( N + 1 ) \qquad ( 1 )$$

where pN−1 is the phasing coefficient of cycle N−1, ppN−1 is the prephasing coefficient of cycle N−1, I(N) is the image intensity of the polony in cycle N, and I(N+1) is the intensity of the same polony in cycle N+1. Ideally, when there is no prephasing or phasing, the copies of DNA fragments in the polony are synchronized, and the image intensity I(N) may be reliably used for base calling, e.g., determining what base it is in cycle N. When there is phasing or prephasing, there may a portion of copies or otherwise repetitions of DNA fragments/molecules that should be “on” in cycle N but instead is “off” because of the asynchrony. Signals from such copies may be removed by using equation (1) to correct for the phasing and/or prephasing effect in the image intensity, I(N). In some embodiments, equation (1) can be simplified by setting the image intensities in cycle N−1, I(N−1), or in cycle (N+1), I(N+1) to zero, so that only phasing or prephasing, but not both are corrected. For example, the method 500 may include an operation to determine whether phasing, prephasing, or both corrections would be needed, and in response to a determination that prephasing is below a predetermined threshold, I(N+1) can be set to zero, and no determination of ppN−1 or ppN is needed.

Similarly, in some aspects, the corrected image intensities in cycle N+1, Ipc(N+1), for each polony may be obtained as:

$${I}_{p c} ( N + 1 ) = I ( N + 1 ) - {p}_{N - 1} * I ( N ) - {pp}_{N - 1} * I ( N + 2 ) \qquad ( 2 )$$

where pN−1 is the phasing coefficient of cycle N−1, ppN−1 is the prephasing coefficient of cycle N−1, I(N), is the image intensity of the polony in cycle N, I(N+1), is the intensity of the same polony in cycle N+1, I(N+2), is the intensity of the same polony in cycle N+2. In some aspects, I(N+2) may be set as zero to simplify the estimation in equation (2) or when I(N+2) is unavailable. Equation (2) is used when the phasing coefficient of cycle N, pN, and the prephasing coefficient of cycle N, ppN, are not available. If pN and ppN, or one of them is available, the corrected image intensities in cycle N, Ipc(N+1), for each polony may be obtained as:

$${I}_{p c} ( N + 1 ) = I ( N + 1 ) - {p}_{N} * I ( N ) - p {p}_{N} * I ( N + 2 ) \qquad ( 3 )$$

where pN is the phasing coefficient of cycle N, ppN is the prephasing coefficient of cycle N, I(N), is the image intensity of the polony in cycle N, I(N+1), is the intensity of the same polony in cycle N+1, I(N+2), is the intensity of the same polony in cycle N+2. In some aspects, I(N+2) may be set as zero, to simplify the estimation in equation (3) (ppN does not need to be determined if I(N+2) is zero or unavailable).

The method 500 may further comprise (e.g., as part of operation 510) an operation of generating normalized image intensities, Inorm(N), of cycle N by normalizing the corrected image intensities in cycle N, Ipc(N). In some aspects, Inorm(N)=F(Ipc(N), f(N)), wherein FO is a function, and f(N) is the normalization factor of cycle N. Normalizing Ipc(N) may include dividing Ipc(N) by a normalization factor. The normalization factor may be predetermined. For example, the normalization factor may be the same as the normalization factor in the previous cycle, cycle N−1. In some aspects, the normalization factor may be determined based on the image intensities Ipc(N) of some or all of the plurality of polonies. As an example, the predetermined normalization factor is an image intensity at about 99^{th} percentile of the image intensity of the brightest polony among all of the plurality of polonies in cycle N. As another example, the predetermined normalization factor can be in a range of about 30^{th} percentile to about 99^{th} percentile of the brightest image intensity of polonies in cycle N.

Similarly, the method 500 may further comprise (e.g., as part of operation 510) an operation of generating normalized image intensities, Inorm(N+1), of cycle N+1 by normalizing Ipc(N+1) by a normalization factor, f(N+1). In some aspects, Inorm(N+1)=F(Ipc(N+1), f(N+1)), wherein F( ) is a function, and f(N+1) is the normalization factor of cycle N+1. The normalization factor may be predetermined. For example, the normalization factor may be the same as the normalization factor in a previous cycle, e.g., cycle N−1. The normalization factor may be determined based on the image intensities I_{pc}(N+1) of some or all of the plurality of polonies. As an example, the predetermined normalization factor is an image intensity at about 99^{th} percentile of the image intensity of the brightest polony among all of the plurality of polonies in cycle N+1. The predetermined normalization factor can be in a range of about 30^{th} percentile to about 99^{th} percentile of the brightest image intensity of polonies in cycleN+1.

After the corrected image intensities have been determined in operation 510, the method 500 may comprise an operation 520 of making the base calls in cycle N using the corrected image intensities of the plurality of polonies in cycle N, Ipc(N), and making the base calls in cycle N+1 using the corrected image intensities of the plurality of polonies in cycle N+1, Ipc(N+1) using various base calling algorithms. In some aspects, the base calls of cycle N and N+1 from Ipc(N) and Ipc(N+1) are preliminary base calls in a first stage of the two-stage method 500.

In some aspects, the method 500 may comprise (e.g., as part of operation 520) an operation of making the base calls in cycle N using the normalized image intensities, Inorm(N), and making the base calls in cycle N+1 using the normalized image intensities, Inorm(N+1). After normalization, the image intensities across different channels may be more comparable in range than those before normalization. Such normalization before base calling may help to make more accurate and reliable base calling from different channels. In some aspects, the base calls of cycle N and N+1 from the normalized image intensity, Inorm(N) and Inorm (N+1), are preliminary base calls in a first stage of the two-stage method 500.

The method 500 may comprise (e.g., as part of operation 520) an operation of loading, receiving, or making the base calls in cycle N−1. For example, the base calls may be made by a CPU and communicated to the processor performing some or all of the operations of method 500. Alternatively, the processor may make base calls based on the image intensity of cycle N−1, I(N−1). The base calls of cycle N−1 may be made from image intensity, I Ipc(N−1) or Inorm(N−1). The base calls of cycle N−1 may be made using various base calling algorithms, and is not limited to a particular algorithm.

The method 500 may further include an operation 530 of selecting a set of polonies from the plurality of polonies based on: the base calls in cycle N; the base calls in cycle N+1; the base calls of cycle N−1; or a combination thereof.

With the base calls from cycles N−1 and N, a first population of polonies that corresponds to the prephasing coefficient of cycle N, ppN, may be selected. The first population of polonies may include some or approximately all the polonies that are called a base corresponding to a specific channel in cycle N−1 (“on” in cycle N−1) and not called the same base in cycle N (“off” in cycle N). The first population of polonies may be used for calculating pN, for example, by fitting the intensities to a linear function and obtaining the slope between the fitted linear function with the horizontal axis, x′, as ppN.

With the base calls from cycles N and N+1, a second population of polonies that is associated with the phasing coefficient of cycle N, pN, may be selected. The second population of polonies includes some or approximately all the polonies that are called a base corresponding to a specific channel in cycle N+1 and not called the base in cycle N. The second population of polonies may be used for calculating pN, for example, by fitting the intensities to a linear function and obtaining the slope between the fitted linear function and the vertical axis, y′, as pN.

Fitting of the population of polonies or clusters to a linear function may be achieved using various linear fitting methods such as linear regression or binned percentile method. In some aspects, fitting of the population may be selected to reflect the lower profile, e.g., fitted linear function 220 as shown in FIG. 2, in order minimize the interference between channels. Various fitting methods other than linear fitting, e.g., non-linear regression, may also be used to fit the population of polonies.

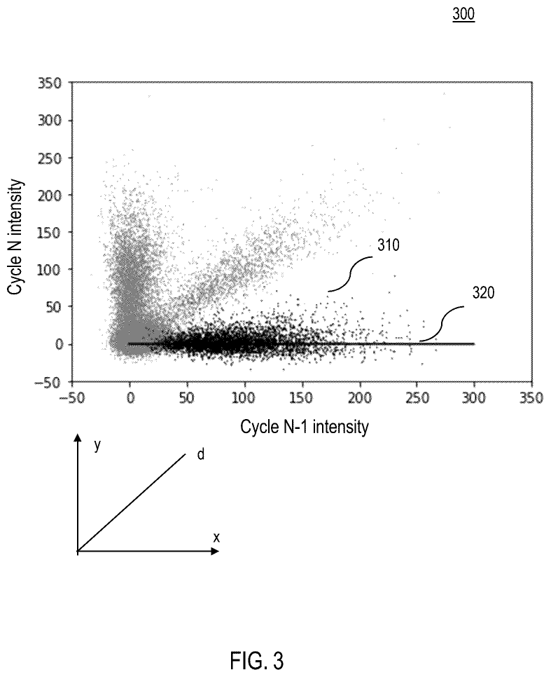

FIG. 3 is a scatter plot illustrating image intensities of polonies in cycles N and N−1, in FIG. 2, after phasing and prephasing correction using the technologies herein, according to some aspects. The corrected image intensities are Ipc(N). Image intensity of polonies are shifted back to their ideal locations around the horizontal or vertical axis with the correction using methods herein. The phasing and prephasing correction may be based on coefficients, e.g., pN and ppN, derived from the slope of the fitted linear function.

In some aspects, the phasing or prephasing coefficient, pN or ppN is a percentage in a range from 0 to about 0.9%. In some aspects, the phasing or prephasing coefficient, pN or ppN is a percentage in a range from 0 to about 0.2%. In some aspects, the phasing or prephasing coefficient, pN or ppN is a percentage in a range from 0 to about 0.25%. In some aspects, the phasing or prephasing coefficient, pN or ppN is a percentage in a range from 0 to about 0.3%. In some aspects, the phasing or prephasing coefficient, pN or ppN is a percentage in a range from 0 to about 0.35%. In some aspects, the phasing or prephasing coefficient, pN or ppN is a percentage in a range from 0 to about 0.5%. In some aspects, the phasing or prephasing coefficient, pN or ppN is a percentage in a range from 0 to about 99%. In some aspects, the phasing or prephasing coefficient, pN or ppN is a percentage in a range from 0 to about 50%.

In some aspects, the phasing or prephasing coefficient, pN or ppN is a percentage in a range from 0 to 0.9%. In some aspects, the phasing or prephasing coefficient, pN or ppN is a percentage in a range from 0 to 0.2%. In some aspects, the phasing or prephasing coefficient, pN or ppN is a percentage in a range from 0 to 0.25%. In some aspects, the phasing or prephasing coefficient, pN or ppN is a percentage in a range from 0 to 0.3%. *In some aspects, the phasing or prephasing coefficient, pN or ppN is a percentage in a range from 0 to 0.35%. In some aspects, the phasing or prephasing coefficient, pN or ppN is a percentage in a range from 0 to 0.5%. In some aspects, the phasing or prephasing coefficient, pN or ppN is a percentage in a range from 0 to 99%. In some aspects, the phasing or prephasing coefficient, pN or ppN is a percentage in a range from 0 to 50%.

In some aspects, the phasing or prephasing coefficient, pN or ppN is a coefficient measured at individual cycles, e.g., cycle N, considering phasing and prephasing from its adjacent cycles, e.g., cycles N−1, N+1. In some embodiments, the phasing or prephasing coefficient per cycle can be used cumulatively to estimate phasing and/or prephasing from cycle N to cycle M, e.g., from the first cycle to the 100^{th} cycle. For example, using first order approximation, if phasing coefficient is about 0.2% in each cycle, the 100^{th} cycle may have 20% phasing from the first cycle.

In some aspects, the method 500 may include an operation 540 of determining the phasing coefficient for the cycle N, pN, the prephasing coefficient for the cycle N, ppN, or both for each channel. Such operation 540 may include: in response to determining that pN, ppN, or both is outside of a pre-determined corresponding range, set a corresponding value for pN, ppN, or both based on a corresponding value of pN, ppN, or both from a different channel in cycle N.

In operation 550 of method 500, there may be an operation of updating the image intensities of the plurality of polonies in cycle N, I(N), using updated and corrected image intensities, Ipc_n(N).

The updated and corrected image intensity, Ipc_n(N), may be obtained as:

$${I}_{pc \_} ( N ) = I ( N ) - {p}_{N} * I ( N - 1 ) - p {p}_{N} * I ( N + 1 ) \qquad ( 4 )$$

wherein pN is the phasing coefficient of cycle N, ppN is the prephasing coefficient of cycle N, I(N) is the image intensity of the polony in cycle N, I(N+1) is the intensity of the same polony in cycle N+1, and I(N−1) is the intensity of the same polony in cycle N−1.

In some aspects, this operation 550 may be performed by the FPGAs. In some aspects, such operation may be performed by CPUs. Alternatively, such operation may be performed by one or more FPGA(s), and the corrected normalized image intensities, Ipc_n(N) may be communicated from the FPGA(s) to the CPU(s) for subsequent operations.

In some aspects, the method 500 may further comprise an operation of calculating a normalization factor. The normalization factor may be obtained from image intensities of some or all of the “on” polonies in cycle N. In some aspects, the normalization factor does not depend on the image intensities of any of the “off” polonies in cycle N. The “on” and “off” polonies may be determined based on the base calls in cycle N made in operation 520 of method 500, which are also considered as the preliminary base calls in cycle N. In some embodiments, an “on/off” threshold may be used so that polonies that satisfy the thresholds are “off.” The “on” polonies are a subset of the plurality of polonies. The “on” polonies are the polonies with base calls that match the corresponding channel. For example, a polony with a base call of “T” is “on” in the corresponding channel for “T,” but is “off” in all other channels for bases A, C, and G. The base calls for determining “on” and/or “off” polonies can be preliminary base calls. The base calls for determining “on” and/or “off” polonies can be base calls from the current cycle. In some embodiments, the “on” polonies can be polonies that are above a threshold image intensity, and the “off” polonies can be polonies that are below the same threshold or a different threshold image intensity. The threshold(s) can be customized depending on imaging parameters or various sequencing applications. For example, the threshold for “on” polonies is about 10^{th} percentile of the image intensity of the brightest polony in cycle N. As another example, the threshold for “off” polonies is about₄th percentile of the image intensity of the brightest polony in cycle N.

In some aspects, the “on” polonies are different from the first population and the second population determined in operation 530. In some aspects, the “on” polonies substantially exclude the first population and the second population determined in operation 530.

In some aspects, the method 500 may further comprise an operation of determining a normalization factor, f_on(N), using the image intensities of “on” polonies, which are called the base corresponding to the channel, but not other bases. The normalization factor, f_on(N), may be determined based on the image intensities of some or all of the “on” polonies. As an example, the predetermined normalization factor, f_on(N), is an image intensity at about 99^{th} percentile of the image intensity of the brightest polony among all of the “on” polonies in cycle N. The normalization factor may be in a range of about 30^{th} percentile to about 99^{th} percentile of the brightest image intensity of polonies in cycle N. The normalization factor may be about 60^{th} percentile of the brightest image intensity of polonies in cycle N. The normalization factor for different channels may be different. The normalization factor for each channel may be determined using all of the “on” polonies in connection with image intensities of polonies in other channels.

In some aspects, the method 500 may further comprise an operation of generating corrected normalized image intensities, Inorm_n(N) by dividing the image intensities of the “on” populations by the normalization factor.

In some aspects, the operation of generating corrected normalized image intensities, Inorm_n(N) may include: for each channel, in response to determining that Inorm_n(N) falls outside of a pre-determined corresponding range, set a value for Inorm_n(N) based on a corresponding value of Inorm_n(N) in a different channel in cycle N and an image intensity ratio between the channel and the different channel. In some aspects, the image intensity ratio may be determined in cycle N−1 or other cycles preceding cycle N.

In some aspects, the method 500 may further comprise an operation of updating the normalized image intensity, Inorm(N), by using the corrected normalized image intensities, Inorm_n(N).

In some aspects, the method 500 may further comprise an operation of updating the base calls in cycle N based on the corrected normalized image intensities, Inorm_n(N). The corrected normalized image intensities, Inorm_n(N), may be obtained as disclosed herein for each individual channel. After normalizing with the normalization factor, Inorm_n(N) for each channel may be within a same range, e.g., a range of [0, 3], so that a base calling algorithm may include comparison of image intensities of the same polony from different channels to make the base call. As an example, the channel with the highest Inorm_n(N) may be the channel that corresponds to the base in the polony. In some aspects, the corrected normalized image intensities, Inorm_n(N), may be further scaled by a scaling factor to a predetermined range before any base calling. The scaling factor may be an integer that is greater than 0. For example, the scaling factor may be 1000, 2000, 3000, 5000, or any other integer number.

In some aspects, the method 500 may further comprise an operation of updating the base calls in cycle N based on the updated and corrected image intensities, Ipc_n(N).

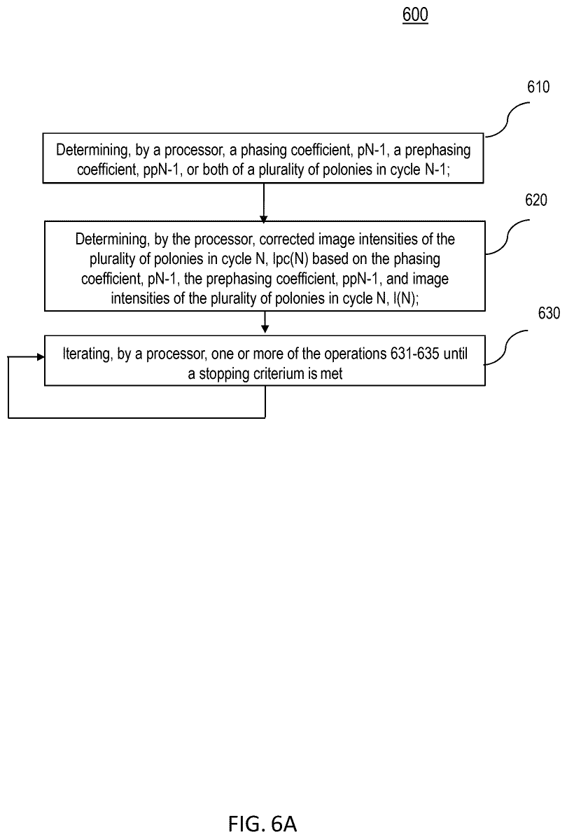

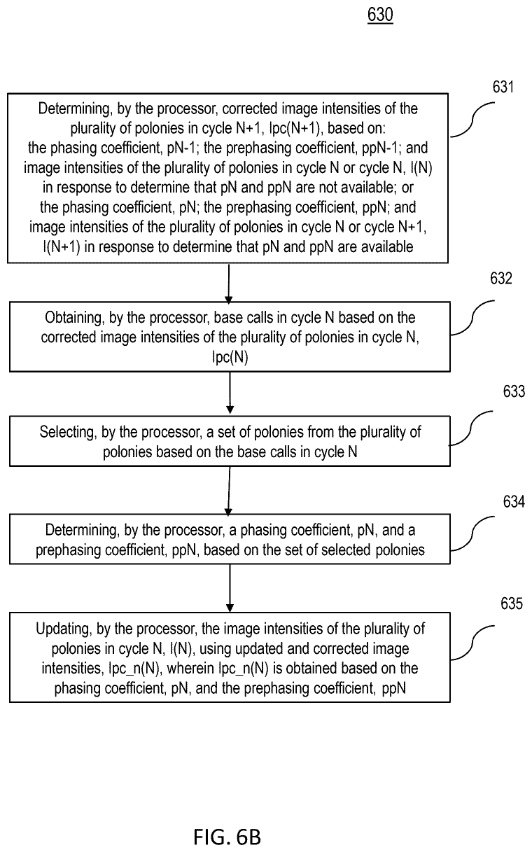

FIGS. 6A-6B show flow charts of a method 600 for performing phasing and prephasing correction with iteration(s), according to some aspects. The method 600 may include some or all of the operations disclosed herein. The operations may be performed in the order that is described herein, but is not limited to the order that has been described herein.

For each sequencing cycle, the method 600 may be a multi-stage method that makes preliminary base calls in a first stage and then uses the preliminary base calls to generate phasing and prephasing coefficients and/or updating the “on” population of polonies that may be used for making updated base calls in a second stage. The second stage may be iterated until a stopping criterion is met. A stopping criterion may be pre-determined. For example, it may be set to stop at a certain iteration, e.g., 3 to 5 iterations. Generally, a couple of iterations, e.g., 1-9, can be sufficient to ensure reliable and accurate correction. As another example, the stopping criterion may be that the difference between phasing or prephasing coefficients from two consecutive iterations is approximately zero.

The method 600 may be performed by one or more processors disclosed herein. In some aspects, the processor may include one or more of: a processing unit, an integrated circuit, or their combinations. For example, the processing unit may include a central processing unit (CPU) and/or a graphic processing unit (GPU). The integrated circuit may include a chip such as a field-programmable gate array (FPGA). In some aspects, the processor may include the computing system 400.

In some aspects, some or all operations in method 600 may be performed by the FPGAs. In some aspects when some operations are performed by FPGAs, the data after an operation performed by the FPGA may be communicated by the FPGAs to the CPUs so that CPUs may perform subsequent operation(s) in method 500 using such data. In some aspects, all the operations in method 500 may be performed by CPUs. Alternatively, the operations performed by CPUs may be performed by other processors such as the dedicated processors, or GPUs.

In some aspects, the method 600 is configured to correct phasing and prephasing of a plurality of polonies. The plurality of polonies may be extracted from flow cell images from one or more channels. For example, the plurality of polonies may be extracted from flow cell images from 4 different channels. As another example, the plurality of polonies may be extracted from flow cell images from a single channel. The flow cell image as disclosed herein is an image that is acquired using a flow cell 112 as shown in FIG. 1.

In some embodiments, the polonies or clusters being sequenced in a flow cycle may have a certain nucleotide diversity. The nucleotide diversity of a population of nucleotide acid molecules, e.g., polonies or clusters, can refer to the relative proportion of nucleotides A, G, C, and T/U that are present in each flow cycle. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have a greater number of brighter spots (polonies) than images corresponding to the low portion of certain nucleotides. As an example of low or unbalanced diversity data in a flow cycle, the bases A, T, C, G can be about 1%, about 2%, about 1%, and about 95%, respectively, of the total number of polonies, in a certain flow cycle. As another example of low or unbalanced diversity data, the bases A, T, C, G in polonies at multiple flow cycles can be about 2%, about 5%, about 10%, and about 83%, respectively. In embodiments where low or unbalanced diversity data is present in a sequencing run and is imaged for sequencing analysis, image registration failure may occur because image(s) from one or more channels are too dark (e.g., signal spots of polonies are too sparse and/or dim) comparing with images acquired from other channels.

In addition to the base biases affecting diversity, plexity can also be a factor that when plexity is lower than a number, e.g., 8 or 16, the signal could be of low diversity. For example, in a 2-cycle sequence, all polonies are of AT or TG or GC or CA. It is 25% for every base in every cycle, but its plexity is less than 8, and the sequence is not all random. In some embodiments, the methods 500 is configured to register flow cell images even if the polonies are of low diversity or low plexity.

In general, plexity can indicate source(s) of the sample. A uniplex sample may include DNA fragments or molecules from a same sample region in a genome or a same sample source. A multiplex sample may include DNA fragments or molecules from different sample sources, e.g., liver, kidney, heart, cancerous tissue, etc., or from one or more sample regions in the genome.

The method 500, 600, 2300 is configured to correct phasing and prephasing of a plurality of polonies or clusters even if the polonies are low diversity data.

In some aspects, the method 600 is performed during cycle N, so that base calling of cycles prior to cycle N has been performed, while base calling of cycle N is yet to be performed. In some aspects, cycle N is the current cycle. In some aspects, the iteration of method 600 is for the current cycle N.

In some aspects, the method 600 may include an operation 610, which is similar to the operation of determining corrected image intensities, Ipc(N), disclosed in method 500.

Before the operation 610, the method 600 may comprise an operation of providing a plurality of nucleic acid template molecules immobilized on a support. Each nucleic acid template molecule may comprise an insert sequence of interest. The insert sequence can be different in different template molecules. Each template molecule may correspond to a polony of optical signals in flow cell images.

Before the operation of 610, the method 600 may comprise an operation of generating flow cell images by conducting one or more cycles of sequencing reactions of the plurality of nucleic acid template molecules immobilized on the support. The flow cell images can be generated or acquired by the sequencing system disclosed herein. Conducting the one or more cycles of the sequencing reactions may comprise contacting the plurality of nucleotide acid template molecules using a plurality of nucleotide reagents comprising a mixture of different types of nucleotide bases A, G, C and T/U. Individual nucleotide reagent may comprise a different detectable color label that corresponds with each different type of nucleotide base.

In some embodiments, conducting the one or more cycles of the sequencing reactions may comprise: contacting the plurality of nucleotide acid template molecules with a plurality of sequencing primers, a plurality of polymerases and a mixture of different types of avidites. An individual avidite in the mixture may comprise a core attached with multiple nucleotide arms and each arm of the individual avidite comprises the same type of nucleotide base. In some embodiments, conducting the one or more cycles of the sequencing reactions comprises: in each of the one or more cycles, imaging optical color signals emitted from nucleotide reagents that are bound to the plurality of template molecules. Imaging the optical signals may be performed by an optical system, e.g., the imager 116, disclosed herein. In some embodiments conducting the one or more cycles of the sequencing reactions may comprise: in each of the one or more cycles, acquiring the flow cell images comprising optical color signals emitted from nucleotide reagents that are bound to the plurality of template molecules.

In some embodiments, the flow cell images comprises optical signals emitted from nucleotide reagents bound to a unbalanced diversity of nucleotide bases of A, G, C and T/U among the plurality of nucleic acid template molecules immobilized on the support in the one or more cycles. In some embodiments, the plurality of polonies comprise a unbalanced diversity of nucleotide bases of A, G, C and T/U, and wherein the unbalanced diversity comprises a percentage of: (1) a number of one or more types of nucleotide bases to (2) a total number of nucleotide bases, and the percentage is less than 20%, 15%, 10%, or 5% in the cycle N.

In some aspects, the method 600 may include an operation 620 of determining corrected image intensities of the plurality of polonies in cycle N, Ipc(N) based on the phasing coefficient, pN−1, the prephasing coefficient, ppN−1, and image intensities of the plurality of polonies in cycle N, I(N). In operations 610, the phasing and/or prephasing contaminations to the image intensity I(N) is removed using phasing and/or prephasing coefficient from the previous cycle N−1. This phasing and/or prephasing correction is preliminary since the coefficients for the current cycle N are not available yet, and the preliminary correction may be updated in subsequent steps in method 600. The corrected image intensities of the plurality of polonies in cycle N, Ipc(N), may be calculated using equation (1).

In some aspects, the method 600 may include an operation 630 of iterating one or more of the operations 631-635 (illustrated in FIG. 6B) until a stopping criterion is met. The first iteration of operations 631-635 may be similar to method 500 shown in FIG. 5. The second iteration of operation 631-635 may start with the corrected image intensities, Ipc_n(N), from the first iteration, and use the base calling made in the previous iteration to select polonies that needs phasing and/or prephasing correction and perform normalization before base calling in the second iteration.

In some aspects, the method 600 may include an operation 631 of determining, by the processor, corrected image intensities of the plurality of polonies in cycle N+1, Ipc(N+1). This operation 631 is similar to operation 510 when pN and/or ppN is not available, for example, in the first iteration of method 600. Ipc(N+1) may be calculated using equation (2).

In some aspects where pN and/or ppN is available, e.g., has been calculated from a previous iteration, Ipc(N+1) may be determined based on the phasing coefficient, pN; the prephasing coefficient, ppN; and image intensities of the plurality of polonies in cycle N or cycle N+1, I(N+1), using equation (3).

The method 600 may further comprise an operation of generating normalized image intensities, Inorm(N), of cycle N, and Inorm(N+1) as described in method 500.

The method 600 may further comprise an operation 632 of obtaining, by the processor, base calls in cycle N based on the corrected image intensities of the plurality of polonies in cycle N, Ipc(N).

The method 600 may further comprise an operation of loading, receiving, or making the base calls in cycle N−1 as described in method 500.

The method 600 may further comprise operations 633, 634, and 635 that are similar to operations 530, 540, and 550, respectively. The method 600 may further comprise other operations that have been disclosed herein in relation to method 500.

In some aspects, a method for performing phasing and prephasing correction on image intensity in cycle N, which is the current cycle, instead of using intensities from N−1, N, N+1, and N+2, may use image intensities from more previous cycles and subsequent cycles, e.g., N−2, N−1, N, N+1, N+2, and N+3.

In some aspects, most of the operations in the method 600 may be identical to the operations described in method 500. The operations that are different from those in method 500 are described below.

In some aspects, the method may include an operation of determining the corrected image intensities in cycle N, Ipc(N), for each polony as:

$${I}_{p c} ( N ) = I ( N ) - {p}_{N - 1} * I ( N - 1 ) - {p}_{N - 1}^{′} ⋆ ( N - 2 ) - {pp}_{N - 1} * I ( N + 1 ) - {pp}_{N - 1}^{′} ( N + 2 ) \qquad ( 5 )$$

where pN−1 is the phasing coefficient of cycle N−1, ppN−1 is the prephasing coefficient of cycle N−1, p′N−1 is a second phasing coefficient of cycle N−1, pp′N−1 is a second prephasing coefficient of cycle N−1, I(N+2) is the image intensity of the polony in cycle N+2, and I(N−2) is the intensity of the same polony in cycle N−2.

In some aspects, the corrected image intensities in cycle N, Ipc(N+1), for each polony may be obtained as:

$${I}_{p c} ( N + 1 ) = I ( N + 1 ) - {p}_{N - 1} * I ( N ) - {p}_{N - 1}^{′} * ( N - 1 ) - {pp}_{N - 1} * I ( N + 2 ) - {pp}_{N - 1}^{′} ( N + 3 ) \qquad ( 6 )$$

where pN−1 is the phasing coefficient of cycle N−1, ppN−1 is the prephasing coefficient of cycle N−1, p′N−1 is a second phasing coefficient of cycle N−1, pp′N−1 is a second prephasing coefficient of cycle N−1.

In some aspects, there may be an operation of updating the image intensities of the plurality of polonies in cycle N, I(N), using updated and corrected image intensities, Ipc_n(N). In some aspects, this operation may be performed by the FPGAs.

The updated and corrected image intensity, Ipc_n(N), may be obtained as:

$${I}_{pc \_} ( N ) = I ( N ) - {p}_{N} * I ( N - 1 ) - {p}_{N} I ( N - 2 ) - p {p}_{N} * I ( N + 1 ) - p {p}_{N} I ( N + 2 ) \qquad ( 7 )$$

wherein pN is the phasing coefficient of cycle N, ppN is the prephasing coefficient of cycle N, p′N is a second phasing coefficient of cycle N, pp′N is a second prephasing coefficient of cycle N−1.

In some aspects, the second coefficients p′N and pp′N may be determined similarly as in operation 530 but by selecting polonies that are “on” in cycle N−2, but “off” in cycle N and the polonies that are “on” in cycle N+2, but “off” in cycle N. After such selection, or fitting of the selected polonies may be used to calculate the second coefficients p′N and pp′N.

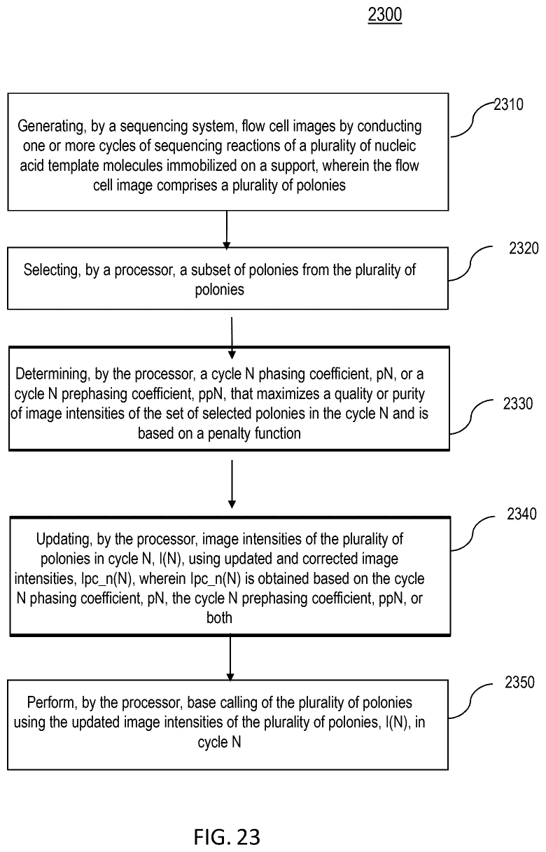

FIG. 23 illustrates a flow chart of a method for performing phasing and prephasing corrections, according to some aspects. The method 2300 may include some or all of the operations disclosed herein. The operations may be performed in the order that is described herein, but is not limited to the order that has been described herein.

The method 2300 may include determining values of the phasing and/or prephasing coefficients, pN and/or ppN, among candidate coefficient values based on penalty function(s) for one or more sequencing cycles. The methods 2300 may include performing phasing and prephasing corrections on flow cell images of cycle N using different candidate coefficient values; after such corrections, determining quality or purity sum using image intensities corresponding to the different candidate coefficient values and based on penalty value(s) determined by the penalty function(s); selecting the coefficient values that maximize the quality or purity sum as the phasing and prephasing coefficients; and utilizing the phasing and prephasing coefficient values for image intensity correction of cycle N. The correction of the image intensity can be performed using equation (4) herein. The image intensity of polonies or clusters after phasing and prephasing correction may be used for base calling with improved accuracy and reliability in comparison with base calls using image intensities without such correction.

The method 2300 may be performed by one or more processors disclosed herein. In some aspects, the processor may include one or more of: a processing unit, an integrated circuit, or their combinations. For example, the processing unit may include a central processing unit (CPU) and/or a graphic processing unit (GPU). The integrated circuit may include a chip such as a field-programmable gate array (FPGA). In some aspects, the processor may include the computing system 400.

In some aspects, some or all operations in method 2300 may be performed by the FPGAs. In aspects when some operations are performed by FPGAs, the data after an operation performed by the FPGA may be communicated by the FPGAs to the CPUs so that CPUs may perform subsequent operation(s) in method 2300 using such data. In some aspects, all the operations in methods herein may be performed by CPUs. Alternatively, the operations performed by CPUs may be performed by other processors such as the dedicated processors, or GPUs.

The methods 500, 600, 2300 may advantageously improve base calling accuracy and reliability over base callings without phasing and prephasing correction or with insufficient phasing and prephasing corrections. The methods 500, 600, 2300 may advantageously improve base calling accuracy and reliability where the phased and prephased polonies or clusters are above a specific density (e.g., greater than 10² per mm²) and/or percentage over a total number of polonies or clusters on the support (e.g., greater than 10% or 15% of the total number of polonies or clusters).

The methods 500, 600, 2300 may allow correction of phasing and prephasing of a plurality of polonies or clusters even if the polonies or clusters are of low or unbalanced diversity in sequencing cycle(s). The nucleotide diversity of a population of immobilized polonies or clusters can refer to the relative proportion of nucleotides A, G, C and T/U that are present in each sequencing cycle. An optimal high diversity library can generally include approximately equal proportions of all four types of nucleotides represented in each cycle of a sequencing run. A low diversity library can generally include a high proportion of certain nucleotide types and a low proportion of other nucleotide types in one or more sequencing cycles.

The balanced diversity of nucleotide bases of A, G, C and T/U among the plurality of nucleic acid template molecules can comprises: a percentage of (1) a number of each type of nucleotide bases to (2) a total number of bases in the one or more cycles. The percentage can be more than about 8%, 10%, 12%, 15%, 18%, 20%, or 22%. For example, the balanced diversity of nucleotide bases includes a number of nucleotide bases A, G, C, T that is 26%, 15%, 27%, and 32% respectively of the total number of all nucleotide bases among the polonies of a sequencing cycle.

In some embodiments, the polonies or clusters being sequenced in a flow cycle may have a certain nucleotide diversity. The nucleotide diversity of a population of nucleotide acid molecules, e.g., polonies or clusters, can refer to the relative proportion of nucleotides A, G, C, and T/U that are present in each flow cycle. An optimally high or balanced diversity data can generally have approximately equal proportions of all four nucleotides represented in each flow cycle of a sequencing run. A low or unbalanced diversity data can generally include a high proportion of certain nucleotides and a low proportion of other nucleotides in some flow cycles of a sequencing run, e.g., less than 10% of the total number of all 4 nucleotides. As a result, images corresponding to the high portion of certain nucleotides can have a greater number of brighter spots (polonies) than images corresponding to the low portion of certain nucleotides. As an example of low or unbalanced diversity data in a flow cycle, the bases A, T, C, G can be about 1%, about 2%, about 1%, and about 95%, respectively, of the total number of polonies, in a certain flow cycle. As another example of low or unbalanced diversity data, the bases A, T, C, G in polonies at multiple flow cycles can be about 2%, about 5%, about 10%, and about 83%, respectively. In embodiments where low or unbalanced diversity data is present in a sequencing run and is imaged for sequencing analysis, image registration failure may occur because image(s) from one or more channels are too dark (e.g., signal spots of polonies are too sparse and/or dim) comparing with images acquired from other channels.

In addition to the base biases affecting diversity, plexity can also be a factor that when plexity is lower than a number, e.g., 8 or 16, the signal could be of low diversity. For example, in a 2-cycle sequence, all polonies are of AT or TG or GC or CA. It is 25% for every base in every cycle, but its plexity is less than 8, and the sequence is not all random. In some embodiments, the methods 500 is configured to register flow cell images even if the polonies are of low diversity or low plexity.

In general, plexity can indicate source(s) of the sample. A uniplex sample may include DNA fragments or molecules from a same sample region in a genome or a same sample source. A multiplex sample may include DNA fragments or molecules from different sample sources, e.g., liver, kidney, heart, cancerous tissue, etc, or from one or more sample regions in the genome.

In some aspects, the method 2300 is performed during a cycle N, so that base calling of cycles prior to cycle N (e.g., cycle N−1) has already been performed, while base calling of cycle N (and similarly, cycle N+1, N+2) is yet to be performed. In some aspects, cycle N is the current cycle. While sequencing of the current cycle N is being performed, the base calls of cycles prior to cycle N may have been saved to a memory or a data storage device disclosed herein. The base calls of cycles prior to cycle N may be loaded from the memory or data storage device. N may be any integer that is greater than 2. For example, for short read sequencing, N may be any integer from 2 to 150.

In some aspects, the method 2300 may include an operation 2310 of generating flow cell images by conducting one or more cycles of sequencing reactions of a plurality of nucleic acid template molecules immobilized on a support. The plurality of nucleic acid template molecules may be of a 2D sample immobilized on the support. The flow cell image may comprise a plurality of polonies that corresponds to the template molecules. The operation 2310 may be performed by the imaging system disclosed herein.

Before the operation 2310, the method 2300 may comprise an operation of providing a plurality of nucleic acid template molecules immobilized on the support. Each nucleic acid template molecule may comprise an insert sequence of interest. The insert sequence can be different in different template molecules. Each template molecule may correspond to a polony of optical signals in flow cell images.

In some embodiments, conducting the one or more cycles of the sequencing reactions may comprise contacting the plurality of nucleotide acid template molecules using a plurality of nucleotide reagents comprising a mixture of different types of nucleotide bases A, G, C and T/U. An individual nucleotide reagent may comprise a different detectable color label that corresponds with each different type of nucleotide base.

In some embodiments, conducting the one or more cycles of the sequencing reactions may comprise: contacting the plurality of nucleotide acid template molecules with a plurality of sequencing primers, a plurality of polymerases and a mixture of different types of avidites. An individual avidite in the mixture may comprise a core attached with multiple nucleotide arms and each arm of the individual avidite comprises the same type of nucleotide base.

In some embodiments, conducting the one or more cycles of the sequencing reactions comprises: in each of the one or more cycles, imaging optical color signals emitted from nucleotide reagents that are bound to the plurality of template molecules. Imaging the optical signals may be performed by an optical system, e.g., the imager 116, disclosed herein. In some embodiments conducting the one or more cycles of the sequencing reactions may comprise: in each of the one or more cycles, acquiring the flow cell images comprising optical color signals emitted from nucleotide reagents that are bound to the plurality of template molecules.

In some embodiments, the flow cell images comprises optical signals emitted from nucleotide reagents bound to a unbalanced diversity of nucleotide bases of A, G, C and T/U among the plurality of nucleic acid template molecules immobilized on the support in the one or more cycles. In some embodiments, the plurality of polonies comprise a unbalanced diversity of nucleotide bases of A, G, C and T/U, and wherein the unbalanced diversity comprises a percentage of: (1) a number of one or more types of nucleotide bases to (2) a total number of nucleotide bases, and the percentage is less than 20%, 15%, 10%, or 5% in the cycle N.

In some embodiments, the methods 2300 include an operation 2310 of generating the flow cell images from 3D cellular sample(s) by conducting one or more cycles of sequencing reactions of a plurality of concatemer molecules of a cellular sample immobilized on the support. Among the concatemer molecules, a first concatemer molecule of the plurality of concatemer molecules may correspond to a first target RNA molecule of the cellular sample, and a second concatemer molecule of the plurality of concatemer molecules may correspond to a second target RNA molecule of the cellular sample. The flow cell image may comprise a plurality of polonies corresponding to the plurality of concatemer molecules of the cellular sample. In some embodiments, the cellular sample may comprise one or more in situ samples. For example, the cellular sample comprises one or more cells or tissue. The cellular sample may extend in an axial direction (i.e., z direction) orthogonal to the image plane of the flow cell images so that flow cell images at different axial locations are required to cover the 3D volume of the cellular sample. In such embodiments of in situ sample, the flow cell images are acquired at a plurality of predetermined axial locations that are spaced apart from each other along an axial direction orthogonal to an image plane of the flow cell images to include signal from multiple 2D image planes or a 3D volume. Each of the plurality of concatemer molecules immobilized on the support may correspond to a polony. Each of the plurality of concatemer molecules immobilized on the support corresponds to a base calling location. The plurality of predetermined axial locations comprises 3 to 500 predetermined axial locations. Each of the plurality of predetermined axial locations are spaced 1, 2, 3, 4, 5, 6, 7, 8, 9, 10, 11, 12, 13, 14, or 15 m apart from an adjacent neighboring axial location thereof. Each of the plurality of predetermined axial locations are spaced 0.1 to 400 μm apart from an adjacent neighboring axial location thereof.

Before the operation 2310, the method 2300 may comprise an operation of providing a plurality of template molecules (e.g., concatemer molecules) of a cellular sample immobilized on the support. Each concatemer molecule may correspond to a target RNA molecule of the cellular sample. In some embodiments, each concatemer molecule comprises tandem repeat units, wherein a unit comprises a sequence that corresponds to a target cDNA (or the target RNA), a target barcode sequence, and a universal sequencing primer binding site (or a complementary sequence thereof). Each concatemer molecule may correspond to a polony of optical signals in flow cell images.

In some embodiments, conducting the one or more cycles of the sequencing reactions may comprise contacting the plurality of concatemer template molecules using a plurality of nucleotide reagents comprising a mixture of different types of nucleotide bases A, G, C and T/U. An individual nucleotide reagent may comprise a different detectable color label that corresponds with each different type of nucleotide base.

In some embodiments, conducting the one or more cycles of the sequencing reactions may comprise: contacting the plurality of concatemer molecules with a plurality of sequencing primers, a plurality of polymerases and a mixture of different types of avidites. An individual avidite in the mixture may comprise a core attached with multiple nucleotide arms and each arm of the individual avidite comprises the same type of nucleotide base.

In some embodiments, conducting the one or more cycles of the sequencing reactions comprises: in each of the one or more cycles, imaging optical color signals emitted from nucleotide reagents that are bound to the plurality of concatemer molecules. Imaging the optical signals may be performed by an optical system, e.g., the imager 116, disclosed herein. In some embodiments conducting the one or more cycles of the sequencing reactions may comprise: in each of the one or more cycles, acquiring the flow cell images comprising optical color signals emitted from nucleotide reagents that are bound to the plurality of concatemer molecules.

In some embodiments, the methods 2300 may comprise an operation 2320 of selecting a subset of polonies from the plurality of polonies.

In some aspects, the method 2300 is configured to correct phasing and prephasing of a plurality of polonies or clusters by selecting a subset of polonies therefrom. The subset of polonies or clusters may be extracted (e.g., their locations and/or corresponding intensities may be identified and saved in a list disclosed herein) from flow cell images acquired from one or more channels. For example, the plurality of polonies may be extracted from flow cell images from 4 different channels of a specific sequencing cycle. As another example, the plurality of polonies may be extracted from flow cell images from a single channel. An example technique for extracting the polonies in a flow cell is described in U.S. Pat. No. 11,200,446, which is hereby incorporated by reference in its entirety. The flow cell image as disclosed herein can be an image that is acquired using a support, e.g., a flow cell 112 as shown in FIG. 1. In some embodiments, the subset of polonies or clusters may be extracted from specific regions of a tile, e.g., each subtile. Within each subtile, the polonies may be extracted with a predetermined pattern or randomly.

In some embodiments, the subset of polonies are selected to ensure that different regions of the flow cell images (e.g., subtiles) and/or different intensities (e.g., relatively brighter and darker regions) are substantially included in the selection. In some embodiments, the subset of polonies may be much smaller in number than the plurality of polonies, e.g., 2%, 5%, 10%, 20%, 30%, or 40% of the plurality of polonies. In some embodiments, the subset of polonies may be selected using various down-sampling techniques. In some embodiments, the subset of polonies are selected to ensure that the phase and prephasing correction of all the polonies of the subset may substantially represent phasing and phasing correction that is needed at individual polonies or clusters. For example, the subset of polonies may be selected to be within a spatial boundary when it is predetermined that the level of phasing and prephasing may have a spatial dependency across a tile or different tiles.

In some embodiments, selecting the subset of polonies from the plurality of polonies is based on base calls in the cycle N−1 and/or cycle N−2. In some embodiments, selecting the subset of polonies from the plurality of polonies comprises: for each channel of one or more channels: selecting polonies that are called a base corresponding to the channel in the cycle N and called a different base in the cycle N−1; selecting polonies that are called a base corresponding to the channel in the cycle N and called a different base in the cycle N+1; or a combination thereof.

In some embodiments, the subset of selected polonies comprises polonies randomly selected from multiple subtiles. The plurality of nucleic acid template molecules may be immobilized at random locations on the support. The plurality of nucleic acid template molecules may be immobilized at pre-determined locations on the support.

In some embodiments, the method 2300 may comprise an operation 2330 of determining a value of a cycle N phasing coefficient, pN, a value of a cycle N prephasing coefficient, ppN, or both that maximizes a quality function of image intensities of the subset of selected polonies in the cycle N and is based on a penalty function. In some embodiments, the quality function may be the quality score or purity of image intensities of the subset of selected polonies. In some embodiments, the quality score or purity of image intensities of the subset of selected polonies comprises: a purity sum that is generated by adding up corresponding purities of individual polonies of the subset of selected polonies; or a quality sum generated by adding up corresponding quality scores of individual polonies of the subset of selected polonies.

In some embodiments, the operation 2330 comprises: generating multiple non-repetitive set of candidate values, each set of candidate values includes a candidate value of the cycle N phasing coefficient, pN, and a candidate value of the cycle N prephasing coefficient, ppN. In some embodiments, the candidate coefficients can be selected from predetermined ranges. The predetermined ranges may be obtained using various methods. For example, the predetermined ranges may be determined empirically corresponding to the sequencing system, flow cell, sequencing process, and imaging parameters. As an example, the candidate coefficient may be within 0 to 0.99%. As another example, the candidate coefficient may be within 0.1% to 0.6%, 0 to 0.5%, 0 to 0.6%, or other ranges within the range of 0 to 1%. The number of candidate coefficients can vary. Having a greater number of candidate values, e.g., 1000 sets of candidates, may require more computational resources and/or longer time for determining the coefficients. When the number gets big enough, e.g., 200 sets of candidates, it may delay the determination thereby making real-time base calling difficult to be achieved (e.g., generating base calls of cycle N before the sequencing reactions in cycle N+2 is completed). As an example, 5 different candidates are determined for each coefficient, and there can be 25 non-repetitive combinations of candidate phasing and prephasing coefficients. In this example, pN or ppN can be 0, 0.2%, 0.4%, 0.6%, or 0.8%. In this example, pN or ppN can be 0, 0.1%, 0.2%, 0.4%, or 0.7%. Alternatively, pN or ppN can be a number of candidate coefficient(s) selected from the range of 0 to 1%.

In some embodiments, the operation 2330 further comprises: determining a candidate purity or quality sum for each of the non-repetitive sets of candidates. The candidate purity or quality sum may be a sum of individual purity or quality from each of the selected polonies determined using a non-repetitive set of candidate coefficients.

The operation of determining the candidate purity or quality sum for each of the non-repetitive sets of candidates may comprise: determining individual purity or quality of each of the set of selected polonies. In some embodiments, determining individual purity or quality of each polony of the set of selected polonies comprises: generating a penalty shift of at least some of the image intensities in response to determining that the at least some of the image intensities are below a predetermined threshold; and determining individual purity or quality of each polony of the set of selected polonies based on the shifted image intensities. An exemplary predetermined threshold can be 0. The penalty shift can be identical for the subset of polonies across different channels within a same cycle. The penalty shift can vary dynamically in different cycles. In some embodiments, the penalty shift can be predetermined so that it remains the same for different polonies across channels and/or in multiple cycles.

In some embodiments, determining individual purity or quality of each polony of the set of selected polonies comprises: adding a first penalty constant to a first image intensity and a second penalty constant to a second image intensity. The adding operation may be in response to determining that the at least some of the image intensities are below a predetermined threshold. In some embodiments, determining individual purity or quality of each polony of the set of selected polonies further comprises: determining individual purity or quality of each of the set of selected polonies based on the shifted image intensities. The first and second penalty constants may be identical for the selected polonies within a single cycle. In some embodiments, the first and second penalty constants can be predetermined so that it remains the same for different polonies across channels and/or in multiple cycles.

As an example, individual purity at polony i can be calculated as:

$$purity ( i ) = [ Max ( i ) + c ] / [ Max ( i ) + SecondMax ( i ) + b ]$$

where Max(i) represents maximum channel intensity among all the channels for polony i, and SecondMax(i) represents a second maximum channel intensity among all the channels for polony I, and where c and b are predetermined positive constants. c and b can both be 1 in some embodiments. The maximum channel intensity and the second maximum channel intensity can be obtained after at least some of the preprocessing operations disclosed herein has been performed, e.g., normalization of signal intensity across channels.

For low or unbalanced diversity samples, some polonies may have low image intensities from certain channels (e.g., image intensity that is approximately 0 or comparable to background noise). Such low image intensities may be below a predetermined threshold after phasing and/or prephasing correction. Adding the first penalty constant and/or the second penalty constant can be advantageous in shifting the low image intensities so that they are not below the predetermined threshold thereby providing more accuracy and reliability in base calling of the low or unbalanced diversity sample.

In some embodiments, the operation of determining a candidate purity or quality sum for each of the non-repetitive sets of candidates comprises: adding together all the individual purity, purity(i), of each polony of the subset of the selected polonies.

In some embodiments, the operation 2330 further comprises: subtracting a penalty value from the candidate quality sum or purity sum corresponding to each of the non-repetitive sets of candidates. The penalty value may be determined using the penalty function disclosed herein, thereby generating a corresponding adjusted candidate sum for each of the non-repetitive sets of candidates.

For each non-repetitive set of a candidate value of the cycle N phasing coefficient, pN, and a candidate value of the cycle N prephasing coefficient, ppN, the operation 2330 comprise: determining the penalty value using the penalty function based on a first value of the candidate cycle N phasing coefficient, pN, a second value of the candidate cycle N prephasing coefficient, ppN, or both the first and second values. The penalty function may generate a penalty value that increases in response to an increase in a sum of the first and second value of the pN and ppN, respectively. In such embodiments, the first and second values correspond to a set of the non-repetitive sets of candidates. The penalty function may generate a penalty value that decreases in response to a decrease in a sum of the first and second value of the pN and ppN. The penalty function, pf, disclosed herein may be various functions that penalize larger phasing and/or prephasing coefficients over smaller coefficients so that it advantageously ensures more accurate and reliable phasing and prephasing correction especially for low diversity or unbalance diversity data. For example, the penalty function, pf, can be calculated as pf=(pN+ppN)*C, where C is a positive constant. As another example, the penalty function, pf, can be calculated as pf=(pN²+ppN²)*C, or pf=[√(pN²+ppN²)]*C, where C is a positive constant.

In some embodiments, the operation 2330 further comprises: determining the candidate value of the cycle N phasing coefficient, pN, and the candidate value of cycle N prephasing coefficient, ppN, that maximize the corresponding adjusted candidate sum as the value of the cycle N phasing coefficient, pN, and the value of the cycle N prephasing coefficient.

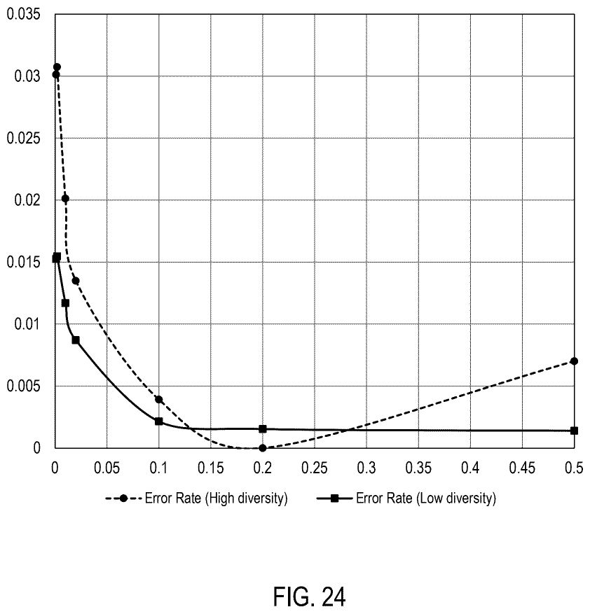

In some embodiments, an error rate in base calling is decreased by using the penalty function in comparison with base calling without using the penalty function disclosed herein. FIG. 24 shows a comparison of error rates for a low diversity sample and a high diversity data sample using the phasing and prephasing correction method disclosed herein. The penalty function is pf=(pN+ppN)*C. The samples each include about 2 millions of polonies per tile and a total number of 6-12 tiles, and are each sequenced in 150 cycles. The low diversity sample has at least one type of nucleotide bases that is below 10% of the total number of bases. The high diversity sample has each type of nucleotide bases above 10% of the total number of bases. The horizontal axis represents values of the constant C. Each of the phasing and prephasing coefficient can be one of the five different values: 0, 0.2%, 0.4%, 0.6%, or 0.8%. Therefore, the phasing and prephasing coefficients have 25 non-repetitive set of candidate values. The vertical axis represents error rates in base calling after phasing and prephasing correction using the methods disclosed herein. The error rates in base calls of the low diversity data is similar or better than the error rates in base calls of the high diversity data across different values of the constant, C.

In some embodiments, the plurality of polonies comprise an unbalanced diversity of nucleotide bases in the cycle N. In some embodiments, the plurality of polonies comprise an unbalanced diversity of nucleotide bases in the cycle N−1, N, N+1, or their combinations.

In some embodiments, the quality score is proportional to a logarithm of an error rate of base calling.

In some embodiments, the support is passivated with at least one hydrophilic polymer coating having a water contact angle of not more than 45 degrees. The at least one hydrophilic polymer coating may comprise a molecule selected from a group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N−isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. The at least one hydrophilic polymer coating may comprise branched hydrophilic polymer molecules having at least four branches. In some embodiments, the at least one hydrophilic polymer coating comprises polymer molecules having a molecular weight of at least 1000 Daltons.

In some embodiments, a density of the nucleic acid template molecules on the support is 10⁴-10¹² per mm². In some embodiments, a density of the nucleic acid template molecules on the support is 10⁴-10⁸ per mm². In some embodiments, a sample source of the sample herein is genomic DNA, double-stranded cDNA or cell free circulating DNA. Two or more different immobilized template molecules may have different insert sequences.

In some embodiments, the method 2300 comprises an operation of performing one or more preprocessing steps on the image intensities of the plurality of polonies in the cycles N−1, N, N+1, N+2, or the combination thereof. The one or more preprocessing steps may comprise: background subtraction; image sharpening; intensity extraction; intensity offset adjustment; color correction; intensity normalization across different channels within a same cycle or image normalization across multiple cycles; or a combination thereof. The one or more preprocessing steps may be similar as those for methods 500, 600, and 2300.

In some embodiments, the operation of performing the one or more preprocessing steps occurs before determining the cycle N phasing coefficient, pN, the cycle N prephasing coefficient, ppN, or both. In some embodiments, performing the one or more preprocessing steps is before selecting the subset of polonies from the plurality of polonies in the cycle N.

In some embodiments, performing the one or more preprocessing steps is by one or more FPGAs. In some embodiments, the methods 2300 further comprises communicating data (intermediary data for generating base calls) between the FPGAs and one or more CPUs.

The method 2300 may comprise an operation 2340 of updating the image intensities of the plurality of polonies in cycle N, I(N), using updated and corrected image intensities, Ipc_n(N). The operation 2340 may comprise: generating, by the processor, the updated and corrected image intensities, Ipc_n(N).

In some embodiments, the operation 2340 may comprise: generating normalized image intensities, Inorm(N), by normalizing the image intensities, I(N); and generating the updated and corrected image intensities, Ipc_n(N), by correcting the normalized image intensities, Inorm(N), with the cycle N phasing coefficient, pN, the cycle N prephasing coefficient, ppN, or both the cycle N phasing coefficient and the cycle N prephasing coefficient, ppN, that maximizes a quality or purity of image intensities of the set of selected polonies in the cycle N. Normalizing the image intensities in cycle N, I(N), may comprise dividing the image intensities in cycle N by a normalization factor. The normalization factor may be an image intensity selected from a range of intensity among at least part of the polonies that are called the base corresponding to the channel. Updating the image intensities of the plurality of polonies in cycle N, I(N), using updated and corrected image intensities, Ipc_n(N) may comprises: generating, by the processor, the updated and corrected image intensities, Ipc_n(N).

In alternative embodiments, generating the updated and corrected image intensities, Ipc_n(N) may comprise: generating the corrected image intensities, Ipc_c(N), by correcting the image intensities, I(N), with the cycle N phasing coefficient, pN, the cycle N prephasing coefficient, ppN, or both the cycle N phasing coefficient and the cycle N prephasing coefficient, ppN; generating normalized image intensities, Inorm(N); and generating the updated and corrected image intensities, by normalizing the image intensities, Ipc_c(N).

In some embodiments, the corrected image intensities in cycle N, Ipc_n(N), is further based on image intensities of the plurality of polonies in cycle N−1, I(N−1). In some embodiments, the corrected image intensities in cycle N, Ipc_n(N), is further based on image intensities of the plurality of polonies in cycle N+1, I(N+1). In some embodiments, the updated and corrected image intensities, Ipc_n(N), are from one or more channels. In some embodiments, the updated and corrected image intensities, Ipc_n(N), are from 2, 3, or 4 channels.

In some embodiments, the plurality of polonies is selected from flow cell images from multiple channels. The plurality of polonies is selected from flow cell images from 4 channels. In some embodiments, the plurality of polonies is selected from flow cell images from a single channel.

### Example Computer System

FIG. 4 illustrates a block diagram of a computer system for phasing and prephasing correction, according to some aspects. Various aspects of the methods described herein, such as methods 500 and 600, as well as combinations and sub-combinations thereof, may be implemented, for example, using one or more computer systems, such as computer system 400 shown in FIG. 4.

Computer system 400 may include one or more hardware processors 404. The hardware processor 404 may include a central processing unit (CPU), graphic processing units (GPU), or their combination. Processor 404 may be connected to a bus or communication infrastructure 406.

Computer system 400 may also include user input/output device(s) 403, such as monitors, keyboards, pointing devices, etc., which may communicate with communication infrastructure 406 through user input/output interface(s) 402. The user input/output devices 403 may be coupled to the user interface 124 in FIG. 1.

One or more of processors 404 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning aspects described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

Computer system 400 may also include a data storage device such as a main or primary memory 408, e.g., random access memory (RAM). Main memory 408 may include one or more levels of cache. Main memory 408 may have stored therein control logic (i.e., computer software) and/or data.

Computer system 400 may also include one or more secondary data storage devices or secondary memory 410. Secondary memory 410 may include, for example, a main storage drive 412 and/or a removable storage device or drive 414. Main storage drive 412 may be a hard disk drive or solid-state drive, for example. Removable storage drive 414 may be a floppy disk drive, a magnetic tape drive, a compact disk drive, an optical storage device, tape backup device, and/or any other storage device/drive.

Removable storage drive 414 may interact with a removable storage unit 418.

Removable storage unit 418 may include a computer usable or readable storage device having stored thereon computer software and/or data. The software may include control logic. The software may include instructions executable by the hardware processor(s) 404. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 400. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 may further include a communication or network interface 424. Communication interface 424 may enable computer system 400 to communicate and interact with any combination of external devices, external networks, external entities, etc. (individually and collectively referenced by reference number 428). For example, communication interface 424 may allow computer system 400 to communicate with external or remote devices 428 over communication path 426, which may be wired and/or wireless (or a combination thereof), and which may include any combination of LANs, WANs, the Internet, etc. Control logic and/or data may be transmitted to and from computer system 400 via communication path 426. In some aspects, communication path 426 is the connection to the cloud 130, as depicted in FIG. 1. The external devices, etc. referred to by reference number 428 may be devices, networks, entities, etc. in the cloud 130.

Computer system 400 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device.

Computer system 400 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some aspects, a tangible, non-transitory apparatus or article of manufacture comprising a tangible, non-transitory computer usable or readable medium having control logic (software) stored thereon may also be referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), may cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 4. In particular, aspects may operate with software, hardware, and/or operating system implementations other than those described herein.

### Imaging Modules and Systems

Returning to FIG. 1, the imager 116 may include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some aspects, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being imaged.

In some aspects, improvements in imaging performance, e.g., for dual-side (flow cell) imaging applications comprising the use of thick flow cell walls (e.g., wall (or coverslip) thickness>700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some aspects, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary aspects disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 m, at least 500 m, at least 600 m, at least 700 m, at least 800 am, at least 900 m, at least 1000 m, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some aspects, the numerical aperture may be at least 0.75. In some aspects, the numerical aperture is at least 1.0. In some aspects, the working distance is at least 850 m. In some aspects, the working distance is at least 1,000 am. In some aspects, the field-of-view may have an area of at least 2.5 mm². In some aspects, the field-of-view may have an area of at least 3 mm². In some aspects, the spatial sampling frequency may be at least 2.5 times the optical resolution of the fluorescence imaging system. In some aspects, the spatial sampling frequency may be at least 3 times the optical resolution of the fluorescence imaging system. In some aspects, the system may further comprise an X-Y-Z translation stage such that the system is configured to acquire a series of two or more fluorescence images in an automated fashion, wherein each image of the series is or may be acquired for a different field-of-view. In some aspects, a position of the sample plane may be simultaneously adjusted in an X direction, a Y direction, and a Z direction to match the position of an objective lens focal plane in between acquiring images for different fields-of-view. In some aspects, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some aspects, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some aspects, the specified error threshold is 100 nm or greater. In some aspects, the specified error threshold is 50 nm or less. In some aspects, the system comprises three or more image sensors, and wherein the system is configured to image fluorescence in each of three or more wavelength ranges onto a different image sensor. In some aspects, a difference in the position of a focal plane for each of the three or more image sensors and the sample plane is less than 100 nm. In some aspects, a difference in the position of a focal plane for each of the three or more image sensors and the sample plane is less than 50 nm. In some aspects, the total time required to reposition the sample plane, adjust focus if necessary, and acquire an image is less than 0.4 seconds per field-of-view. In some aspects, the total time required to reposition the sample plane, adjust focus if necessary, and acquire an image is less than 0.2 seconds per field-of-view.

Also disclosed herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some aspects, the objective lens may be a commercially-available microscope objective. In some aspects, the commercially-available microscope objective may have a numerical aperture of at least 0.3. In some aspects, the objective lens may have a working distance of at least 700 μm. In some aspects, the objective lens may be corrected to compensate for a cover slip thickness (or flow cell wall thickness) of 0.17 mm or of greater or lesser thickness than 0.17 mm. In some aspects, the optical system may be corrected to compensate for cover slip thickness, flow cell thickness, or distance between desired focal planes. In some aspects, said correction may be made by inserting a corrective optic, such as a lens or optical assembly into the light path of the optical system. In some aspects, said correction may be made without inserting a corrective optic, such as a lens or optical assembly into the light path of the optical system.

In some aspects, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some aspects, the at least one tube lens may be a compound lens comprising three or more optical components. In some aspects, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some aspects, the at least one tube lens is configured to correct an imaging performance metric for a combination of the objective lens, the at least one tube lens, and the at least one image sensor when imaging an interior surface of a flow cell having a wall thickness of at least 1 mm.

In some aspects, the at least one tube lens is configured to correct an imaging performance metric for a combination of the objective lens, the at least one tube lens, and the at least one image sensor when imaging an interior surface of a flow cell having a gap of at least 100 μm. In some aspects, the at least one tube lens is configured to correct an imaging performance metric for a combination of the objective lens, the at least one tube lens, and the at least one image sensor when imaging an interior surface of a flow cell having a gap of at least 200 μm. In some aspects, the system comprises a single objective lens, two tube lenses, and two image sensors, and each of the two tube lenses is designed to provide optimal imaging performance at a different fluorescence wavelength. In some aspects, the system comprises a single objective lens, three tube lenses, and three image sensors, and each of the three tube lenses is designed to provide optimal imaging performance at a different fluorescence wavelength. In some aspects, the system comprises a single objective lens, four tube lenses, and four image sensors, and each of the four tube lenses is designed to provide optimal imaging performance at a different fluorescence wavelength. In some aspects, the design of the objective lens or the at least one tube lens is configured to optimize the modulation transfer function in the mid to high spatial frequency range. In some aspects, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof.

In some aspects, the difference in the imaging performance metric for imaging the upper interior surface and the lower interior surface of the flow cell is less than 10%. In some aspects, the difference in imaging performance metric for imaging the upper interior surface and the lower interior surface of the flow cell is less than 5%. In some aspects, the use of the at least one tube lens provides for an at least equivalent or better improvement in the imaging performance metric for dual-side imaging compared to that for a conventional system comprising an objective lens, a motion-actuated compensator, and an image sensor. In some aspects, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some aspects, the illumination system further comprises a condenser lens. In some aspects, the specified field-of-illumination has an area of at least 2 mm². In some aspects, the light delivered to the specified field-of-illumination is of uniform intensity across a specified field-of-view for an imaging system used to acquire images of the support surface. In some aspects, the specified field-of-view has an area of at least 2 mm². In some aspects, the light delivered to the specified field-of-illumination is of uniform intensity across the specified field-of-view when a coefficient of variation (CV) for light intensity is less than 10%. In some aspects, the light delivered to the specified field-of-illumination is of uniform intensity across the specified field-of-view when a coefficient of variation (CV) for light intensity is less than 5%. In some aspects, the light delivered to the specified field-of-illumination has a speckle contrast value of less than 0.1. In some aspects, the light delivered to the specified field-of-illumination has a speckle contrast value of less than 0.05.

It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectic focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

Aspects of the present disclosure provide methods for sequencing immobilized or non-immobilized template molecules. The methods may be operated in system 100, for example, in sequencer 114. In some aspects, the immobilized template molecules comprise a plurality of nucleic acid template molecules having one copy of a target sequence of interest. In some aspects, nucleic acid template molecules having one copy of a target sequence of interest may be generated by conducting bridge amplification using linear library molecules. In some aspects, the immobilized template molecules comprise a plurality of nucleic acid template molecules each having two or more tandem copies of a target sequence of interest (e.g., concatemers). In some aspects, nucleic acid template molecules comprising concatemer molecules may be generated by conducting rolling circle amplification of circularized linear library molecules. In some aspects, the non-immobilized template molecules comprise circular molecules. In some aspects, methods for sequencing employ soluble (e.g., non-immobilized) sequencing polymerases or sequencing polymerases that are immobilized to a support.

In some aspects, the sequencing reactions employ detectably labeled nucleotide analogs. In some aspects, the sequencing reactions employ a two-stage sequencing reaction comprising binding detectably labeled multivalent molecules, and incorporating nucleotide analogs. In some aspects, the sequencing reactions employ non-labeled nucleotide analogs. In some aspects, the sequencing reactions employ phosphate chain labeled nucleotides.

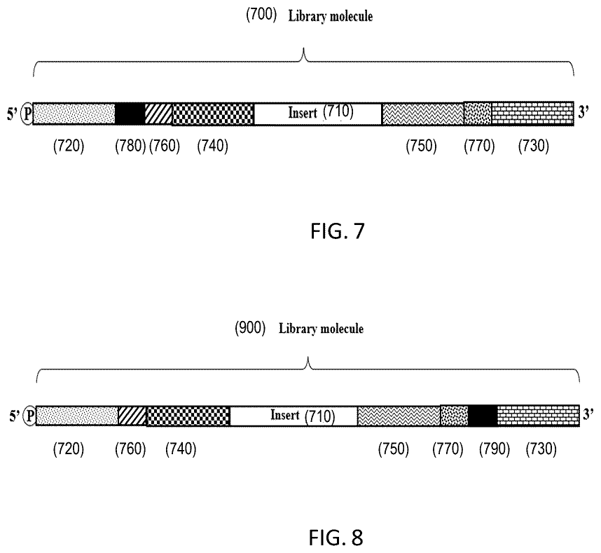

In some aspects, the immobilized concatemers each comprise tandem repeat units of the sequence-of-interest (e.g., insert region) and any adaptor sequences. For example, the tandem repeat unit comprises: (i) a left universal adaptor sequence having a binding sequence for a first surface primer (920) (e.g., surface pinning primer), (ii) a left universal adaptor sequence having a binding sequence for a first sequencing primer (940) (e.g., forward sequencing primer), (iii) a sequence-of-interest (910), (iv) a right universal adaptor sequence having a binding sequence for a second sequencing primer (950) (e.g., reverse sequencing primer), (v) a right universal adaptor sequence having a binding sequence for a second surface primer (930) (e.g., surface capture primer), and (vii) a left sample index sequence (960) and/or a right sample index sequence (970). In some aspects, the tandem repeat unit further comprises a left unique identification sequence (980) and/or a right unique identification sequence (990). In some aspects, the tandem repeat unit further comprises at least one binding sequence for a compaction oligonucleotide. In some aspects, FIGS. 7 and 8 show linear library molecules or a unit of a concatemer molecule.

Specifically, FIG. 7 is a schematic showing an exemplary linear single stranded library molecule 700, according to some aspects, which comprises: a surface pinning primer binding site (720); an optional left unique identification sequence (780); a left index sequence (760); a forward sequencing primer binding site (740); an insert region having a sequence of interest (710); reverse sequencing primer binding site (750); a right index sequence (770); and a surface capture primer binding site (730).

FIG. 8 is a schematic showing an exemplary linear single stranded library molecule (900), according to some aspects, which comprises: a surface pinning primer binding site (720); a left index sequence (760); a forward sequencing primer binding site (740); an insert region having a sequence of interest (710); a reverse sequencing primer binding site (750); a right index sequence (770); an optional right unique identification sequence (790); and a surface capture primer binding site (730), according to some aspects.

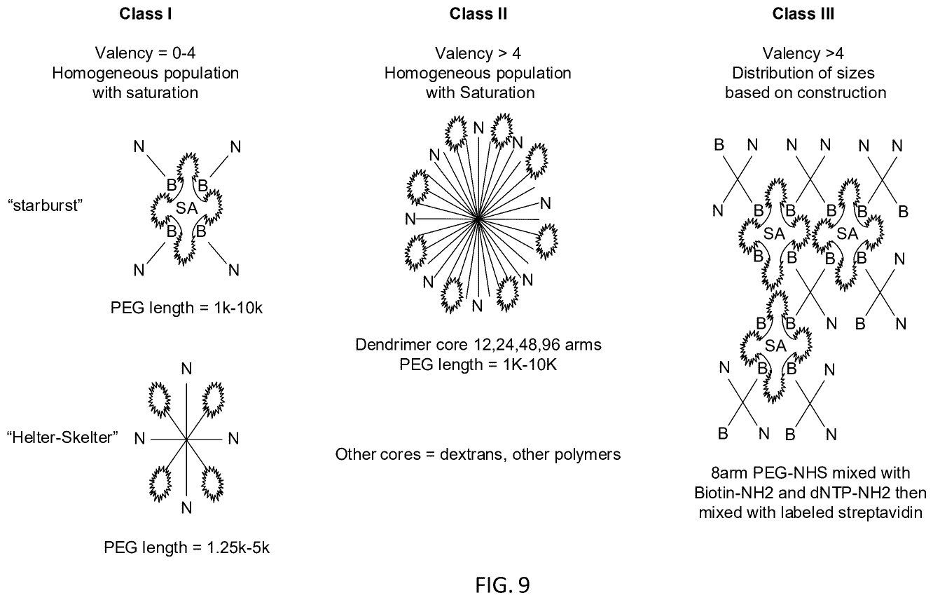

FIG. 9 is a schematic of various exemplary configurations of multivalent molecules. Left (Class I): schematics of multivalent molecules having a “starburst” or “helter-skelter” configuration, according to some aspects. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

The immobilized concatemer may self-collapse into a compact nucleic acid nanoball. Inclusion of one or more compaction oligonucleotides during the rolling circle amplification (RCA) reaction may further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer may be simultaneously sequenced. Furthermore, a plurality of binding complexes may form along a particular concatemer molecule, each binding complex comprising a sequencing polymerase bound to a template/primer duplex and bound to a multivalent molecule, wherein the plurality of binding complexes remain stable without dissociation resulting in increased persistence time which increases signal intensity and reduces imaging time.

### Methods for Sequencing Using Nucleotide Analogs

Aspects of the present disclosure provide methods for sequencing any of the immobilized template molecules described herein, the methods comprising step (a): contacting a sequencing polymerase to (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some aspects, the sequencing polymerase comprises a recombinant mutant sequencing polymerase that may bind and incorporate nucleotide analogs.

In some aspects, in the methods for sequencing template molecules, the sequencing primer comprises a 3′ extendible end or a 3′ non-extendible end. In some aspects, the plurality of nucleic acid template molecules comprise amplified template molecules (e.g., clonally amplified template molecules). In some aspects, the plurality of nucleic acid template molecules comprise one copy of a target sequence of interest. In some aspects, the plurality of nucleic acid molecules comprise two or more tandem copies of a target sequence of interest (e.g., concatemers). In some aspects, the plurality of nucleic acid template molecules comprise the same target sequence of interest or different target sequences of interest. In some aspects, the plurality of nucleic acid primers are in solution or are immobilized to a support. In some aspects, when the plurality of nucleic acid template molecules and/or the plurality of nucleic acid primers are immobilized to a support, the binding with the first sequencing polymerase generates a plurality of immobilized first complexed polymerases. In some aspects, the plurality of nucleic acid template molecules and/or nucleic acid primers are immobilized to 10²-10¹⁵ different sites on a support. In some aspects, the binding of the plurality of template molecules and nucleic acid primers with the plurality of first sequencing polymerases generates a plurality of first complexed polymerases immobilized to 102-10¹⁵ different sites on the support. In some aspects, the plurality of immobilized first complexed polymerases on the support are immobilized to pre-determined or to random sites on the support. In some aspects, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some aspects, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some aspects, the sequencing polymerase is contacted with the plurality of nucleotides in the presence of at least one catalytic cation comprising magnesium and/or manganese. In some aspects, the plurality of nucleotides comprises at least one nucleotide analog having a chain terminating moiety at the sugar 2′ or 3′ position. In some aspects, the chain terminating moiety is removable from the sugar 2′ or 3′ position to convert the chain terminating moiety to an OH or H group. In some aspects, the plurality of nucleotides comprises at least one nucleotide that lacks a chain terminating moiety. In some aspects, at least one nucleotide is labeled with a detectable reporter moiety (e.g., fluorophore) that emits a detectable signal. The detectable reporter moiety comprises a fluorophore. In some aspects, the fluorophore is attached to the nucleo-base. In some aspects, the fluorophore is attached to the nucleo-base with a linker which is cleavable/removable from the base. In some aspects, at least one of the nucleotides in the plurality is not labeled with a detectable reporter moiety. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some aspects, step (b) further comprises identifying the nucleo-based of the incorporated chain terminating nucleotide.

In some aspects, the methods for sequencing further comprise step (c): removing the chain terminating moiety from the incorporated chain terminating nucleotide to generate an extendible 3′OH group. In some aspects, step (c) further comprises removing the detectable label from the incorporated chain terminating nucleotide. In some aspects, the sequencing polymerase remains bound to the template molecule which is hybridized to the sequencing primer which is extended by one nucleo-base. In some aspects, the methods for sequencing further comprise step (d): repeating steps (b) and (c) at least once in order to build out the nucleotide sequence.

### Methods for Nucleic Acid Sequencing

Aspects of the present disclosure provide a method for sequencing any of the immobilized template molecules described herein. In some aspects, the first portion generally comprises binding multivalent molecules to complexed polymerases to form multivalent-complexed polymerases, and detecting the multivalent-complexed polymerases.

In some aspects, the first portion comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some aspects, the first polymerase comprises a recombinant mutant sequencing polymerase.

In some aspects, in the methods for sequencing template molecules, the sequencing primer comprises an oligonucleotide having a 3′ extendible end or a 3′ non-extendible end. In some aspects, the plurality of nucleic acid template molecules comprise amplified template molecules (e.g., clonally amplified template molecules). In some aspects, the plurality of nucleic acid template molecules comprise one copy of a target sequence of interest. In some aspects, the plurality of nucleic acid molecules comprise two or more tandem copies of a target sequence of interest (e.g., concatemers). In some aspects, the nucleic acid template molecules in the plurality of nucleic acid template molecules comprise the same target sequence of interest or different target sequences of interest. In some aspects, the plurality of nucleic acid template molecules and/or the plurality of nucleic acid primers are in solution or are immobilized to a support. In some aspects, when the plurality of nucleic acid template molecules and/or the plurality of nucleic acid primers are immobilized to a support, the binding with the first sequencing polymerase generates a plurality of immobilized first complexed polymerases. In some aspects, the plurality of nucleic acid template molecules and/or nucleic acid primers are immobilized to 10²-10¹⁵ different sites on a support. In some aspects, the binding of the plurality of template molecules and nucleic acid primers with the plurality of first sequencing polymerases generates a plurality of first complexed polymerases immobilized to 10²-10¹⁵ different sites on the support. In some aspects, the plurality of immobilized first complexed polymerases on the support are immobilized to pre-determined or to random sites on the support. In some aspects, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

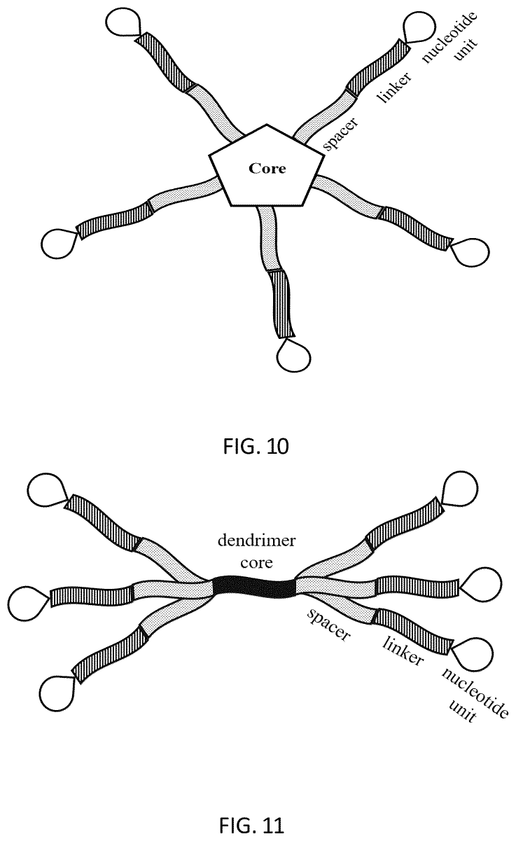

In some aspects, the methods for sequencing further comprise step (b): contacting the plurality of first complexed polymerases with a plurality of multivalent molecules to form a plurality of multivalent-complexed polymerases (e.g., binding complexes). In some aspects, individual multivalent molecules in the plurality of multivalent molecules comprise a core attached to multiple nucleotide arms and each nucleotide arm is attached to a nucleotide (e.g., nucleotide unit) (e.g., FIGS. 10-14). In some aspects, the contacting of step (b) is conducted under a condition suitable for binding complementary nucleotide units of the multivalent molecules to at least two of the plurality of first complexed polymerases thereby forming a plurality of multivalent-complexed polymerases. In some aspects, the condition is suitable for inhibiting polymerase-catalyzed incorporation of the complementary nucleotide units into the primers of the plurality of multivalent-complexed polymerases. In some aspects, the plurality of multivalent molecules comprises at least one multivalent molecule having multiple nucleotide arms (e.g., FIGS. 10-13) each attached with a nucleotide analog (e.g., nucleotide analog unit), where the nucleotide analog includes a chain terminating moiety at the sugar 2′ and/or 3′ position. In some aspects, the plurality of multivalent molecules comprises at least one multivalent molecule comprising multiple nucleotide arms each attached with a nucleotide unit that lacks a chain terminating moiety. In some aspects, at least one of the multivalent molecules in the plurality of multivalent molecules is labeled with a detectable reporter moiety that emits a signal. In some aspects, the detectable reporter moiety comprises a fluorophore. In some aspects, the contacting of step (b) is conducted in the presence of at least one non-catalytic cation comprising strontium, barium and/or calcium.

In some aspects, the methods for sequencing further comprise step (c): detecting the plurality of multivalent-complexed polymerases. In some aspects, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some aspects, the multivalent molecules are labeled with a detectable reporter moiety to permit detection. In some aspects, the labeled multivalent molecules comprise a fluorophore attached to the core, linker and/or nucleotide unit of the multivalent molecules.

In some aspects, the methods for sequencing further comprise step (d): identifying the nucleo-base of the complementary nucleotide units that are bound to the plurality of first complexed polymerases, thereby determining the sequence of the template molecule. In some aspects, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some aspects, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some aspects, the second portion of the sequencing method generally comprises nucleotide incorporation. In some aspects, the methods for sequencing further comprise step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some aspects, the second sequencing polymerase comprises a recombinant mutant sequencing polymerase.

In some aspects, the plurality of first sequencing polymerases of step (a) have an amino acid sequence that is 100% identical to the amino acid sequence as the plurality of the second sequencing polymerases of step (f). In some aspects, the plurality of first sequencing polymerases of step (a) have an amino acid sequence that differs from the amino acid sequence of the plurality of the second sequencing polymerases of step (f).

In some aspects, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some aspects, the contacting of step (g) is conducted under a condition that is suitable for promoting polymerase-catalyzed incorporation of the bound complementary nucleotides into the primers of the nucleotide-complexed polymerases thereby extending the sequencing primer by one nucleo-base. In some aspects, the incorporating the nucleotide into the 3′ end of the sequencing primer in step (g) comprises a primer extension reaction. In some aspects, the contacting of step (g) is conducted in the presence of at least one catalytic cation comprising magnesium and/or manganese. In some aspects, the plurality of nucleotides comprise native nucleotides (e.g., non-analog nucleotides) or nucleotide analogs. In some aspects, the plurality of nucleotides comprise a 2′ and/or 3′ chain terminating moiety which is removable or is not removable. In some aspects, at least one of the nucleotides in the plurality is not labeled with a detectable reporter moiety. In some aspects, the plurality of nucleotides are non-labeled. In some aspects, the plurality of nucleotides comprises a plurality of nucleotides labeled with detectable reporter moiety. The detectable reporter moiety comprises a fluorophore. In some aspects, the fluorophore is attached to the nucleotide base. In some aspects, the fluorophore is attached to the nucleotide base with a linker which is cleavable/removable from the base or is not removable from the base. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleotide base.

In some aspects, when the plurality of nucleotides in step (g) are detectably labeled, the methods for sequencing further comprise step (h): detecting the complementary nucleotides which are incorporated into the primers of the nucleotide-complexed polymerases. In some aspects, the plurality of nucleotides are labeled with a detectable reporter moiety to permit detection. In some aspects, when the plurality of nucleotides in step (g) are non-labeled, the detecting of step (h) is omitted.

In some aspects, when the plurality of nucleotides in step (g) are detectably labeled, the methods for sequencing further comprise step (i): identifying the bases of the complementary nucleotides which are incorporated into the primers of the nucleotide-complexed polymerases. In some aspects, the identification of the incorporated complementary nucleotides in step (i) may be used to confirm the identity of the complementary nucleotides of the multivalent molecules that are bound to the plurality of first complexed polymerases in step (d). In some aspects, the identifying of step (i) may be used to determine the sequence of the nucleic acid template molecules. In some aspects, when the plurality of nucleotides in step (g) are non-labeled, the identifying of step (i) is omitted.

In some aspects, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some aspects, the methods for sequencing further comprise step (k): repeating steps (a)-(j) at least once. In some aspects, the sequence of the nucleic acid template molecules may be determined by detecting and identifying the multivalent molecules that bind the sequencing polymerases but do not incorporate into the 3′ end of the primer at steps (c) and (d). In some aspects, the sequence of the nucleic acid template molecule may be determined (or confirmed) by detecting and identifying the nucleotide that incorporates into the 3′ end of the primer at steps (h) and (i).

In some aspects, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some aspects, the first sequencing polymerase comprises any wild type or mutant polymerase described herein. In some aspects, the second sequencing polymerase comprises any wild type or mutant polymerase described herein. The concatemer template molecule comprises tandem repeat sequences of a sequence of interest and at least one universal sequencing primer binding site. The first and second nucleic acid primers may bind to a sequencing primer binding site along the concatemer template molecule. Exemplary multivalent molecules are shown in FIGS. 10-13.

In some aspects, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some aspects, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers may bind to a sequencing primer binding site along the concatemer template molecule. Exemplary multivalent molecules are shown in FIGS. 10-13.

### Sequencing-by-Binding

Aspects of the present disclosure provide methods for sequencing any of the immobilized template molecules described herein, wherein the sequencing methods comprise a sequencing-by-binding (SBB) procedure which employs non-labeled chain-terminating nucleotides. In some aspects, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (the contents of both patents are hereby incorporated by reference in their entireties).

### Methods for Sequencing Using Phosphate-Chain Labeled Nucleotides

Aspects of the present disclosure provide methods for sequencing using immobilized sequencing polymerases which bind non-immobilized template molecules, wherein the sequencing reactions are conducted with phosphate-chain labeled nucleotides. In some aspects, the sequencing methods comprise step (a): providing a support having a plurality of sequencing polymerases immobilized thereon. In some aspects, the sequencing polymerase comprises a processive DNA polymerase. In some aspects, the sequencing polymerase comprises a wild type or mutant DNA polymerase, including for example a Phi29 DNA polymerase. In some aspects, the support comprises a plurality of separate compartments and a sequencing polymerase is immobilized to the bottom of a compartment. In some aspects, the separate compartments comprise a silica bottom through which light may penetrate. In some aspects, the separate compartments comprise a silica bottom configured with a nanophotonic confinement structure comprising a hole in a metal cladding film (e.g., aluminum cladding film). In some aspects, the hole in the metal cladding has a small aperture, for example, approximately 70 nm. In some aspects, the height of the nanophotonic confinement structure is approximately 100 nm. In some aspects, the nanophotonic confinement structure comprises a zero mode waveguide (ZMW). In some aspects, the nanophotonic confinement structure contains a liquid.

In some aspects, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some aspects, the individual sequencing primers hybridize to a universal sequencing primer binding site on the single stranded circular template molecule.

In some aspects, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups may be referred to as alpha, beta and gamma phosphate groups. In some aspects, a particular detectable reporter moiety which is attached to the terminal phosphate group corresponds to the nucleotide base (e.g., dATP, dGTP, dCTP, dTTP or dUTP) to permit detection and identification of the nucleo-base. In some aspects, the plurality of polymerase/template/primer complexes are contacted with the plurality of phosphate chain labeled nucleotides under a condition suitable for polymerase-catalyzed nucleotide incorporation. In some aspects, the sequencing polymerases are capable of binding a complementary phosphate chain labeled nucleotide and incorporating the complementary nucleotide opposite a nucleotide in a template molecule. In some aspects, the polymerase-catalyzed nucleotide incorporation reaction cleaves between the alpha and beta phosphate groups thereby releasing a multi-phosphate chain linked to a fluorophore.

In some aspects, the sequencing method further comprises step (d): detecting the fluorescent signal emitted by the phosphate chain labeled nucleotide that is bound by the sequencing polymerase, and incorporated into the terminal end of the sequencing primer. In some aspects, step (d) further comprises identifying the phosphate chain labeled nucleotide that is bound by the sequencing polymerase, and incorporated into the terminal end of the sequencing primer.

In some aspects, the sequencing method further comprises step (d): repeating steps (c)-(d) at least once. In some aspects, sequencing methods that employ phosphate chain labeled nucleotides may be conducted according to the methods described in U.S. Pat. Nos. 7,170,050; 7,302,146; and/or 7,405,281.

### Sequencing Polymerases

Aspects of the present disclosure provide methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some aspects, the sequencing polymerase(s) is/are capable of incorporating a complementary nucleotide opposite a nucleotide in a template molecule. In some aspects, the sequencing polymerase(s) is/are capable of binding a complementary nucleotide unit of a multivalent molecule opposite a nucleotide in a template molecule. In some aspects, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Nucleotides

Aspects of the present disclosure provide methods for sequencing nucleic acid molecules, where any of the sequencing methods described herein employ at least one nucleotide. The nucleotides comprise a base, sugar and at least one phosphate group. In some aspects, at least one nucleotide in the plurality comprises an aromatic base, a five carbon sugar (e.g., ribose or deoxyribose), and one or more phosphate groups (e.g., 1-10 phosphate groups). The plurality of nucleotides may comprise at least one type of nucleotide selected from a group consisting of dATP, dGTP, dCTP, dTTP and dUTP. The plurality of nucleotides may comprise at least a mixture of any combination of two or more types of nucleotides selected from a group consisting of dATP, dGTP, dCTP, dTTP and/or dUTP. In some aspects, at least one nucleotide in the plurality is not a nucleotide analog. In some aspects, at least one nucleotide in the plurality comprises a nucleotide analog.

In some aspects, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some aspects, at least one nucleotide in the plurality is an analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

In some aspects, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some aspects, the chain terminating moiety may inhibit polymerase-catalyzed incorporation of a subsequent nucleotide unit or free nucleotide in a nascent strand during a primer extension reaction. In some aspects, the chain terminating moiety is attached to the 3′ sugar position where the sugar comprises a ribose or deoxyribose sugar moiety. In some aspects, the chain terminating moiety is removable/cleavable from the 3′ sugar position to generate a nucleotide having a 3′OH sugar group which is extendible with a subsequent nucleotide in a polymerase-catalyzed nucleotide incorporation reaction. In some aspects, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some aspects, the chain terminating moiety is cleavable/removable from the nucleotide, for example by reacting the chain terminating moiety with a chemical agent, pH change, light or heat. In some aspects, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the chain terminating moieties aryl and benzyl are cleavable with H2 Pd/C. In some aspects, the chain terminating moieties amine, amide, keto, isocyanate, phosphate, thio, disulfide are cleavable with phosphine or with a thiol group including beta-mercaptoethanol or dithiothritol (DTT). In some aspects, the chain terminating moiety carbonate is cleavable with potassium carbonate (K₂CO₃) in MeOH, with triethylamine in pyridine, or with Zn in acetic acid (AcOH). In some aspects, the chain terminating moieties urea and silyl are cleavable with tetrabutylammonium fluoride, pyridine-HF, with ammonium fluoride, or with triethylamine trihydrofluoride. In some aspects, the chain terminating moiety may be cleavable/removable with nitrous acid. In some aspects, a chain terminating moiety may be cleavable/removable using a solution comprising nitrite, such as, for example, a combination of nitrite with an acid such as acetic acid, sulfuric acid, or nitric acid. In some further aspects, said solution may comprise an organic acid.

In some aspects, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some aspects, the chain terminating moiety comprises an azide, azido or azidomethyl group. In some aspects, the chain terminating moiety comprises a 3′-O-azido or 3′-O-azidomethyl group. In some aspects, the chain terminating moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl)phosphine (TCEP) or bis-sulfo triphenyl phosphine (BS-TPP) or Tri(hydroxyproyl)phosphine (THPP). In some aspects, the cleaving agent comprises 4-dimethylaminopyridine (4-DMAP). In some aspects, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some aspects, the chain terminating moiety comprising one or more of a 3′-O-amino group, a 3′-O-aminomethyl group, a 3′-O-methylamino group, or derivatives thereof may be cleaved using a solution comprising nitrite. In some aspects, for example, nitrite may be combined with or contacted with an acid such as acetic acid, sulfuric acid, or nitric acid. In some further aspects, for example, nitrite may be combined with or contacted with an organic acid such as, for example, formic acid, acetic acid, propionic acid, butyric acid, isobutyric acid, or the like. In some aspects, the chain terminating moiety comprises a 3′-acetal moiety which may be cleaved with a palladium deblocking reagent (e.g., Pd(0)).

In some aspects, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some aspects, in any of the methods for sequencing nucleic acid molecules described herein, the plurality of nucleotides comprises a plurality of nucleotides labeled with detectable reporter moiety. The detectable reporter moiety comprises a fluorophore. In some aspects, the fluorophore is attached to the nucleotide base. In some aspects, the fluorophore is attached to the nucleotide base with a linker which is cleavable/removable from the base. In some aspects, at least one of the nucleotides in the plurality is not labeled with a detectable reporter moiety. In some aspects, a particular detectable reporter moiety (e.g., fluorophore) that is attached to the nucleotide may correspond to the nucleotide base (e.g., dATP, dGTP, dCTP, dTTP or dUTP) to permit detection and identification of the nucleotide base.

In some aspects, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some aspects, the cleavable linker on the base is cleavable/removable from the base by reacting the cleavable moiety with a chemical agent, pH change, light or heat. In some aspects, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the cleavable moieties aryl and benzyl are cleavable with H2 Pd/C. In some aspects, the cleavable moieties amine, amide, keto, isocyanate, phosphate, thio, disulfide are cleavable with phosphine or with a thiol group including beta-mercaptoethanol or dithiothritol (DTT). In some aspects, the cleavable moiety carbonate is cleavable with potassium carbonate (K₂CO₃) in MeOH, with triethylamine in pyridine, or with Zn in acetic acid (AcOH). In some aspects, the cleavable moieties urea and silyl are cleavable with tetrabutylammonium fluoride, pyridine-HF, with ammonium fluoride, or with triethylamine trihydrofluoride.

In some aspects, in any of the methods for sequencing nucleic acid molecules described herein, the cleavable linker on the nucleotide base comprises cleavable moiety including an azide, azido or azidomethyl group. In some aspects, the cleavable moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl)phosphine (TCEP) or bis-sulfo triphenyl phosphine (BS-TPP) or Tri(hydroxyproyl)phosphine (THPP). In some aspects, the cleaving agent comprises 4-dimethylaminopyridine (4-DMAP).

In some aspects, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some aspects, the chain terminating moiety (e.g., at the sugar 2′ and/or sugar 3′ position) and the detectable reporter moiety linked to the base are chemically cleavable/removable with the same chemical agent. In some aspects, the chain terminating moiety (e.g., at the sugar 2′ and/or sugar 3′ position) and the detectable reporter moiety linked to the base are chemically cleavable/removable with different chemical agents.

### Multivalent Molecules

Aspects of the present disclosure provide methods for sequencing nucleic acid molecules, where any of the sequencing methods described herein employ at least one multivalent molecule. In some aspects, the multivalent molecule comprises a plurality of nucleotide arms attached to a core and having any configuration including a starburst, helter skelter, or bottle brush configuration (e.g., FIG. 10). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some aspects, the nucleotide unit comprises a base, sugar and at least one phosphate group, and the linker is attached to the nucleotide unit through the base. In some aspects, the linker comprises an aliphatic chain or an oligo ethylene glycol chain where both linker chains having 2-6 subunits. In some aspects, the linker also includes an aromatic moiety.

FIG. 10 is a schematic of an exemplary multivalent molecule comprising a generic core attached to a plurality of nucleotide arms, according to some aspects.

FIG. 11 is a schematic of an exemplary multivalent molecule comprising a dendrimer core attached to a plurality of nucleotide arms, according to some aspects.

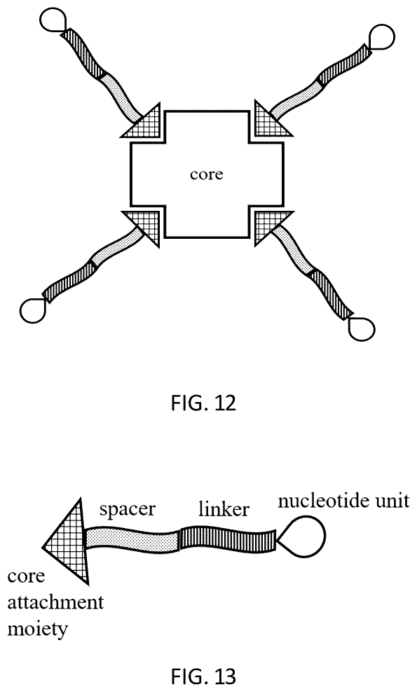

FIG. 12 shows a schematic of an exemplary multivalent molecule comprising a core attached to a plurality of nucleotide arms, where the nucleotide arms comprise biotin, spacer, linker and a nucleotide unit, according to some aspects.

FIG. 13 is a schematic of an exemplary nucleotide arm comprising a core attachment moiety, spacer, linker and nucleotide unit, according to some aspects.

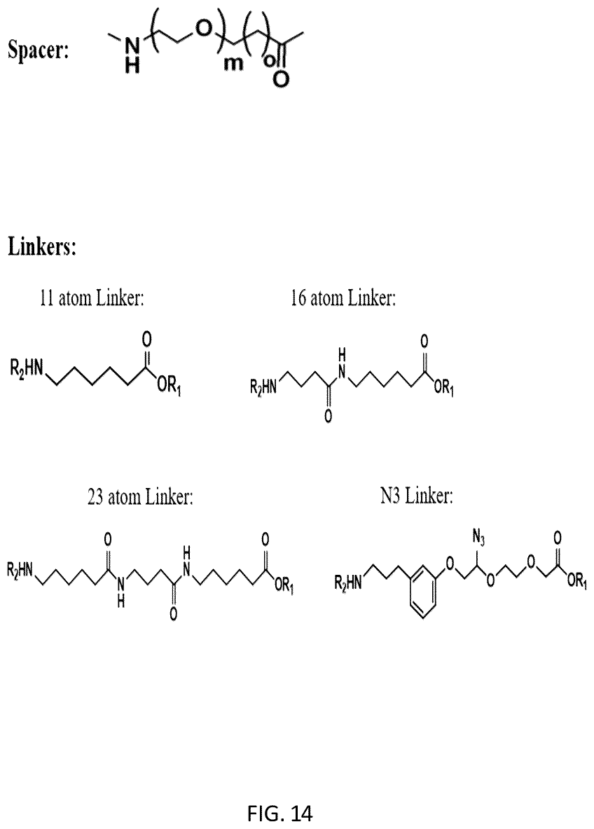

FIG. 14 shows the chemical structure of an exemplary spacer (top), and the chemical structures of various exemplary linkers, including an 11-atom Linker, 16-atom Linker, 23-atom Linker and an N3 Linker (bottom), according to some aspects.

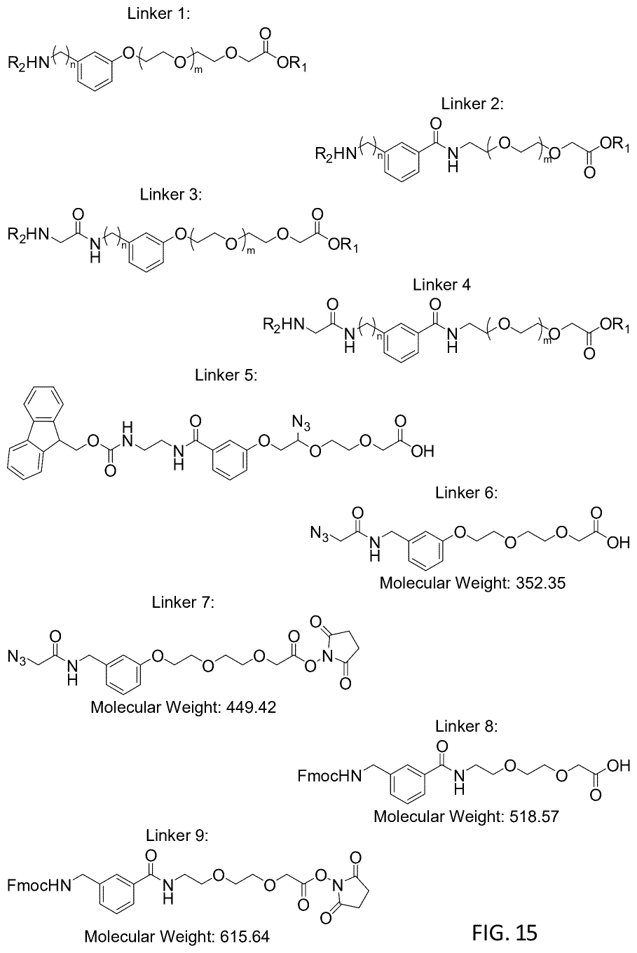

FIG. 15 shows the chemical structures of various exemplary linkers, including Linkers 1-9, according to some aspects.

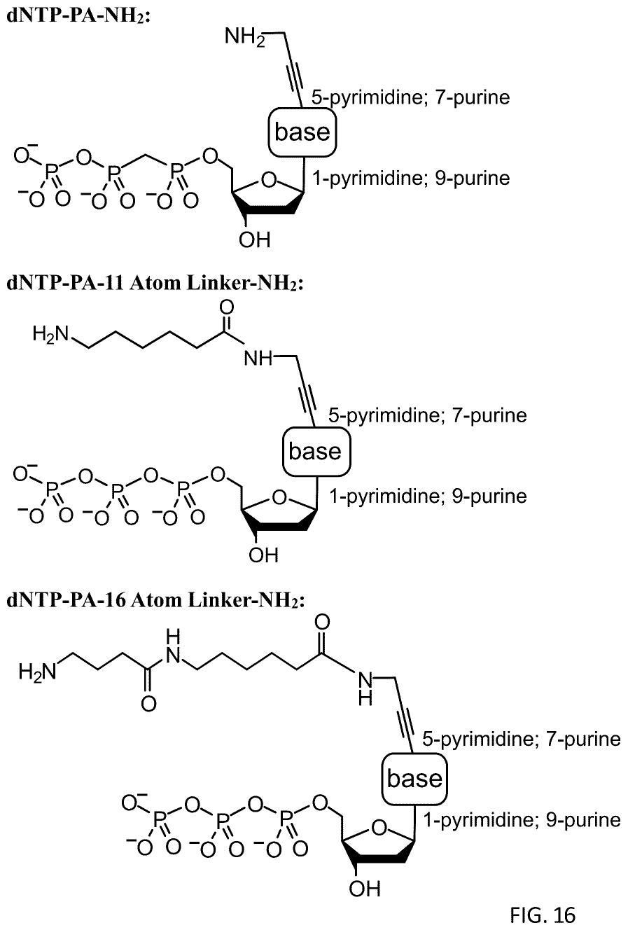

FIG. 16 shows the chemical structures of various exemplary linkers joined/attached to nucleotide units, according to some aspects.

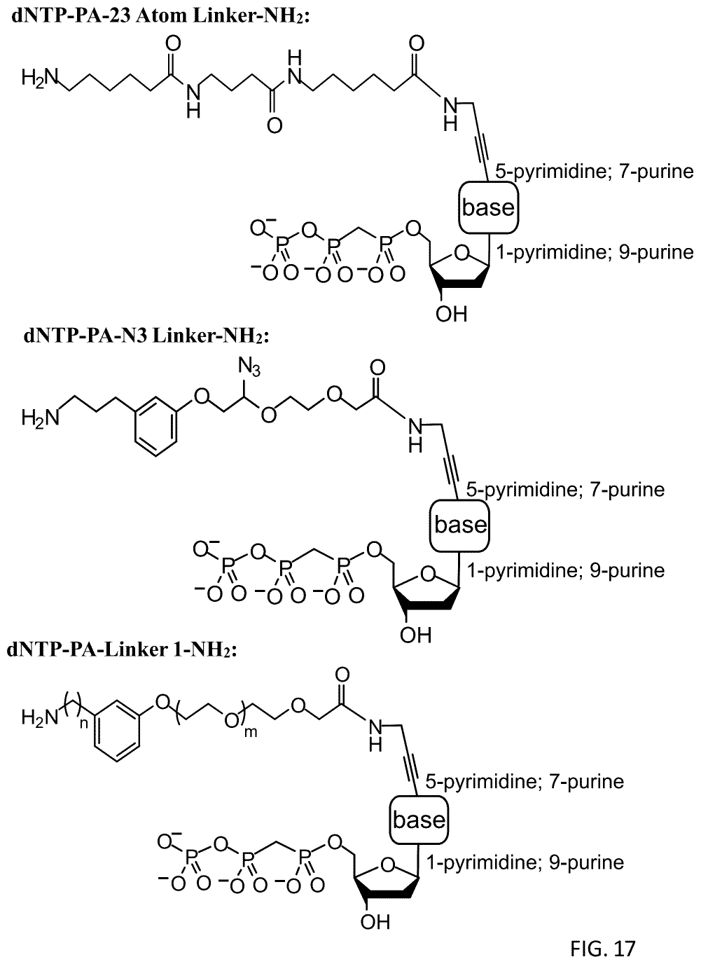

FIG. 17 shows the chemical structures of various exemplary linkers joined/attached to nucleotide units, according to some aspects.

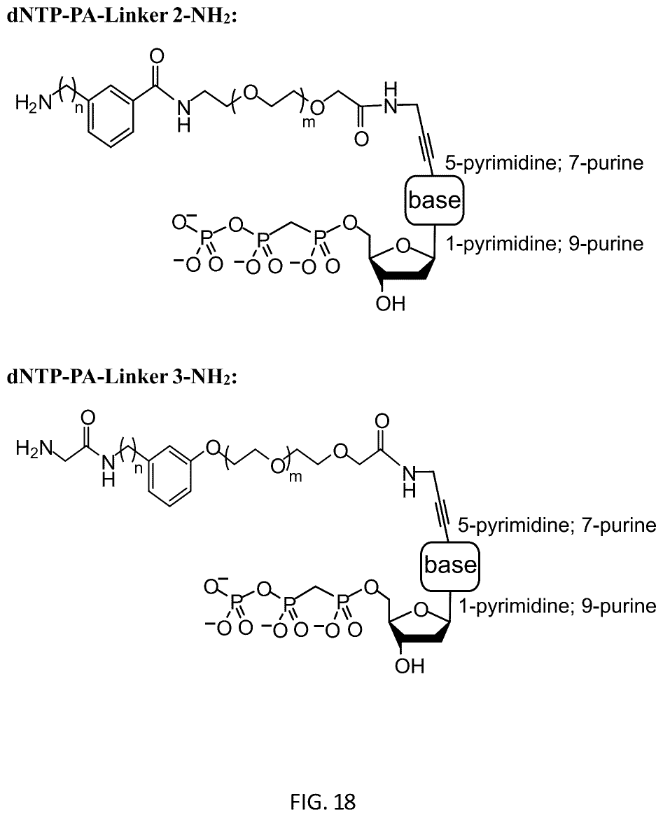

FIG. 18 shows the chemical structures of various exemplary linkers joined/attached to nucleotide units, according to some aspects.

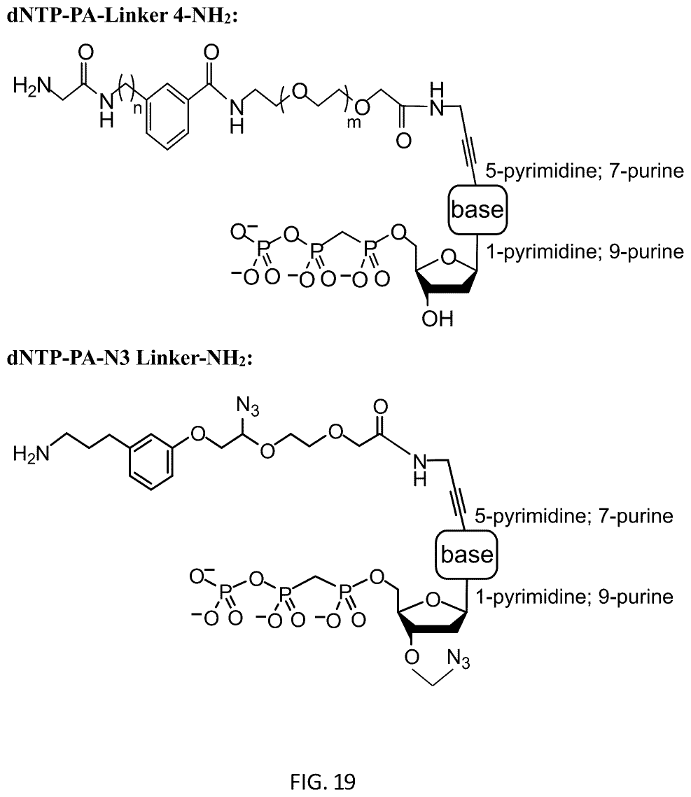

FIG. 19 shows the chemical structures of various exemplary linkers joined/attached to nucleotide units, according to some aspects.

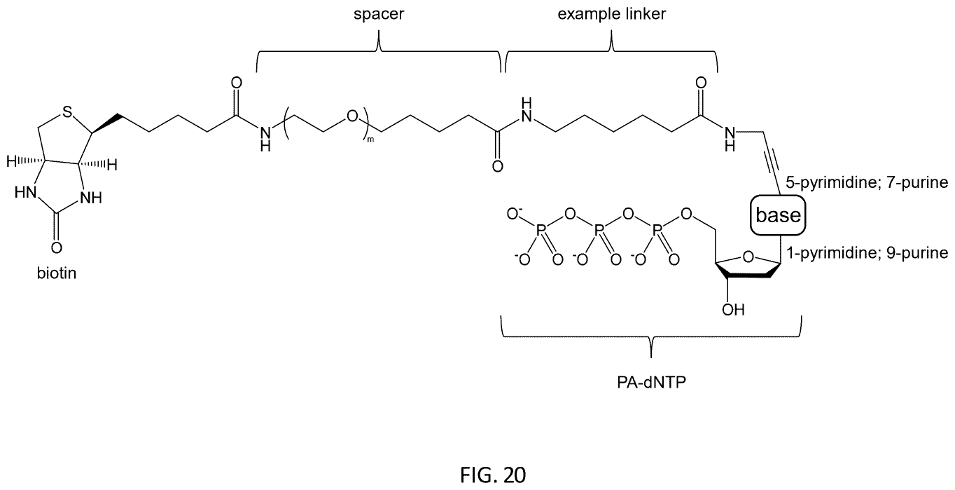

FIG. 20 shows the chemical structure of an exemplary biotinylated nucleotide-arm. In this example, the nucleotide unit is connected to the linker via a propargyl amine attachment at the 5 position of a pyrimidine base or the 7 position of a purine base, according to some aspects.

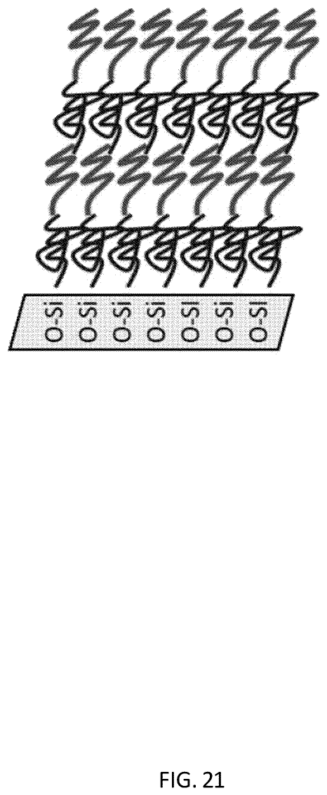

FIG. 21 shows a schematic illustration of one embodiment of the low binding solid supports of the present disclosure in which the support comprises a glass substrate and alternating layers of hydrophilic coatings which are covalently ornon-covalently adhered to the glass, and which further comprises chemically-reactive functional groups that serve as attachment sites for oligonucleotide primers.

In some aspects, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some aspects, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules may comprise one type multivalent molecule having one type of nucleotide unit selected from a group consisting of dATP, dGTP, dCTP, dTTP and dUTP. The plurality of multivalent molecules may comprise at a mixture of any combination of two or more types of multivalent molecules, where individual multivalent molecules in the mixture comprise nucleotide units selected from a group consisting of dATP, dGTP, dCTP, dTTP and/or dUTP.

In some aspects, the nucleotide unit comprises a chain of one, two or three phosphorus atoms where the chain is typically attached to the 5′ carbon of the sugar moiety via an ester or phosphoramide linkage. In some aspects, at least one nucleotide unit is a nucleotide analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

In some aspects, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some aspects, the nucleotide unit comprises a chain terminating moiety (e.g., blocking moiety) at the sugar 2′ position, at the sugar 3′ position, or at the sugar 2′ and 3′ position. In some aspects, the chain terminating moiety may inhibit polymerase-catalyzed incorporation of a subsequent nucleotide unit or free nucleotide in a nascent strand during a primer extension reaction. In some aspects, the chain terminating moiety is attached to the 3′ sugar position where the sugar comprises a ribose or deoxyribose sugar moiety. In some aspects, the chain terminating moiety is removable/cleavable from the 3′ sugar position to generate a nucleotide having a 3′OH sugar group which is extendible with a subsequent nucleotide in a polymerase-catalyzed nucleotide incorporation reaction. In some aspects, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some aspects, the chain terminating moiety is cleavable/removable from the nucleotide unit, for example by reacting the chain terminating moiety with a chemical agent, pH change, light or heat. In some aspects, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some aspects, the chain terminating moieties aryl and benzyl are cleavable with H2 Pd/C. In some aspects, the chain terminating moieties amine, amide, keto, isocyanate, phosphate, thio, disulfide are cleavable with phosphine or with a thiol group including beta-mercaptoethanol or dithiothritol (DTT). In some aspects, the chain terminating moiety carbonate is cleavable with potassium carbonate (K₂CO₃) in MeOH, with triethylamine in pyridine, or with Zn in acetic acid (AcOH). In some aspects, the chain terminating moieties urea and silyl are cleavable with tetrabutylammonium fluoride, pyridine-HF, with ammonium fluoride, or with triethylamine trihydrofluoride.

In some aspects, the nucleotide unit comprises a chain terminating moiety (e.g., blocking moiety) at the sugar 2′ position, at the sugar 3′ position, or at the sugar 2′ and 3′ position. In some aspects, the chain terminating moiety comprises an azide, azido or azidomethyl group. In some aspects, the chain terminating moiety comprises a 3′-O-azido or 3′-O-azidomethyl group. In some aspects, the chain terminating moieties azide, azido and azidomethyl group are cleavable/removable with a phosphine compound. In some aspects, the phosphine compound comprises a derivatized tri-alkyl phosphine moiety or a derivatized tri-aryl phosphine moiety. In some aspects, the phosphine compound comprises Tris(2-carboxyethyl)phosphine (TCEP) or bis-sulfo triphenyl phosphine (BS-TPP) or Tri(hydroxyproyl)phosphine (THPP). In some aspects, the cleaving agent comprises 4-dimethylaminopyridine (4-DMAP).

In some aspects, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some aspects, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some aspects, the detectable reporter moiety comprises a fluorophore. In some aspects, a particular detectable reporter moiety (e.g., fluorophore) that is attached to the multivalent molecule may correspond to the base (e.g., dATP, dGTP, dCTP, dTTP or dUTP) of the nucleotide unit to permit detection and identification of the nucleotide base.

In some aspects, at least one nucleotide arm of a multivalent molecule has a nucleotide unit that is attached to a detectable reporter moiety. In some aspects, the detectable reporter moiety is attached to the nucleotide base. In some aspects, the detectable reporter moiety comprises a fluorophore. In some aspects, a particular detectable reporter moiety (e.g., fluorophore) that is attached to the multivalent molecule may correspond to the base (e.g., dATP, dGTP, dCTP, dTTP or dUTP) of the nucleotide unit to permit detection and identification of the nucleotide base.

In some aspects, the core of a multivalent molecule comprises an avidin-like or streptavidin-like moiety and the core attachment moiety comprises biotin. In some aspects, the core comprises a streptavidin-type or avidin-type moiety which includes an avidin protein, as well as any derivatives, analogs and other non-native forms of avidin that may bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some aspects, any of the methods for sequencing nucleic acid molecules described herein may include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some aspects, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some aspects, the present disclosure provides said method wherein the binding complex is deposited on, attached to, or hybridized to, a surface showing a contrast to noise ratio in the detecting step of greater than 20. In some aspects, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

A compaction oligonucleotide comprises a single-stranded linear oligonucleotide having a 5′ region that may hybridize to a first portion of a concatemer molecule and the compaction oligonucleotide having a 3′ region that may hybridize to a second portion of the concatemer molecule (e.g., the same concatemer molecule). In some aspects, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball may be represented as a Gaussian spot and the size may be measured as a full width half maximum (FWHM). A smaller spot size as indicated by a smaller FWHM typically correlates with an improved image of the spot. In some aspects, the FWHM of a DNA nanoball spot may be about 10 um or smaller. The DNA nanoball may be a compact nucleic acid structure having a full width half maximum (FWHM) that is smaller compared to a concatemer that is not collapsed/folded into a DNA nanoball.

In some aspects, compaction oligonucleotides comprise a single stranded oligonucleotides comprising DNA, RNA, or a combination of DNA and RNA. The compaction oligonucleotides may be any length, including 20-150 nucleotides, or 30-100 nucleotides, or 40-80 nucleotides in length.

In some aspects, the compaction oligonucleotides comprises a 5′ region and a 3′ region, and optionally an intervening region between the 5′ and 3′ regions. The intervening region may be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides may be wholly complementary or partially complementary along its length to a first portion of a concatemer molecule. The 3′ region of the compaction oligonucleotides may be wholly complementary or partially complementary along its length to a second portion of a concatemer molecule. The 5′ region of the compaction oligonucleotides may hybridize to a first universal sequence portion of a concatemer molecule. The 3′ region of the compaction oligonucleotides may hybridize to a second universal sequence portion of a concatemer molecule. The 5′ and 3′ regions of the compaction oligonucleotide may hybridize to the concatemer to pull together distal portions of the concatemer causing compaction of the concatemer to form a DNA nanoball.

The 5′ region of the compaction oligonucleotide may have the same sequence as the 3′ region. The 5′ region of the compaction oligonucleotide may have a sequence that is different from the 3′ region. The 3′ region of the compaction oligonucleotide may have a sequence that is a reverse sequence of the 5′ region.

In some aspects sequence data may be derived through nanopore sequencing, which comprises sequencing of a nucleic acid by translocating said nucleic acid across a membrane, such as through a pore, and wherein sequence reads or base calls are made by measuring one or more signals during the translocation event, such as impedance, current, voltage, or capacitance. In some aspects, the identity of a nucleotide may be determined by distinctive electrical signatures, such as the timing, duration, extent, or lineshape of a current block, impedance change, voltage change, or capacitance change. Sequencing of nucleic acids by translocation across a membrane and/or through a pore does not foreclose alternative detection methods, such as optical, chemical, biochemical, fluorescent, luminescent, magnetic, electromagnetic, acoustic, or electroacoustic detection.

### Supports and Low Non-Specific Coatings

In some aspects, the flow cell 112 in FIG. 1 may include a support, e.g., a solid support as disclosed herein. Aspects of the present disclosure provide pairwise sequencing compositions and methods which employ a support comprising a plurality of oligonucleotide surface primers immobilized thereon. In some aspects, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers. In some aspects, the plurality of surface primers are immobilized to the low non-specific binding coating. In some aspects, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that may be used for tethering single-stranded nucleic acid library molecules to the support. In some aspects, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some aspects, the support structure may comprise one or more surfaces within an integrated or assembled microfluidic flow cell. The support structure may comprise one or more surfaces within a microplate format, e.g., the bottom surface of the wells in a microplate. In some aspects, the support structure comprises the interior surface (such as the lumen surface) of a capillary. In some aspects, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some aspects, the first layer may be covalently attached to the surface. In some aspects, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternatively or in combination, the coating may be patterned using, e.g., contact printing and/or ink-jet printing techniques. In some aspects, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some aspects, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface may include, but are not limited to, biotin, methoxy ether, carboxylate, amine, NHS ester, maleimide, and bis-silane. In some aspects, two or more layers of a hydrophilic polymer, e.g., a linear polymer, branched polymer, or multi-branched polymer, may be deposited on the surface. In some aspects, two or more layers may be covalently coupled to each other or internally cross-linked to improve the stability of the resulting coating. In some aspects, surface primers with different nucleotide sequences and/or base modifications (or other biomolecules, e.g., enzymes or antibodies) may be tethered to the resulting layer at various surface densities. In some aspects, for example, both surface functional group density and surface primer concentration may be varied to attain a desired surface primer density range. Additionally, surface primer density may be controlled by diluting the surface primers with other molecules that carry the same functional group. For example, amine-labeled surface primers may be diluted with amine-labeled polyethylene glycol in a reaction with an NHS-ester coated surface to reduce the final primer density. Surface primers with different lengths of linker between the hybridization region and the surface attachment functional group may also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some aspects, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some aspects, the functionalized polymer coating comprises a poly(N-(5-azidoacetamidylpentyl)acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” may be accomplished using traditional crosslinking approaches with any compatible polymer or monomer subunits such that a surface comprising two or more highly crosslinked layers may be built sequentially. Examples of suitable polymers include, but are not limited to, streptavidin, poly acrylamide, polyester, dextran, poly-lysine, and copolymers of poly-lysine and PEG. In some aspects, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some aspects, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some aspects, the surface of the support structure used for nucleic acid hybridization and amplification may be a solid, non-porous surface. In some aspects, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some aspects, the support structure comprises the interior surface (such as the lumen surface) of a capillary. In some aspects the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some aspects, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some aspects, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some aspects, the label may comprise a fluorescent label. In some aspects, the label may comprise a radioisotope. In some aspects, the label may comprise any other detectable label known to one of skill in the art. In some aspects, the degree of non-specific binding exhibited by a given support surface formulation may thus be assessed in terms of the number of non-specifically bound protein molecules (or nucleic acid molecules or other molecules) per unit area. In some aspects, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per m², less than 0.01 molecule per m², less than 0.1 molecule per m², less than 0.25 molecule per m², less than 0.5 molecule per m², less than 1 molecule per m², less than 10 molecules per m², less than 100 molecules per m², or less than 1,000 molecules per m². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 m. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beam splitters, and band pass filters were chosen as 532 LP or 645 LP concordant with the appropriate excitation wavelength. Some modified surfaces disclosed herein exhibit nonspecific binding of dye molecules of less than 0.25 molecules per m². In some aspects, the coated support was immersed in a buffer (e.g., 25 mM ACES, pH 7.4) while the image was acquired.

In some aspects, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some aspects, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some aspects, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some aspects, a static contact angle may be determined. In some aspects, an advancing or receding contact angle may be determined. In some aspects, the water contact angle for the hydrophilic, low-binding support surface disclosed herein may range from about 0 degrees to about 30 degrees. In some aspects, the water contact angle for the hydrophilic, low-binding support surface disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some aspects, the hydrophilic surfaces disclosed herein facilitate reduced wash times for bioassays, often due to reduced nonspecific binding of biomolecules to the low-binding surfaces. In some aspects, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some aspects, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some aspects, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some aspects, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some aspects, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some aspects, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some aspects, 1 primer or adapter sequence may be tethered to at least one layer of the surface. In some aspects, at least 2, 3, 4, 5, 6, 7, 8, 9, 10, or more than 10 different primer or adapter sequences may be tethered to at least one layer of the surface.

In some aspects, the tethered adapter and/or primer sequences may range in length from about 10 nucleotides to about 100 nucleotides. In some aspects, the tethered adapter and/or primer sequences may be at least 10, at least 20, at least 30, at least 40, at least 50, at least 60, at least 70, at least 80, at least 90, or at least 100 nucleotides in length. In some aspects, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some aspects the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some aspects, the resultant surface density of primers (e.g., capture primers) on the low binding support surfaces of the present disclosure may range from about 100 primer molecules per μm² to about 100,000 primer molecules per μm². In some aspects, the resultant surface density of primers on the low binding support surfaces of the present disclosure may range from about 1,000 primer molecules per μm² to about 1,000,000 primer molecules per m². In some aspects, the surface density of primers may be at least 1,000, at least 10,000, at least 100,000, or at least 1,000,000 molecules per m². In some aspects, the surface density of primers may be at most 1,000,000, at most 100,000, at most 10,000, or at most 1,000 molecules per μm². Any of the lower and upper values described in this paragraph may be combined to form a range included within the present disclosure, for example, in some aspects the surface density of primers may range from about 10,000 molecules per μm² to about 100,000 molecules per μm². Those of skill in the art will recognize that the surface density of primer molecules may have any value within this range, e.g., about 455,000 molecules per m². In some aspects, the surface density of target library nucleic acid sequences initially hybridized to adapter or primer sequences on the support surface may be less than or equal to that indicated for the surface density of tethered primers. In some aspects, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some aspects, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it may be shown that improved CNR may provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) may be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical NGS applications, this background signal in the current field-of-view (FOV) is averaged over time and subtracted. The signal arising from individual DNA colonies (i.e., (Signal)-B(interstial) in the FOV) yields a discernable feature that may be classified. In some aspects, the intrastitial background (B(intrastitial)) may contribute a confounding fluorescence signal that is not specific to the target of interest, but is present in the same ROI thus making it far more difficult to average and subtract.

Nucleic acid amplification on the low-binding coated supports described herein may decrease the B(interstitial) background signal by reducing non-specific binding, may lead to improvements in specific nucleic acid amplification, and may lead to a decrease in non-specific amplification that may impact the background signal arising from both the interstitial and intrastitial regions. In some aspects, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The headings provided herein are not limitations of the various aspects of the disclosure, which aspects may be understood by reference to the specification as awhole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken to mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that may be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” may mean within one or more than one standard deviation per the practice in the art. Alternatively, “about” or “approximately” may mean a range of up to 10% (i.e., ±10%) or more depending on the limitations of the measurement system. For example, about 5 mg may include any number between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges may include the endpoints of the ranges and/or subranges.

The term “polony” used herein refers to a nucleic acid library molecule that may be clonally amplified in-solution or on-support to generate an amplicon that may serve as a template molecule for sequencing. In some aspects, a linear library molecule may be circularized to generate a circularized library molecule, and the circularized library molecule may be clonally amplified in-solution or on-support to generate a concatemer. In some aspects, the concatemer may serve as a nucleic acid template molecule which may be sequenced. The concatemer is sometimes referred to as a polony. In some aspects, a polony includes denatured, cloned nucleotide strands. In some embodiments, the methods disclosed herein are configured to work with flow cell images containing polonies or their similar signal spots, e.g., clusters of signals.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that may catalyze polymerization of nucleotides (including analogs thereof) into a nucleic acid strand. Typically but not necessarily such nucleotide polymerization may occur in a template-dependent fashion. Typically, a polymerase comprises one or more active sites at which nucleotide binding and/or catalysis of nucleotide polymerization may occur. In some aspects, a polymerase includes other enzymatic activities, such as for example, 3′ to 5′ exonuclease activity or 5′ to 3′ exonuclease activity. In some aspects, a polymerase has strand displacing activity. A polymerase may include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some aspects, a polymerase may be isolated from a cell, or generated using recombinant DNA technology or chemical synthesis methods. In some aspects, a polymerase may be expressed in prokaryote, eukaryote, viral, or phage organisms. In some aspects, a polymerase may be post-translationally modified proteins or fragments thereof. A polymerase may be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and the 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide may be complementary or non-complementary to a nucleotide residue in the template molecule. The nucleotide unit or the free nucleotide may bind to the 3′ end of the nucleic acid primer at a position that is opposite a complementary nucleotide residue in the nucleic acid template molecule. The persistence time is indicative of the stability of the binding complex and strength of the binding interactions. Persistence time may be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids may be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some aspects, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer may include a 3′ OH moiety which serves as a nucleotide polymerization initiation site in a polymerase-mediated primer extension reaction. Alternatively, the 3′ end of the primer may lack a 3′ OH moiety, or may include a terminal 3′ blocking group that inhibits nucleotide polymerization in a polymerase-mediated reaction. Any one nucleotide, or more than one nucleotide, along the length of the primer may be labeled with a detectable reporter moiety. A primer may be in solution (e.g., a soluble primer) or may be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid may be single-stranded or double-stranded, or the template nucleic acid may have single-stranded or double-stranded portions. The sequence of the template nucleic acid may be partially or wholly complementary to the sequence of the complementary strand. The template nucleic acid may be obtained from a naturally-occurring source, recombinant form, or chemically synthesized to include any type of nucleic acid analog. The template nucleic acid may be linear, circular, or other forms. The template nucleic acids may include an insert region having an insert sequence which is also known as a sequence of interest. The template nucleic acids may also include at least one adaptor sequence. The template nucleic acid may be a concatemer having two or tandem copies of a sequence of interest and at least one adaptor sequence. The insert region may be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region may be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region may be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid may be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization may comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing may be the standard A-T or C-G base pairing, or may be other forms of base-pairing interactions. Duplex nucleic acids may include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some aspects comprises a monophosphate, diphosphate, or triphosphate, or corresponding phosphate analog. In some aspects, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases may be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some aspects, nucleotides comprise a chain of one, two or three phosphorus atoms where the chain is typically attached to the 5′ carbon of the sugar moiety via an ester or phosphoramide linkage. In some aspects, the nucleotide is an analog having a phosphorus chain in which the phosphorus atoms are linked together with intervening O, S, NH, methylene or ethylene. In some aspects, the phosphorus atoms in the chain include substituted side groups including O, S or BH₃. In some aspects, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation may be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties may be selected having spectrally distinct emission profiles, or having minimal overlapping spectral emission profiles. Reporter moieties may be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some aspects, the reporter moiety may be a FRET pair, such that multiple classifications may be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure may include but are not limited to: nucleotide transient-binding; nucleotide incorporation; de-blocking; washing; removing; flowing; detecting; imaging and/or identifying. Such linkage may comprise, for example, covalent, ionic, hydrogen, dipole-dipole, hydrophilic, hydrophobic, or affinity bonding, bonds or associations involving van der Waals forces, mechanical bonding, and the like. In some aspects, such linkage occurs intramolecularly, for example linking together the ends of a single-stranded or double-stranded linear nucleic acid molecule to form a circular molecule. In some aspects, such linkage may occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages may be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components may be linked together covalently. For example, two nucleic acid components may be enzymatically ligated together where the linkage that joins together the two components comprises phosphodiester linkage. A first and second nucleic acid component may be linked together, where the first nucleic acid component may confer a function on a second nucleic acid component. For example, linkage between a primer binding sequence and a sequence of interest forms a nucleic acid library molecule having a portion that may bind to a primer. In another example, a transgene (e.g., a nucleic acid encoding a polypeptide or a nucleic acid sequence of interest) may be ligated to a vector where the linkage permits expression or functioning of the transgene sequence contained in the vector. In some aspects, a transgene is operably linked to a host cell regulatory sequence (e.g., a promoter sequence) that affects expression of the transgene. In some aspects, the vector comprises at least one host cell regulatory sequence, including a promoter sequence, enhancer, transcription and/or translation initiation sequence, transcription and/or translation termination sequence, polypeptide secretion signal sequences, and the like. In some aspects, the host cell regulatory sequence controls expression of the level, timing and/or location of the transgene.

The term “adaptor” and related terms refers to oligonucleotides that may be operably linked (appended) to a target polynucleotide, where the adaptor confers a function to the co-joined adaptor-target molecule. Adaptors comprise DNA, RNA, chimeric DNA/RNA, or analogs thereof. Adaptors may include at least one ribonucleoside residue. Adaptors may be single-stranded, double-stranded, or have single-stranded and/or double-stranded portions. Adaptors may be configured to be linear, stem-looped, hairpin, or Y-shaped forms. Adaptors may be any length, including 4-100 nucleotides or longer. Adaptors may have blunt ends, overhang ends, or a combination of both. Overhang ends include 5′ overhang and 3′ overhang ends. The 5′ end of a single-stranded adaptor, or one strand of a double-stranded adaptor, may have a 5′ phosphate group or lack a 5′ phosphate group. Adaptors may include a 5′ tail that does not hybridize to a target polynucleotide (e.g., tailed adaptor), or adaptors may be non-tailed. An adaptor may include a sequence that is complementary to at least a portion of a primer, such as an amplification primer, a sequencing primer, or a capture primer (e.g., soluble or immobilized capture primers). Adaptors may include a random sequence or degenerate sequence. Adaptors may include at least one inosine residue. Adaptors may include at least one phosphorothioate, phosphorothiolate and/or phosphoramidate linkage. Adaptors may include a barcode sequence which may be used to distinguish polynucleotides (e.g., insert sequences) from different sample sources in a multiplex assay. Adaptors may include a unique identification sequence (e.g., unique molecular index, UMI; or a unique molecular tag) that may be used to uniquely identify a nucleic acid molecule to which the adaptor is appended. In some aspects, a unique identification sequence may be used to increase error correction and accuracy, reduce the rate of false-positive variant calls and/or increase sensitivity of variant detection. Adaptors may include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence may be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

In some aspects, the support is solid, semi-solid, or a combination of both. In some aspects, the support is porous, semi-porous, non-porous, or any combination of porosity. In some aspects, the support may be substantially planar, concave, convex, or any combination thereof. In some aspects, the support may be cylindrical, for example comprising a capillary or interior surface of a capillary.

In some aspects, the surface of the support may be substantially smooth. In some aspects, the support may be regularly or irregularly textured, including bumps, etched, pores, three-dimensional scaffolds, or any combination thereof.

In some aspects, the support comprises a bead having any shape, including spherical, hemi-spherical, cylindrical, barrel-shaped, toroidal, disc-shaped, rod-like, conical, triangular, cubical, polygonal, tubular or wire-like.

The support may be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some aspects, the surface of the support is coated with one or more compounds to produce a passivated layer on the support. In some aspects, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some aspects, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some aspects, a static contact angle may be determined. In some aspects, an advancing or receding contact angle may be determined. In some aspects, the water contact angle for the hydrophilic, low-binding support surface disclosed herein may range from about 0 degrees to about 30 degrees. In some aspects, the water contact angle for the hydrophilic, low-binding support surface disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases thecontact angle is no more than 40 degrees. Those of skill in the art will realize that a given hydrophilic, low-binding support surface of the present disclosure may exhibit a water contact angle having a value of anywhere within this range.

The present disclosure provides a plurality (e.g., two or more) of nucleic acid templates immobilized to a support. In some aspects, the immobilized plurality of nucleic acid templates have the same sequence or have different sequences. In some aspects, individual nucleic acid template molecules in the plurality of nucleic acid templates are immobilized to a different site on the support. In some aspects, two or more individual nucleic acid template molecules in the plurality of nucleic acid templates are immobilized to a site on the support. In some aspects, the support comprises a plurality of sites arranged in an array. The term “array” refers to a support comprising a plurality of sites located at pre-determined locations on the support to form an array of sites. The sites may be discrete and separated by interstitial regions. In some aspects, the pre-determined sites on the support may be arranged in one dimension in a row or a column, or arranged in two dimensions in rows and columns. In some aspects, the plurality of pre-determined sites is arranged on the support in an organized fashion. In some aspects, the plurality of pre-determined sites is arranged in any organized pattern, including rectilinear, hexagonal patterns, grid patterns, patterns having reflective symmetry, patterns having rotational symmetry, or the like. The pitch between different pairs of sites may be that same or may vary. In some aspects, the support may have nucleic acid template molecules immobilized at a plurality of sites at a surface density of about 10²-10¹⁵ sites per mm², or more, to form a nucleic acid template array. In some aspects, the support comprises at least 10² sites, at least 10³ sites, at least 10⁴ sites, at least 10⁵ sites, at least 10⁶ sites, at least 10⁷ sites, at least 10⁸ sites, at least 109 sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some aspects, a plurality of pre-determined sites on the support (e.g., 10²-10¹⁵ sites or more) are immobilized with nucleic acid templates to form a nucleic acid template array. In some aspects, the nucleic acid templates that are immobilized at a plurality of pre-determined sites by hybridization to immobilized surface capture primers, or the nucleic acid templates are covalently attached to the surface capture primers. In some aspects, the nucleic acid templates are immobilized at a plurality of pre-determined sites, for example, at 10²-10¹⁵ sites or more. In some aspects, the nucleic acid templates that are immobilized at a plurality of sites on the support comprise linear or circular nucleic acid template molecules or a mixture of both linear and circular molecules. In some aspects, the immobilized nucleic acid templates are clonally-amplified to generate immobilized nucleic acid polonies at the plurality of pre-determined sites. In some aspects, individual immobilized nucleic acid template molecules comprise one copy of a target sequence of interest, or comprise concatemers having two or more tandem copies of a target sequence of interest.

In some aspects, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some aspects, the support comprises at least 102 sites, at least 10³ sites, at least 10⁴ sites, at least 10⁵ sites, at least 10⁶ sites, at least 10⁷ sites, at least 10⁸ sites, at least 109 sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some aspects, a plurality of randomly located sites on the support (e.g., 10²-10¹⁵ sites or more) are immobilized with nucleic acid templates to form a support immobilized with nucleic acid templates. In some aspects, the nucleic acid templates that are immobilized at a plurality of randomly located sites by hybridization to immobilized surface capture primers, or the nucleic acid templates are covalently attached to the surface capture primer. In some aspects, the nucleic acid templates are immobilized at a plurality of randomly located sites, for example, at 10²-10¹⁵ sites or more. In some aspects, the nucleic acid templates that are immobilized at a plurality of sites on the support comprise linear or circular nucleic acid template molecules or a mixture of both linear and circular molecules. In some aspects, the immobilized nucleic acid templates are clonally-amplified to generate immobilized nucleic acid polonies at the plurality of randomly located sites. In some aspects, individual immobilized nucleic acid template molecules comprise one copy of a target sequence of interest, or comprise concatemers having two or more tandem copies of a target sequence of interest.

In some aspects, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support may be reacted with the reagents in a massively parallel manner. In some aspects, the fluid communication of the plurality of immobilized nucleic acid template molecules may be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some aspects, the term “immobilized” and related terms refer to nucleic acid molecules or enzymes (e.g., polymerases) that are attached to the support at pre-determined or random locations, where the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some aspects, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some aspects, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some aspects, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some aspects, the number of layers is at least 1, at least 2, at least 3, at least 4, at least 5, at least 6, at least 7, at least 8, at least 9, or at least 10. In some aspects, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some aspects the number of layers may range from about 2 to about 4. In some aspects, all of the layers may comprise the same material. In some aspects, each layer may comprise a different material. In some aspects, the plurality of layers may comprise a plurality of materials. In some aspects at least one layer may comprise a branched polymer. In some aspects, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some aspects the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some aspects, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some aspects, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group may be either on the side chain of the polymer or directly attaches to a central core or central backbone of the polymer. The branched polymer may have linear backbone with one or more functional groups coming off the backbone for conjugation. The branched polymer may also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and its variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adaptor sequence. In some aspects, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, typically by determining the identity of at least some nucleotides (including their nucleobase components) within the nucleic acid template molecule. While in some aspects, “sequencing” a given region of a nucleic acid molecule includes identifying each and every nucleotide within the region that is sequenced, in some aspects “sequencing” comprises methods whereby the identity of only some of the nucleotides in the region is determined, while the identity of some nucleotides remains undetermined or incorrectly determined. Any suitable method of sequencing may be used. In an exemplary aspect, sequencing may include label-free or ion based sequencing methods. In some aspects, sequencing may include labeled or dye-containing nucleotide or fluorescent based nucleotide sequencing methods. In some aspects, sequencing may include polony-based sequencing or bridge sequencing methods. In some aspects, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299(5607):682-686; Eid, et al., 2009 Science 323(5910): 133-138; U.S. Pat. Nos. 7,170,050; 7,302,146; and 7,405,281). An example of sequence-by-hybridization includes SOLiD sequencing (e.g., from Life Technologies; WO 2006/084132). An example of sequence-by-binding includes Omniome sequencing (e.g., U.S. Pat. No. 10,246,744).

It is to be appreciated that the Detailed Description section, and not any other section, is intended to be used to interpret the claims. Other sections may set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative aspects may perform functional blocks, steps, operations, methods, etc. using orderings different from those described herein.

References herein to “one aspect,” “an aspect,” “an example aspect,” “some aspects,” or similar phrases, indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein.

Additionally, some aspects may be described using the expression “coupled” and “connected” along with their derivatives. These terms are not necessarily intended as synonyms for each other. For example, some aspects may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

### In Situ Sequencing

In the methods 500, 600, 2300 described herein, the flow cell images can be acquired or generated from 2D or 3D samples. In the embodiments with 3D cellular sample(s), the RNA is not extracted from the cellular sample and sequencing information does not need to be tracked and mapped back to an image of the cellular sample. Rather, RNA may be retained inside the cellular sample to permit direct imaging of the spatial location of target RNAs within the cells. Additionally, RNA within the cellular sample may not be fragmented and enrichment of target RNA is not necessary. Use of target-specific and/or random-sequence reverse transcription primers enables detection of both poly-A and non-poly-A RNAs in either uni-plex or multi-plex modes.

In some embodiments, the methods 500, 600, 2300 comprise repeatedly conducting a short number of sequencing cycles of the same region of the template molecules (e.g., concatemer molecules). By conducting reiterative short sequencing cycles, the RNA content of the cellular sample can be discovered. Compared to long read sequencing workflows, the reiterative short sequencing cycles described herein use a reduced amount of sequencing reagents which reduces cost and saves time. Methods for conducting reiterative short sequencing cycles has many uses including but not limited to detecting specific RNAs of interest, mutant RNA sequences, splice variants, and their abundance levels thereof.

The concatemers carry tandem repeat units of a cDNA-of-interest, the universal sequencing primer binding site, and the target barcode sequence. The concatemers are sequenced inside the cellular sample where a short number of sequencing cycles are conducted for each round and multiple rounds of short read sequencing is conducted. The full length of the target barcode and cDNA region are not sequenced. Instead, at least a portion of the target barcode region is reiteratively sequenced. In some embodiments, it is not necessary to sequence the cDNA region. In some embodiments, the target barcode and a portion of the cDNA region are reiteratively sequenced. It is not necessary to sequence the entire length of the cDNA region. It is not necessary to assemble the sequencing reads or to obtain a full length sequence of the cDNAs-of-interest. The redundant sequencing information obtained from the short sequencing reads obviates the need to sequence the complementary strand of the concatemer. Thus pairwise sequencing is not necessary.

Additionally, a short portion of the cDNA region in the concatemer is re-sequenced at least once (e.g., reiterative sequencing) from the same start position to generate overlapping sequencing reads that can be aligned to a reference sequence. For example, the same portion of the concatemer molecule can be sequenced at least two, three, four, five, or up to 50 times. The start sequencing site can be any location of the concatemer and is dictated by the sequencing primers which are designed to anneal to a selected position within the concatemer. The reiterative short sequencing reads increase the redundancy of sequencing information for individual bases in the cDNA region. Reiteratively sequencing one strand of the concatemer template molecule provides enough base coverage to reveal the presence of target RNAs in the cellular sample so that pairwise sequencing of the complementary strand is not necessary.

A concatemer template molecule includes multiple sequencing primer binding sites along the same concatemer molecule which can be used to generate multiple usable sequencing reads for increased sequencing depth. Together, reiteratively sequencing one strand of the concatemer templates increases sequencing base coverage and sequencing depth compared to sequencing a one-copy template molecule.

The methods of conducting sequencing reactions described herein can be conducted in uni-plex or multi-plex modes. Two or more different target RNAs can be detected and imaged simultaneously inside a cellular sample using different reverse transcription primers, different target-specific padlock probes, and universal sequencing primers. For example, the presence of a housekeeping RNA and at least one target RNA in a cellular sample can be simultaneously detected and imaged using any of the reiterative short read sequencing methods described herein.

The present disclosure provides methods for conducting sequencing reactions that detects in situ at least two different target RNA molecules in a cellular sample comprising step (a): providing a cellular sample harboring a plurality of RNA which comprises at least a first target RNA molecule and a second target RNA molecule. In some embodiments, the cellular sample is fixed and permeabilized. In some embodiments, the cellular sample harbors 2-25 different target RNA molecules, or harbors 25-50 different target RNA molecules, or harbors 50-75 different target RNA molecules, or harbors 75-100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, an FFPE cellular sample, or a sectioned FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the cellular sample is cultured before or after depositing the cellular sample onto the solid support. In some embodiments, the cellular sample is cultured prior to conducting step (b) which is described below. In some embodiments, the cellular sample comprises an expanded cellular sample that has been cultured in a simple or complex cell culture media. In some embodiments, the cellular sample is not cultured or expanded prior to conducting step (b).

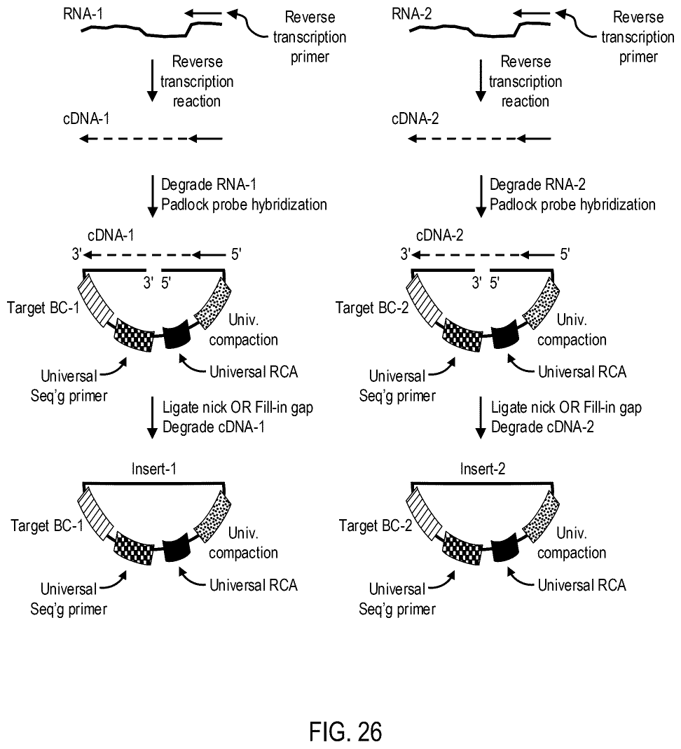

In some embodiments, methods for conducting sequencing reactions that detects in situ at least two different target RNA molecules in a cellular sample further comprise step (b): generating inside the cellular sample a plurality of cDNA molecules which include at least a first target cDNA molecule that corresponds to the first target RNA molecule, and the plurality of cDNA molecules includes a second target cDNA molecule that corresponds to the second target RNA molecule. In some embodiments, the method comprises generating at least 2-10,000 different target cDNA molecules that correspond to 2-10,000 different target RNA molecules. In some embodiments, the generating of step (b) comprises contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample (e.g., FIG. 26).

In some embodiments, the plurality of reverse transcription primers comprises a first sub-population of target-specific reverse transcription primers that hybridize selectively to the first target RNA, and comprises a second sub-population of target-specific reverse transcription primers that hybridize selectively to the second target RNA. In some embodiments, the first and second sub-population of target-specific reverse transcription primers have the same sequence or different sequences.

In some embodiments, the entire length of the first sub-population of target-specific reverse transcription primers hybridize to a first target RNA molecule. In some embodiments, the first sub-population of target-specific reverse transcription primers comprise tailed primers having a portion that hybridizes to a first target RNA molecule and a portion that does not hybridize to a first target RNA molecule. In some embodiments, the first sub-population of target-specific reverse transcription primers comprise at least a portion having a poly-T sequence. In some embodiments, the first sub-population of target-specific reverse transcription primers comprise at least a portion having a random sequence and/or at least a portion having a target-specific sequence.

In some embodiments, the entire length of the second sub-population of target-specific reverse transcription primers hybridize to a second target RNA molecule. In some embodiments, the second sub-population of target-specific reverse transcription primers comprise tailed primers having a portion that hybridizes to a second target RNA molecule and a portion that does not hybridize to a second target RNA molecule. In some embodiments, the second sub-population of target-specific reverse transcription primers comprise at least a portion having a poly-T sequence. In some embodiments, the second sub-population of target-specific reverse transcription primers comprise at least a portion having a random sequence and/or at least a portion having a target-specific sequence.

In some embodiments, a target RNA molecule that is hybridized to a cDNA molecule can be subjected to enzymatic degradation using a ribonuclease under a condition suitable for degrading RNA in an RNA/DNA duplex. In some embodiments, a target RNA molecule that is hybridized to a cDNA molecule is not subjected to enzymatic degradation.

In some embodiments, methods for conducting sequencing reactions that detects in situ at least two different target RNA molecules in a cellular sample further comprise step (c): contacting the plurality of cDNA molecules in the cellular sample with a plurality of target-specific padlock probes which includes at least a first plurality of target-specific padlock probes and a second plurality of target-specific padlock probes. In some embodiments, the method comprises contacting the plurality of cDNA molecule in the cellular sample with at least 2-10,000 different target-specific padlock probes.

In an alternative embodiment, cDNA is not generated from RNA inside the cellular sample. In some embodiments, methods for detecting at least two different target RNA molecules in a cellular sample further comprise contacting RNA inside the cell with a plurality of target-specific padlock probes and generating circularized padlock probes. In some embodiments, methods for detecting at least two different target RNA molecules in a cellular sample further comprise step (c): contacting the plurality of RNA molecules in the cellular sample with a plurality of target-specific padlock probes which includes at least a first plurality of target-specific padlock probes and a second plurality of target-specific padlock probes. In some embodiments, the method comprises contacting the plurality of cDNA molecule in the cellular sample with at least 2-10,000 different target-specific padlock probes. In some embodiments, a target RNA molecule can be subjected to enzymatic degradation using a ribonuclease. In some embodiments, a target RNA molecule is not subjected to enzymatic degradation.

In some embodiments, individual padlock probes in the plurality of first target-specific padlock probes comprise first and second terminal regions (e.g., first and second padlock binding arms), wherein the first terminal region selectively hybridizes to a first region of the first target cDNA molecule (or the first target RNA molecule), and the second terminal region selectively hybridizes to a second region of the first target cDNA molecule (or the first target RNA molecule). In some embodiments, the contacting of step (c) comprises: hybridizing the first and second terminal regions of the first target-specific padlock probes to proximal positions on the first target cDNA molecule (or the first target RNA molecule) to form a circularized first target-specific padlock probe having a nick or gap between the hybridized first and second terminal regions (e.g., FIG. 26, left). In some embodiments, the first target-specific padlock probe comprises a first target barcode sequence (target BC-1) that corresponds to and uniquely identifies the first target cDNA sequence (or the first target RNA sequence). In some embodiments, the first target-specific padlock probe comprises a first target barcode sequence that is located adjacent to one of the regions of the first target-specific padlock probe that selectively hybridizes to the first target cDNA molecule (or the first target RNA sequence). In some embodiments, the first target-specific padlock probe comprises at least one universal adaptor sequence, such as for example a universal sequencing primer binding site (or a complementary sequence thereof). In some embodiments, the first target-specific padlock probe comprises a universal primer binding site for a rolling circle amplification primer (or a complementary sequence thereof). In some embodiments, the first target-specific padlock probe comprises a universal compaction oligonucleotide binding site (or a complementary sequence thereof).

FIG. 26 is a schematic showing a workflow for generating inside a cell circularized padlock probes, comprising generating first and second cDNAs from first and second target RNA molecules (respectively), hybridizing first and second padlock probes to the first and second cDNA molecules (respectively) to generate first and second circularized padlock probes (respectively). The first padlock probe comprises (i) a first target barcode sequence (target BC-1) that uniquely identifies the first target RNA or the first target cDNA, (ii) a first sequencing primer binding site (or a complementary sequence thereof), (iii) a universal binding site for an amplification primer (universal RCA) (or a complementary sequence thereof), and (iv) a universal binding site for a compaction oligonucleotide (or a complementary sequence thereof). The second padlock probe comprises (i) a second target barcode sequence (target BC-2) that uniquely identifies the second target RNA or the second target cDNA, (ii) a second sequencing primer binding site(or a complementary sequence thereof), (iii) a universal binding site for an amplification primer (universal RCA) (or a complementary sequence thereof), and (iv) a universal binding site for a compaction oligonucleotide (or a complementary sequence thereof).

In some embodiments, individual padlock probes in the plurality of second target-specific padlock probes comprise first and second terminal regions (e.g., first and second padlock binding arms), wherein the first terminal region selectively hybridizes to a first region of the second target cDNA molecule (or the second target RNA molecule), and the second terminal region selectively hybridizes to a second region of the second target cDNA molecule (or the second target RNA molecule). In some embodiments, the contacting of step (c) comprises: hybridizing the first and second terminal regions of the second target-specific padlock probes to proximal positions on the second target cDNA molecule (or the second target RNA molecule) to form a circularized second target-specific padlock probe having a nick or gap between the hybridized first and second terminal regions (e.g., FIG. 26, right). In some embodiments, the second target-specific padlock probe comprises a second target barcode sequence (target BC-2) that corresponds to and uniquely identifies the second target cDNA sequence (or the second target RNA sequence). In some embodiments, the second target-specific padlock probe comprises a second target barcode sequence that is located adjacent to one of the regions of the second target-specific padlock probe that selectively hybridizes to the second target cDNA molecule (or the second target RNA sequence). In some embodiments, the second target-specific padlock probe comprises at least one universal adaptor sequence, such as for example a universal sequencing primer binding site (or a complementary sequence thereof). In some embodiments, the second target-specific padlock probe comprises a universal primer binding site for a rolling circle amplification primer (or a complementary sequence thereof). In some embodiments, the second target-specific padlock probe comprises a universal compaction oligonucleotide binding site (or a complementary sequence thereof).

In some embodiments, the first target barcode sequence (target BC-1) and the second target barcode sequence (target BC-2) have different sequences and can be used to conduct multiplex RNA detection and sequencing. In some embodiments, the first target barcode sequence (target BC-1) and the second target barcode sequence (target BC-2) have the same sequence and can be used to conduct uni-plex RNA detection and sequencing.

In some embodiments, the first and second target-specific padlock probes comprise a universal sequencing primer binding site and a target barcode sequence that are adjacent to each other so that the target barcode region of the concatemer is sequenced first. The target barcode sequence can be any length, for example 3-15 bases, or 15-25 bases, or 25-40 bases, or longer.

In some embodiments, methods for conducting sequencing reactions that detects in situ at least two different target RNA molecules in a cellular sample further comprising step (d): closing the nick or gap in the at least first and second circularized target-specific padlock probes by conducting an enzymatic reaction, thereby generating at least a first covalently closed circular padlock probe and a second covalently closed circular padlock probe inside the cellular sample. In some embodiments, the closing the nick in the first and second circularized padlock probes comprises conducting an enzymatic ligation reaction. In some embodiments, closing the gap in the first and second circularized padlock probes comprises conducting a polymerase-catalyzed fill-in reaction using the first or second target cDNA molecule (or the first or second RNA molecule) as a template, and conducting an enzymatic ligation reaction. In some embodiments, the method comprises closing the nick or gap in at least 2-10,000 circularized target-specific padlock probes by conducting one or more enzymatic reactions, thereby generating at least 2-10,000 covalently closed circular padlock probes inside the cellular sample.

In some embodiments, methods for conducting sequencing reactions that detects in situ at least two different target RNA molecules in a cellular sample further comprising step (e): conducting a rolling circle amplification reaction inside the cellular sample using the first and second covalently closed circular padlock probes as template molecules, thereby generating a plurality of concatemer molecules including at least a first concatemer molecule that corresponds to a first target RNA molecule, and the plurality of concatemer molecules includes at least a second concatemer molecule that corresponds to a second target RNA molecule. In some embodiments, the first concatemer molecule comprises tandem repeat units, wherein a unit comprises a sequence that corresponds to the first target cDNA (or the first target RNA), the first target barcode sequence, and the universal sequencing primer binding site (or a complementary sequence thereof). In some embodiments, the second concatemer molecule comprises tandem repeat units, wherein a unit comprises a sequence that corresponds to the second target cDNA (or the second target RNA), the second target barcode sequence, and the universal sequencing primer binding site (or a complementary sequence thereof).

In some embodiments, the rolling circle amplification reaction of step (e) comprises contacting the covalently closed circularized padlock probes with an amplification primer (e.g., a universal rolling circle amplification primer), a strand-displacing DNA polymerase, and a plurality of nucleotides, under a condition suitable for hybridizing individual amplification primers to a covalently closed padlock probe, and under a condition suitable for conducting primer extension using the covalently closed padlock probe as a template molecule to generate a nucleic acid concatemer. In some embodiments, the method comprises conducting a rolling circle amplification reaction inside the cellular sample using the at least 2-10,000 covalently closed circular padlock probes as template molecules, thereby generating at least 2-10,000 concatemer molecules that correspond to at least 2-10,000 target RNA molecules. In some embodiments, the plurality of concatemers that are generated inside the cellular sample collapse into a DNA nanoball having a shape and size that is more compact compared to a non-collapsed concatemer.

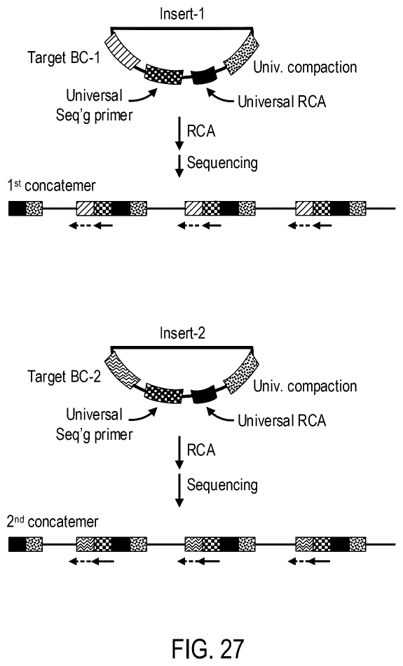

In some embodiments, methods for conducting sequencing reactions that detects in situ at least two different target RNA molecules in a cellular sample further comprising step (f): sequencing the plurality of concatemer molecules inside the cellular sample, which comprises sequencing the first concatemer molecule by conducting no more than 2-30 sequencing cycles to generate a plurality of first sequencing read products, and sequencing the second concatemer molecule by conducting no more than 2-30 sequencing cycles to generate a plurality of second sequencing read products (FIG. 27). In some embodiments, the sequencing of step (f) comprises sequencing no more than 2-30 bases of the first concatemer molecules to generate a plurality of first sequencing read products, and which comprises sequencing no more than 2-30 bases of the second concatemer molecules to generate a plurality of second sequencing read products. In some embodiments, the method comprises sequencing the at least 2-10,000 concatemer molecules inside the cellular sample, which comprises conducting no more than 2-30 sequencing cycles on the 2-10,000 concatemer molecules to generate a plurality of sequencing read products.

In some embodiments, only the first target barcode region of the first concatemer molecules are sequenced (e.g., FIG. 27, top). In some embodiments, at least a portion or the full length of the first target barcode of the first concatemer molecules are sequenced (e.g., FIG. 27, top). In some embodiments, the first target barcode is sequenced and a portion of the first cDNA region (or the first RNA region) of the first concatemer molecules are sequenced. In some embodiments, at least a portion of the first cDNA region (or the first RNA region) of the first concatemer molecules are sequenced.

In some embodiments, only the second target barcode region of the second concatemer molecules are sequenced (e.g., FIG. 27, bottom). In some embodiments, at least a portion or the full length of the second target barcode of the second concatemer molecules are sequenced (e.g., FIG. 27, bottom). In some embodiments, the second target barcode is sequenced and a portion of the second cDNA region (or the second RNA region) of the second concatemer molecules are sequenced. In some embodiments, at least a portion of the second cDNA region (or the second RNA region) of the second concatemer molecules are sequenced.

FIG. 27 is a schematic showing a rolling circle and sequencing workflow inside a cell, comprising generating first and second concatemers by conducting rolling circle amplification using first and second covalently closed circular molecules (respectively). The first and second concatemers are subjected to a sequencing workflow using universal sequencing primers, sequencing polymerases, and a plurality of nucleotide reagents.

In some embodiments, the sequencing of step (f) comprises contacting the plurality of concatemer molecules inside the cellular sample with (i) a plurality of universal sequencing primers, (ii) a plurality of sequencing polymerases, and (iii) a plurality of nucleotide reagents, under a condition suitable for hybridizing the plurality of universal sequencing primers to their respective universal sequencing primer binding sites on the concatemers. In some embodiments, the sequencing of step (f) further comprises conducting no more than 2-30 sequencing cycles to generate at least a first plurality of sequencing read products by sequencing at least the first target barcode region (Target BC-1), and optionally conducting no more than 2-30 sequencing cycles to generate at least a second plurality of sequencing read products by sequencing at least the second target barcode region (Target BC-2). In some embodiments, the nucleotide reagents comprise multivalent molecules, nucleotides and/or nucleotide analogs.

In some embodiments, the sequencing of step (f) comprises sequencing at least a portion of the first and second nucleic acid concatemers using an optical imaging system comprising a field-of-view (FOV) greater than 1.0 mm².

In some embodiments, in the sequencing of step (f), the plurality of first and second sequencing read products are detectable by imaging, and wherein the sequencing comprises decoding the plurality of first and second sequencing read products from the images obtained during the no more than 2-30 sequencing cycles.

In some embodiments, in the sequencing of step (f), the plurality of the first and second sequencing read products are detectable by imaging, and wherein the sequencing comprises simultaneously imaging the plurality of first and second detectable sequencing read products in the cellular sample (co-localization of the first and second sequencing read products).

In some embodiments, methods for conducting sequencing reactions that detects in situ at least two different target RNA molecules in a cellular sample further comprising step (g): removing the plurality of first sequencing read products from the first concatemer molecules and retaining the first concatemer molecules in the cellular sample, and removing the plurality of second sequencing read products from the second concatemer molecules and retaining the second concatemer molecules in the cellular sample.

In some embodiments, methods further comprising step (h): reiteratively sequencing the plurality of concatemers by repeating steps (f) and (g) at least once, wherein the sequences of the plurality of first sequencing read products confirms the presence of the first target RNA molecules in the cellular sample, and wherein the sequences of the plurality of second sequencing read products confirms the presence of the second target RNA molecules in the cellular sample.

In some embodiments, reiteratively sequencing at least one region of the concatemer comprises repeating steps (f)-(g) at least 2 times, at least 3 times, at least 4 times, at least 5 times, at least 6 times, at least 7 times, at least 8 times, at least 9 times, or at least 10 times.

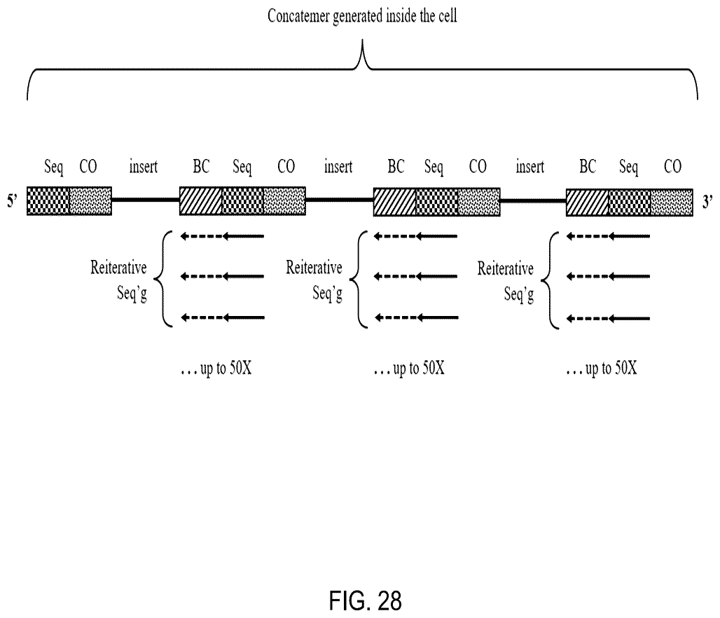

In some embodiments, reiteratively sequencing at least one region of the concatemer comprises repeating steps (f)-(g) up to 10 times, up to 20 times, up to 30 times, up to 40 times, or up to 50 times. An example of reiterative sequence is shown in a schematic in FIGS. 28-31.

FIG. 28 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC). In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include only the target barcode sequence. The plurality of first sequencing read products are removed from the concatemer, and the sequencing is repeated where no more than 30 sequencing cycles are conducted to generate another plurality of first sequencing read products (dashed arrows), where the first sequencing read products include only the target barcode sequence. The plurality of first sequencing read products are removed from the concatemer, and the sequencing is once again repeated where no more than 30 sequencing cycles are conducted to generate another plurality of first sequencing read products (dashed arrows), where the first sequencing read products include only the target barcode sequence. In some embodiments, the reiterative sequencing can be conducted up to 50 times. The sequences of all of the first sequencing read products can be determined and aligned with a first reference sequence (e.g., reference barcode sequence) to confirm the presence of the first target RNA molecules inside the cellular sample.

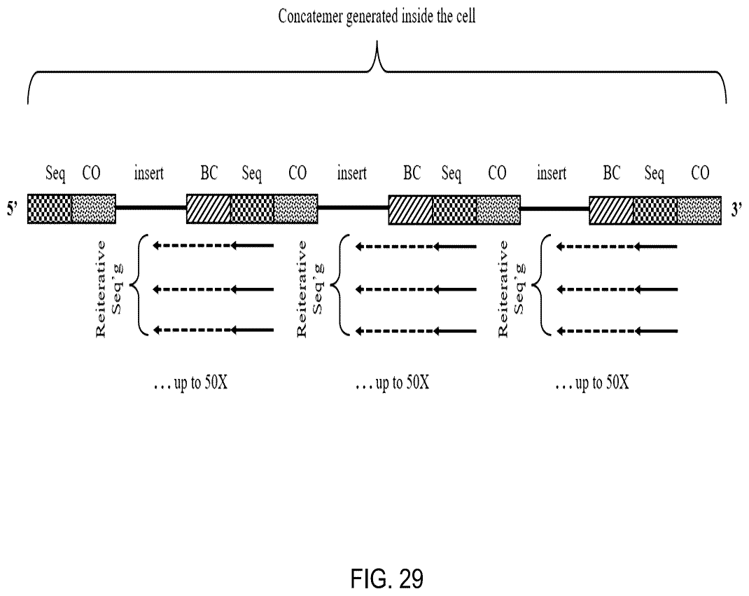

FIG. 29 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC). In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include the target barcode sequence and a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include the target barcode sequence and a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include the target barcode sequence and a portion of the insert sequence. In some embodiments, the reiterative sequencing can be conducted up to 50 times. The sequences of all of the first sequencing read products can be determined and aligned with a first reference sequence (e.g., reference barcode sequence and the insert sequence that corresponds to the target RNA) to confirm the presence of the first target RNA molecules inside the cellular sample.

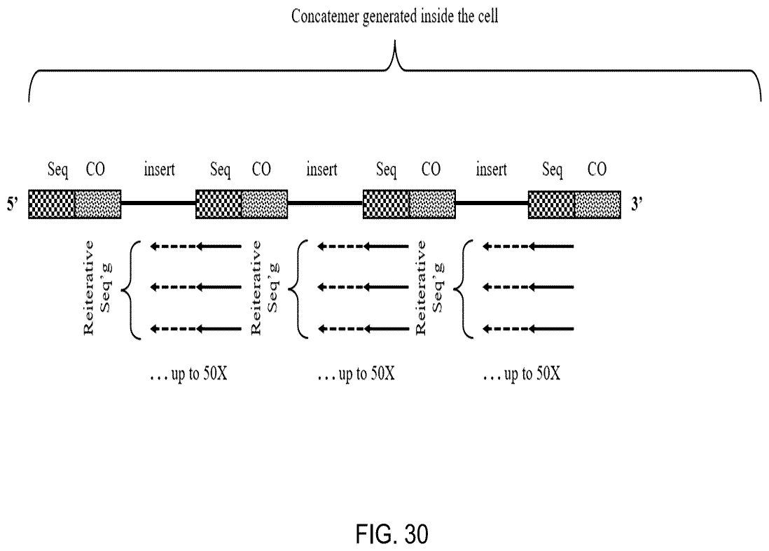

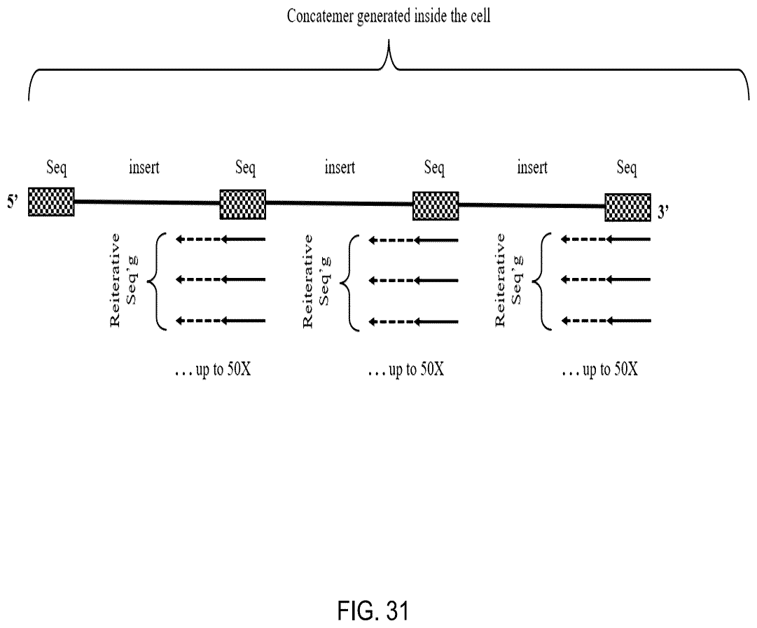

FIG. 30 is a schematic showing an exemplary workflow for sequencing a concatemer that is generated inside the cell. The concatemer includes tandem repeat units where each unit comprises: (i) a universal sequencing primer binding site (Seq), (ii) universal compaction oligonucleotide binding site (CO), and (iii) an insert sequence that corresponds to a given target cDNA. In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

FIG. 31 is a schematic showing an exemplary workflow for sequencing a concatemer that is generated inside the cell. The concatemer includes tandem repeat units where each unit comprises: (i) a universal sequencing primer binding site (Seq) and (ii) an insert sequence that corresponds to a given target cDNA. In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

In some embodiments, at least one concatemer is sequenced by conducting step (f) once (non-reiterative sequencing). In some embodiments, at least one concatemer is sequenced by conducting steps (f)-(g) once. In some embodiments, at least one concatemer is reiteratively sequenced by conducting steps (f)-(g) at least twice.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the plurality of nucleotide reagents of step (f) comprise a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to the 3′ nucleotide sugar position to form a 3′ blocked nucleotide analog. In some embodiments, the chain terminating moiety can be removed to convert the 3′ blocked nucleotide analog to an extendible nucleotide having a 3′ OH group on the sugar. In some embodiments, the labeled nucleotide analogs are linked to a different fluorophore that corresponds to the nucleo-bases adenine, cytosine, guanine, thymine or uracil, where the different fluorophores emit a fluorescent signal during the sequencing of step (f). In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and the fluorophore from the incorporated nucleotide and retaining the concatemer/sequencing primer duplex. In some embodiments, no more than 2-30 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample.

In some embodiments, the sequences of the first and second sequencing read products can be aligned after each round of generating the first and second sequencing read products which are no more than 30 bases in length, or after generating a set of reiterative sequencing read products wherein the first and second sequencing read products which are no more than 30 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, when the sequencing is conducted using different fluorescently-labeled nucleotide reagents that correspond to different nucleo-bases (e.g., A, G, C, T/U), then the images can have different color fluorescent spots co-located in the same cellular sample at different sequencing cycles.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents of step (f) comprise a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle comprises (1) contacting the concatemer/sequencing primer duplex with a first sequencing polymerase to form a complexed polymerase, (2) contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer, (3) detecting and imaging the fluorescent signal and color emitted by the bound detectably labeled multivalent molecule, (4) removing the first sequencing polymerase and the bound detectably labeled multivalent molecule, and retaining the concatemer/sequencing primer duplex, (5) contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer, and (6) removing the chain terminating moiety (e.g., unblocking) and retaining the concatemer/sequencing primer duplex. In some embodiments, no more than 2-30 sequencing cycles are conducted on the plurality of concatemers inside the cellular sample to generate a plurality of sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample. In some embodiments, the sequences of the first and second sequencing read products can be aligned after each round of generating the first and second sequencing read products which are no more than 30 bases in length, or after generating a set of reiterative sequencing read products wherein the first and second sequencing read products which are no more than 30 bases in length. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules. In some embodiments, individual cycle times can be achieved in less than 30 minutes. In some embodiments, the field of view (FOV) can exceed 1 mm² and the cycle time for scanning large area (>10 mm²) can be less than 5 minutes.

In some embodiments, when sequencing with detectably labeled multivalent molecules, step (2) in which multivalent-binding complexes are formed and step (3) in which the bound detectably labeled multivalent molecules are imaged and detected, the conditions are gentle compared to sequencing workflows that employ detectable labeled chain terminating nucleotides. For example, steps (2) and (3) can be conducted at a gentle temperature of about 35-45° C., or about 39-42° C. Steps (2) and (3) can be conducted at a gentle temperature which can help retain the compact size and shape of a DNA nanoball during multiple sequencing cycles (e.g., up to 30 cycles) which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

In some embodiments, out-of-sync phasing and/or pre-phasing events can occur during synchronized polymerase-catalyzed sequencing reactions employing detectably labeled multivalent molecules. During sequencing, a fluorescent signal can be detected which corresponds to binding of complementary nucleotide unit of a multivalent molecule to the complexed polymerase thereby forming a multivalent-binding complex. Thus, phasing and pre-phasing events can be detected and monitored using binding of labeled multivalent molecules. In some embodiments, when conducting up to 30 sequencing cycles with detectably labeled multivalent molecules, the phasing and/or pre-phasing rate can be less than about 5%, or less than about 1%, or less than about 0.01%, or less than about 0.001%. By contrast, the phasing and/or pre-phasing rates for conducting up to 30 sequencing cycles using labeled chain terminator nucleotides can be about 5%.

### Target-Specific Padlock Probes

In any of the methods described herein, the plurality of RNA or cDNA inside the cellular sample can be amplified to generate amplicons of the RNA or cDNA where the amplicons comprise concatemers. In some embodiments, the plurality of RNA or cDNA molecules inside the cellular sample can be amplified by conducting a padlock probe circularization and rolling circle amplification workflow. In some embodiments, the methods comprise contacting the plurality of RNA or cDNA molecules inside the cellular sample with a plurality of padlock probes, including a first plurality of target-specific padlock probes that hybridize with first target RNA or cDNA molecules, and a second plurality of target-specific padlock probes that hybridize with second target RNA or cDNA molecules.

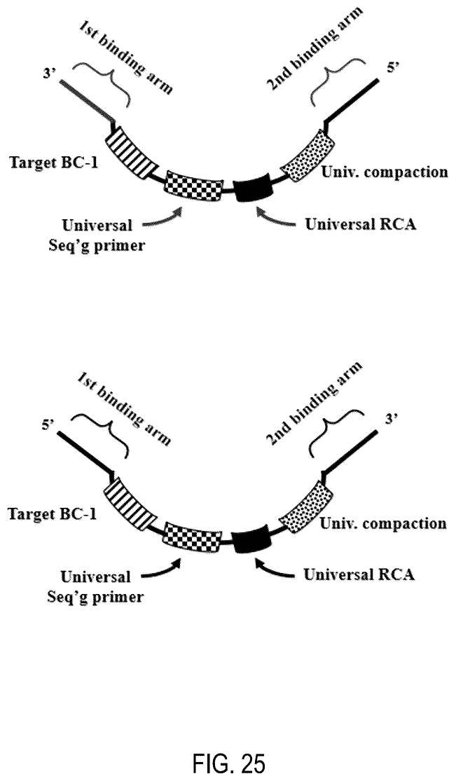

In some embodiments, the padlock probes comprise single-stranded oligonucleotides. In some embodiments, the padlock probes comprise DNA, RNA, or DNA and RNA. In some embodiments, individual padlock probes comprise an internal region between the first and second terminal regions, where the internal region comprises at least one universal adaptor sequence including a sample barcode sequence, an amplification primer binding site, a sequencing primer binding site, a compaction oligonucleotide binding site and/or a surface capture primer binding site (FIG. 25). In some embodiments, the padlock probes comprise at least one target barcode sequence that corresponds to a given target RNA or target cDNA to which the padlock probes binds. In some embodiments, the padlock probes comprise at least one unique identification sequence (e.g., unique molecular index (UMI)). In some embodiments, the padlock probes comprise at least one restriction enzyme recognition sequence.

In some embodiments, individual padlock probes comprise first and second terminal regions (e.g., first and second binding arms) that hybridize to portions of target RNA or target cDNA molecules to form a plurality of RNA-padlock probe complexes or a plurality of cDNA-padlock probe complexes, wherein individual complexes have the first and second terminal probe regions hybridized to proximal regions of an RNA or cDNA molecule to form a nick or gap between the first and second terminal probe ends. In some embodiments, the first terminal region of an individual padlock probe has a first target-specific sequence that selectively hybridizes to a first region of a target RNA or cDNA molecule, and the second terminal region of the individual padlock probe has a second target-specific sequence that selectively hybridizes to a second region of the same target RNA or cDNA molecule, where a nick or gap is formed between the hybridized first and second terminal regions, thereby circularizing the padlock probe (e.g., FIG. 26).

In some embodiments, the padlock probes comprise canonical nucleotides and/or nucleotide analogs. In some embodiments, the padlock probes are modified to confer resistance to nuclease degradation (e.g., ribonuclease degradation). For example, the padlock probes comprise at least one phosphorothioate diester bond at their 5′ ends which can render the padlock probes resistant to nuclease degradation. In some embodiments, the padlock probes comprise 2-5 or more consecutive phosphorothioate diester bonds at their 5′ ends. In some embodiments, the padlock probes comprise at least one ribonucleotide and/or at least one 2′-O-methyl, 2′-O-methoxyethyl (MOE), 2′ fluoro-base nucleotide. In some embodiments, the padlock probes comprise phosphorylated 3′ ends. In some embodiments, the padlock probes comprise at least one locked nucleic acid (LNA) base. In some embodiments, the padlock probes comprise a phosphorylated 5′ end (e.g., using a polynucleotide kinase).

FIG. 25 is a schematic showing exemplary embodiments of padlock probes. In some embodiments, a padlock probe comprises a single-stranded nucleic acid molecule having two terminal regions (e.g., first and second binding arms) and an internal region. In some embodiments, the first terminal region of an individual padlock probe has a first target-specific sequence that selectively hybridizes to a first region of a target RNA or target cDNA molecule, and the second terminal region of the individual padlock probe has a second target-specific sequence that selectively hybridizes to a second region of the same target RNA or target cDNA molecule. In some embodiments, the internal region of a padlock comprises a target barcode sequence (e.g., Target BC-1 or Target BC-2, left and right schematics respectively) which corresponds to a given target RNA or target cDNA. In some embodiments, the target barcode sequence uniquely identifies the target RNA or target cDNA. In some embodiments, the internal region of a padlock comprises a universal primer binding site for a sequencing primer (or a complementary sequence thereof). In some embodiments, the internal region of a padlock comprises a universal primer binding site for a rolling circle amplification primer (or a complementary sequence thereof). In some embodiments, the internal region of a padlock comprises a universal binding site for a compaction oligonucleotide binding (or a complementary sequence thereof). In some embodiments, the internal region of a padlock probe includes a target barcode sequence and at least one universal primer binding site (e.g., for binding a sequencing primer, for binding a rolling circle amplification primer and/or for binding a compaction oligonucleotide) in any arrangement and orientation (FIG. 25, top and bottom).

In some embodiments, individual padlock probes in a set of padlock probes (e.g., a plurality of padlock probes) comprise first and second terminal regions that hybridize to the same target regions of the target RNA or cDNA molecules to form a plurality of RNA-padlock probe complexes or a plurality of cDNA-padlock probe complexes having the same RNA or cDNA sequence.

In some embodiments, a set of padlock probes (e.g., a plurality of padlock probes) comprise at least two sub-sets of padlock probes. In some embodiments, individual padlock probes in a first sub-set of padlock probes comprise first and second terminal regions that hybridize to the same target regions (e.g., a first target region) of the target RNA or cDNA molecules to form a first plurality of RNA-padlock probe complexes or a first plurality of cDNA-padlock probe complexes having the same RNA or cDNA sequence. In some embodiments, individual padlock probes in a second sub-set of padlock probes comprise first and second terminal regions that hybridize to the same target regions (e.g., a second target region) of the target RNA or cDNA molecules to form a second plurality of RNA-padlock probe complexes or a second plurality of cDNA-padlock probe complexes having the same cDNA sequence. In some embodiments, the first and second sub-sets of padlock probes hybridize to different target regions of the same target RNA or cDNA molecules. In some embodiments, the first and second sub-sets of padlock probes hybridize to different target regions of different target RNA or cDNA molecules. In some embodiments, the set of padlock probes comprise 2-10 sub-sets of padlock probes, or 10-25 sub-sets of padlock probes, or 25-50 sub-sets of padlock probes, or up to 100 sub-sets of padlock probes. In some embodiments, the set of padlock probes comprise at least 100 sub-sets of padlock probes, at least 500 sub-sets of padlock probes, at least 1000 sub-sets of padlock probes, at least 10,000 sub-sets of padlock probes, or more sub-sets of padlock probes.

In some embodiments, the nicks can be enzymatically ligated to generate covalently closed circular padlock probes. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the size of the gap between the hybridized first and second terminal regions is 1-25 bases. The 3′OH end of hybridized padlock probe can serve as an initiation site for a polymerase-catalyzed fill-in reaction (e.g., gap fill-in reaction) using the target cDNA molecule (or the target RNA molecule) as a template. After the fill-in reaction, the remaining nick can be enzymatically ligated to generate covalently closed circular padlock probes.

In some embodiments, the gap-filling reaction comprises contacting the circularized padlock probe with a DNA polymerase and a plurality of nucleotides. In some embodiments, the DNA polymerase comprises E. coli DNA polymerase I, Klenow fragment of E. coli DNA polymerase I, T7 DNA polymerase, or T4 DNA polymerase. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

### Rolling Circle Amplification

In any of the methods described herein, the plurality of covalently closed circular padlock probes can be subjected to a rolling circle amplification reaction to generate a plurality of concatemer molecules each having two or more tandem copies of a unit wherein the unit comprises a target sequence that corresponds to a target RNA molecules and any additional sequence(s) carried by the padlock probes including universal adaptor sequence(s), unique molecular index sequence(s) and/or restriction enzyme recognition sequence(s).

In some embodiments, the rolling circle amplification reaction comprises contacting the covalently closed circularized padlock probes with an amplification primer (e.g., a universal rolling circle amplification primer), a strand-displacing DNA polymerase, and a plurality of nucleotides, under a condition suitable for hybridizing individual amplification primers to a covalently closed padlock probe, and under a condition suitable for conducting primer extension using the covalently closed padlock probe as a template molecule to generate a nucleic acid concatemer. In some embodiments, the plurality of nucleotides in the rolling circle amplification reaction comprise any mixture of two or more of dATP, dGTP, dCTP, dTTP and/or dUTP. In some embodiments, any of the rolling circle amplification reactions described herein can be conducted in the presence or in the absence of a plurality of compaction oligonucleotides.

In some embodiments, when the rolling circle amplification reaction includes a plurality of nucleotide which includes dUTP, the resulting concatemer can be cross-linked to a cross-linking reactive group by treating the cellular sample with a succinimide ester (NHS), maleimide (Sulfo-SMCC), imidoester (DMP), carbodiimide (DCC, EDC) or phenyl azide. In some embodiments, polymerization of the cross-linking reactive group can be initiated with light or UV light. In some embodiments, the resulting concatemer can be cross-linked to a matrix by treating the cellular sample with a cross-linked agarose, cross-linked dextran or cross-linked polyethylene glycol (PEG), polyacrylamide, cellulose alginate or polyamide. In some embodiments, the PEG comprises a sulfo-NHS ester moiety at one or both ends, for example a PEGylated bis(sulfosuccinimidyl)suberate) (e.g., BS(PEG)₉ from Thermo Fisher Scientific, catalog No. 21582).

In some embodiments, the rolling circle amplification reaction can be conducted at a constant temperature (e.g., isothermal) wherein the constant temperature is at room temperature to about 30° C., or about 30-40° C., or about 40-50° C., or about 50-65° C.

In some embodiments, the DNA polymerase having a strand displacing activity can be selected from a group consisting of phi29 DNA polymerase, large fragment of Bst DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In some embodiments, the phi29 DNA polymerase can be wild type phi29 DNA polymerase (e.g., MagniPhi from Expedeon), or variant EquiPhi29 DNA polymerase (e.g., from Thermo Fisher Scientific), and chimeric QualiPhi DNA polymerase (e.g., from4basebio).

In some embodiments, the rolling circle amplification primers can be modified to increase resistance to nuclease degradation. In some embodiments, the rolling circle amplification primers comprise at least one phosphorothioate diester bond at their 5′ ends which can render the amplification primers resistant to exonuclease degradation. In some embodiments, the rolling circle amplification primers comprise 2-5 or more consecutive phosphorothioate diester bonds at their 5′ ends. In some embodiments, the rolling circle amplification primers comprise at least one ribonucleotide and/or at least one 2′-O-methyl or 2′-O-methoxyethyl (MOE) nucleotide.

In some embodiments, the rolling circle amplification reaction can be conducted in the presence of a plurality of compaction oligonucleotides which, when hybridized to a concatemer molecule, compacts the size and/or shape of the concatemer to form a compact nanoball. In some embodiments, the compaction oligonucleotides comprise single stranded oligonucleotides having a first region at one end that hybridizes to a portion of a concatemer molecule and a second region at the other end that hybridizes to another portion of the same concatemer molecule, where hybridization of the compaction oligonucleotide to a given concatemer compacts the size and/or shape of the concatemer.

The compaction oligonucleotides include a 5′ region, an optional internal region (intervening region), and a 3′ region. The 5′ and 3′ regions of the compaction oligonucleotide can hybridize to any portions of the concatemer. The 5′ and 3′ regions of the compaction oligonucleotide can hybridize to different portions of the concatemer to pull together distal portions of the concatemer causing compaction of the concatemer to form a DNA nanoball. For example, the 5′ region of the compaction oligonucleotide is designed to hybridize to a first portion of the concatemer molecule (e.g., a universal compaction oligonucleotide binding site), and the 3′ region of the compaction oligonucleotide is designed to hybridized to a second portion of the concatemer molecule (e.g., a universal compaction oligonucleotide binding site). Inclusion of compaction oligonucleotides during RCA can promote formation of DNA nanoballs having tighter size and shape compared to concatemers generated in the absence of the compaction oligonucleotides. The compact and stable characteristics of the DNA nanoballs improves in situ sequencing accuracy by increasing signal intensity and the nanoballs retain their shape and size during multiple sequencing cycles.

In some embodiments, the compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between the 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule.

In some embodiments, the 5′ region of the compaction oligonucleotide can have the same sequence as the 3′ region. The 5′ region of the compaction oligonucleotide can have a sequence that is different from the 3′ region. In some embodiments, the 3′ region of the compaction oligonucleotide can have a sequence that is a reverse sequence of the 5′ region. In some embodiments, the 5′ region of the compaction oligonucleotide can have a sequence that is a reverse sequence of the 3′ region.

In some embodiments, the 3′ region of any of the compaction oligonucleotides can include an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides comprise one or more modified bases or linkages at their 5′ or 3′ ends to confer certain functionalities. In some embodiments, the compaction oligonucleotides comprise at least one phosphorothioate linkages at their 5′ and/or 3′ ends to confer exonuclease resistance. In some embodiments, at least one nucleotide at or near the 3′ end comprises a 2′ fluoro base which confers exonuclease resistance. In some embodiments, the 3′ end of the compaction oligonucleotides comprise at least one 2′-O-methyl RNA base which blocks polymerase-catalyzed extension. For example, the 3′ end of the compaction oligonucleotide comprises three bases comprising 2′-O-methyl RNA base (e.g., designated mUmUmU). In some embodiments, the compaction oligonucleotides comprise a 3′ inverted dT at their 3′ ends which blocks polymerase-catalyzed extension. In some embodiments, the compaction oligonucleotides comprise 3′ phosphorylation which blocks polymerase-catalyzed extension. In some embodiments, the internal region of the compaction oligonucleotides comprise at least one locked nucleic acid (LNA) which increases the thermal stability of duplexes formed by hybridizing a compaction oligonucleotide to a concatemer molecule. In some embodiments, the compaction oligonucleotides comprise a phosphorylated 5′ end (e.g., using a polynucleotide kinase).

In some embodiments, the compaction oligonucleotide comprises the sequence 5′-CATGTAATGCACGTACTTTCAGGGTAAACATGTAATGC ACGTACTTTCAGGGT-3′ (SEQ ID NO: 1). In some embodiments, the compaction oligonucleotides includes an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure. The guanine tetrad structure can be stabilized via Hoogsteen hydrogen bonding. The guanine tetrad structure can be stabilized by a central cation including potassium, sodium, lithium, rubidium or cesium.

At least one compaction oligonucleotide can form a guanine tetrad and hybridize to the universal binding sequences in a concatemer which can cause the concatemer to fold to form an intramolecular G-quadruplex structure. The concatemers can self-collapse to form compact nanoballs. Formation of the guanine tetrads and G-quadruplexes in the nanoballs may increase the stability of the nanoballs to retain their compact size and shape which can withstand changes in pH, temperature and/or repeated flows of reagents during sequencing inside the cellular sample.

In some embodiments, the plurality of compaction oligonucleotides in the rolling circle amplification reaction have the same sequence. Alternatively, the plurality of compaction oligonucleotides in the rolling circle amplification reaction comprise a mixture of two or more different populations of compaction oligonucleotides having different sequences.

In some embodiment, the immobilized concatemer template molecule can self-collapse into a compact nucleic acid nanoball. The nanoballs can be imaged and a FWHM measurement can be obtained to give the shape/size of the nanoballs.

In some embodiments, inclusion of compaction oligonucleotides in the rolling circle amplification reaction can promote collapsing of a concatemer into a DNA nanoball. Conducting RCA with compaction oligonucleotides helps retain the compact size and shape of a DNA nanoball during multiple sequencing cycles which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

The single-stranded concatemers collapse into compact DNA nanoballs, where each nanoball carries numerous tandem copies of a polynucleotide unit along their lengths, where the polynucleotide unit includes a sequence-of-interest (e.g., that corresponds to target RNA or target cDNA) and at least a universal sequencing primer binding site. Each polynucleotide unit can bind a sequencing primer, a sequencing polymerase and a detectably-labeled nucleotide reagent (e.g., detectably labeled multivalent molecules), to form a detectable sequencing complex (e.g., a detectable ternary complex). Each nanoball carries numerous detectable sequencing complexes. Thus, the compact nature of the nanoballs increases the local concentration of detectably-labeled nucleotide reagents that are used during the sequencing workflow which increases the signal intensity emitted from a nanoball to give a discrete detectable signal which can be imaged as a fluorescent spot inside the cellular sample. Each spot corresponds to a concatemer and each concatemer corresponds to a target RNA molecule in the cellular sample. Multiple spots can be detected and imaged simultaneously in the cellular sample. The DNA nanoballs having compact shape and size that produce increased signal intensity and color differentiation during sequencing.

### Cellular Sample

In any of the methods described herein, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample comprise one or more living cells or non-living cells.

In some embodiments, the cellular sample can be obtained from a virus, fungus, prokaryote or eukaryote. In some embodiments, the cellular sample can be obtained from an animal, insect or plant. In some embodiments, the cellular sample comprises one or more virally-infected cells.

In some embodiments, the cellular sample can be obtained from any organism including human, simian, ape, canine, feline, bovine, equine, murine, porcine, caprine, lupine, ranine, piscine, plant, insect or bacteria.

In some embodiments, the cellular sample can be obtained from any organ including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs.

In any of the methods described herein, the cellular sample harbors a plurality of RNA which include target RNA and non-target RNA. In some embodiments, cells typically produce RNA by gene expression which includes transcription of DNA (e.g., genomic DNA) into RNA molecules. The transcribed RNA can undergo splicing or may not be spliced. The transcribed RNA can be translated into a polypeptide (e.g., coding RNA), or do not undergo translation but can be processed into tRNA or rRNA (e.g., non-coding RNA).

In some embodiments, the plurality of RNA harbored by the cellular sample includes target and non-target RNA. In some embodiments, the plurality of RNA harbored by the cellular sample comprises wild type RNA, mutant RNA or splice variant RNA. In some embodiments, the plurality of RNA harbored by the cellular sample comprises pre-spliced RNA, partially spliced RNA, or fully spliced RNA. In some embodiments, the plurality of RNA harbored by the cellular sample comprises coding RNA, non-coding RNA, mRNA, tRNA, rRNA, microRNA (miRNA), mature microRNA, or immature microRNA. In some embodiments, the plurality of RNA harbored by the cellular sample comprises housekeeping RNA, cell-specific RNA, tissue-specific RNA or disease-specific RNA. In some embodiments, the plurality of RNA harbored by the cellular sample comprises RNA expressed by one or more cells in response to a stimulus such as heat, light, a chemical or a drug. In some embodiments, the plurality of RNA harbored by the cellular sample comprises RNA found in healthy cells or diseased cells. In some embodiments, the plurality of RNA harbored by the cellular sample comprises RNA transcribed from transgenic DNA sequences that are introduced into the cellular sample using recombinant DNA procedures. For example, the RNA can be transcribed from a transgenic DNA sequence that is controlled by an inducible or constitutive promoter sequence. In some embodiments, the plurality of RNA harbored by the cellular sample comprises RNA that is transcribed from DNA sequences that are not transgenic.

### Culturing Cellular Sample on a Support

In any of the methods described herein, the cellular sample can be cultured on the support. In some embodiments, the methods comprise culturing the cellular sample on the support under a condition suitable for expanding the cellular sample for 2-10 generations or more. The cultured cellular sample can generate a colony of cells. In some embodiments, the methods comprise culturing the cellular sample to confluence or non-confluence. In some embodiments, the methods comprise culturing the cellular sample on the support in a simple or complex cell culture media. For example, the cell culture media comprises D-MEM high glucose (e.g., from Thermo Fisher Scientific, catalog No. 11965118), fetal bovine serum (e.g., 10% FBS; for example from Thermo Fisher Scientific, catalog No. A3160402), MEM non-essential amino acids (e.g., 0.1 mM MEM, for example from Thermo Fisher Scientific, catalog No. 11140050), L-glutamine (e.g., 6 mM L-glutamine, for example from Thermo Fisher Scientific, catalog No. A2916801), MEM sodium pyruvate (e.g., 1 mM sodium pyruvate, for example from Thermo Fisher Scientific, catalog No. 11360070), and an antibiotic (e.g., 1% penicillin-streptomycin-glutamine, for example from Thermo Fisher, catalog No. 10378016). In some embodiments, the methods comprise culturing the cellular sample at a humidity and temperature that is suitable for culturing the cell(s) on the support. Exemplary suitable conditions comprise approximately 37° C. with a humidified atmosphere of approximately 5-10% carbon dioxide in air. The cellular sample can be cultured with suitable aeration with oxygen and/or nitrogen.

### Simple Cell Media

In any of the methods described herein, the term “simple cell media” or related terms refers to a cell media that typically lacks ingredients to support cell growth and/or proliferation in culture. Simple cell media can be used for example to wash, suspend, or dilute the cellular sample. Simple cell media can be mixed with certain ingredients to prepare a cell media that can support cell growth and/or proliferation in culture. A simple cell media comprises any one or any combination of two or more of a buffer, a phosphate compound, a sodium compound, a potassium compound, a calcium compound, a magnesium compound and/or glucose. In some embodiments, the simple cell media comprises PBS (phosphate buffered saline), DPBS (Dulbecco's phosphate-buffered saline), HBSS (Hank's balanced salt solution), DMEM (Dulbecco's Modified Eagle's Medium), EMEM (Eagle's Minimum Essential Medium), and/or EBSS. In some embodiments, the cellular sample can be placed in a simple cell media prior to or during the step of conducting any of the nucleic acid methods described herein.

### Complex Cell Media

In any of the methods described herein, the term “complex cell media” or related terms refers to a cell media that can be used to support cell growth and/or proliferation in culture without supplementation or additives. Complex cell media can include any combination of two or more of a buffering system (e.g., HEPES), inorganic salt(s), amino acid(s), protein(s), polypeptide(s), carbohydrate(s), fatty acid(s), lipid(s), purine(s) and their derivatives (e.g., hypoxanthine), pyrimidine(s) and their derivatives, and/or trace element(s). Complex cell media includes fluids obtained from a fluid or tissue extract. Complex cell media includes artificial cell media. In some embodiments, complex cell media can be a serum-containing media, for example complex cell media includes fluids such as fetal bovine serum, blood plasma, blood serum, lymph fluid, human placental cord serum and amniotic fluid. In some embodiments, complex cell media can be a serum-free media, which are typically (but not necessarily) defined cell culture media. In some embodiments, complex cell media can be a chemically-defined media which typically (but not necessarily) include recombinant polypeptides, and ultra-pure inorganic and/or organic compounds. In some embodiments, complex cell media can be a protein-free media which include for example MEM (minimal essential media) and RPMI-1640 (Roswell Park Memorial Institute). In some embodiments, the complex cell media comprises IMDM (Iscove's Modified Dulbecco's Medium. In some embodiments, the complex cell media comprises DMEM (Dulbecco's Modified Eagle's Medium). In some embodiments, the cellular sample can be placed in a complex cell media prior to or during the step of conducting any of the nucleic acid methods described herein.

In any of the methods described herein, the cellular sample comprises a fixed cellular sample. In some embodiments, the cellular sample can be treated with a fixation reagent (e.g., a fixing reagent) that preserves the cell and its contents to inhibit degradation and can inhibit cell lysis. For example, the fixation reagent can preserve RNA harbored by the cellular sample. In some embodiments, the fixation reagent inhibits loss of nucleic acids from the cellular sample.

In some embodiments, the fixation reagent can cross-link the RNA to prevent the RNA from escaping the cellular sample. In some embodiments, a cross-linking fixation reagent comprises any combination of an aldehyde, formaldehyde, paraformaldehyde, formalin, glutaraldehyde, imidoesters, N-hydroxysuccinimide esters (NHS) and/or glyoxal (a bifunctional aldehyde).

In some embodiments, the fixation reagent comprises at least one alcohol, including methanol or ethanol. In some embodiments, the fixation reagent comprises at least one ketone, including acetone. In some embodiments, the fixation reagent comprises acetic acid, glacial acetic acid and/or picric acid. In some embodiments, the fixation reagent comprises mercuric chloride. In some embodiments, the fixation reagent comprises a zinc salt comprising zinc sulphate or zinc chloride. In some embodiments, the fixation reagent can denature polypeptides.

In some embodiments, the fixation reagent comprises 4% w/v of paraformaldehyde to water/PBS. In some embodiments, the fixation reagent comprises 10% of 35% formaldehyde at a neutral pH. In some embodiments, the fixation reagent comprises 2% v/v of glutaraldehyde to water/PBS. In some embodiments, the fixation reagent comprises 25% of 37% formaldehyde solution, 70% picric acid and 5% acetic acid.

In some embodiments, the cellular sample can be fixed on the support with 4% paraformaldehyde for about 30-60 minutes and washed with PBS.

In some embodiments, the cellular sample can be stained, de-stained or un-stained.

In any of the methods described herein, the cellular sample comprises a permeabilized cellular sample. In some embodiments, the methods comprise treating the cellular sample with a permeabilization reagent that alters the cell membrane to permit penetration of experimental reagents into the cells. For example, the permeabilization reagent removes membrane lipids from the cell membrane. In some embodiments, the cellular sample can be treated with a permeabilization reagent which comprises any combination of an organic solvent, detergent, chemical compound, cross-linking agent and/or enzyme. In some embodiments, the organic solvents comprise acetone, ethanol, and methanol. In some embodiments, the detergents comprise saponin, Triton X-100, Tween-20, sodium dodecyl sulfate (SDS), an N-lauroylsarcosine sodium salt solution, or a nonionic polyoxyethylene surfactant (e.g., NP40). In some embodiments, the cross-linking agent comprises paraformaldehyde. In some embodiments, the enzyme comprises trypsin, pepsin or protease (e.g. proteinase K). In some embodiments, the cells can be permeabilized using an alkaline condition, or an acidic condition with a protease enzyme. In some embodiments, the permeabilization reagent comprises water and/or PBS.

For example, the fixed cells can be permeabilized with 70% ethanol for about 30-60 minutes, and the permeabilizing reagent can be exchanged with PBS-T (e.g., PBS with 0.05% Tween-20). In some embodiments, the cells can be post-fixed with 3% paraformaldehyde and 0.1% glutaraldehyde for about 30-60 minutes, and washed with PBS-T multiple times.

In any of the methods described herein, the cellular sample is infused with a swellable polyelectrolyte hydrogel (U.S. Pat. No. 10,309,879 and Chen 2015 Science 347:543, the contents of these documents are incorporated by reference in their entireties). In some embodiments, a fixed and permeabilized cellular sample can be infused with sodium acrylate, acrylamide and a cross-linker N—N′-methylenebisacrylamide. In some embodiments, ammonium persulfate (APS) initiator and tetramethylethylenediamine (TEMED) accelerator were infused to achieve polymerization. In some embodiments, the cellular sample can be infused with proteinase K for proteolysis and incubated in a digestion buffer. In some embodiments, the gel inside the cellular sample can be swelled by addition of water.

In any of the methods described herein, the plurality of RNAs inside cellular sample can be converted to cDNA. In some embodiments, the methods comprise contacting the plurality of RNA inside the fixed and permeabilized cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample. In some embodiments, synthesis of second strand cDNA molecules is omitted. In some embodiments, the RNA inside the cellular sample is not converted into cDNA, where the RNA is hybridized to target-specific padlock probes.

In some embodiments, the reverse transcriptase enzyme exhibits RNA-dependent DNA polymerase activity. In some embodiments, the reverse transcriptase enzyme comprises a reverse transcriptase enzyme from AMV (avian myeloblastosis virus), M-MuLV (moloney murine leukemia virus), or HIV (human immunodeficiency virus). In some embodiment, the reverse transcriptase enzyme comprises a recombinant enzyme that exhibits reduced RNase H activity, for example REVERTAID (e.g., from Thermo Fisher Scientific, catalog No. EP0441). In some embodiments, the reverse transcriptase can be a commercially-available enzyme, including MULTISCRIBE (e.g., from Thermo Fisher Scientific, catalog #4311235), THERMOSCRIPT (e.g., from Thermo Fisher Scientific, catalog #12236-014), or ARRAYSCRIPT (e.g., from Ambion, catalog No. AM2048). In some embodiments, the reverse transcriptase enzyme comprises SUPERSCRIPT II (e.g., catalog No. 18064014), SUPERSCRIPT III (e.g., catalogNo. 18080044), or SUPERSCRIPT IV enzymes (e.g., catalog No. 18090010) (all SUPERSCRIPT enzymes from Invitrogen). In some embodiments, the reverse transcription reaction can include an RNase inhibitor.

In some embodiments, the reverse transcription primers comprise a single-stranded oligonucleotide comprising DNA, RNA, or chimeric DNA/RNA. In some embodiments, the reverse transcription primers Any combination of adenine (A), thymine (T), guanine (G), cytosine (C), uracil (U) and/or inosine (I). In some embodiments, the reverse transcription primers can be any length, for example 5-25 bases, or 25-50 bases, or 50-75 bases, or 75-100 bases in length or longer. The reverse transcription primers each comprise a 5′ end and 3′ end. In some embodiments, the 3′ end of the reverse transcription primers can include a 3′ OH moiety which serves as a nucleotide polymerization initiation site in a polymerase-catalyzed primer extension reaction. In some embodiments, the 3′ end of the reverse transcription primers have a chain terminating moiety which blocks a polymerase-catalyzed primer extension reaction. The chain terminating moiety can be removed to convert the 3′ sugar position to an extendible 3′OH.

In some embodiments, the reverse transcription primers are modified to confer resistance to nuclease degradation (e.g., ribonuclease degradation). For example, the reverse transcription primers comprise at least one phosphorothioate diester bond at their 5′ ends which can render the reverse transcription primers resistant to nuclease degradation. In some embodiments, the reverse transcription primers comprise 2-5 or more consecutive phosphorothioate diester bonds at their 5′ ends. In some embodiments, the plurality of reverse transcription primers comprise at least one ribonucleotide and/or at least one 2′-O-methyl, 2′-O-methoxyethyl (MOE), 2′ fluoro-base nucleotide. In some embodiments, the reverse transcription primers comprise phosphorylated 3′ ends. In some embodiments, the reverse transcription primers comprise locked nucleic acid (LNA) bases. In some embodiments, the reverse transcription primers comprise a phosphorylated 5′ end (e.g., using a polynucleotide kinase).

In some embodiments, the entire length of a reverse transcription primer can hybridize to a portion of an RNA molecule. In some embodiments, individual reverse transcription primers comprise a 3′ region having a sequence that hybridizes to a portion of an RNA molecule and a 5′ region that carries a tail that does not hybridize to an RNA molecule. In some embodiments, the 5′ tail comprises a universal adaptor sequence including any one or any combination of two or more of a sample barcode sequence, an amplification primer binding site, a sequencing primer binding site, a compaction oligonucleotide binding site and/or a surface capture primer binding site. In some embodiments, the 5′ tail comprises a unique identification sequence (e.g., unique molecular index (UMI). In some embodiments, the 5′ tail comprises a restriction enzyme recognition sequence. In some embodiments, individual reverse transcription primers comprise at least a portion of the 3′ region having a homopolymer sequence, for example poly-A, poly-T, poly-C, poly-G or poly-U. In some embodiments, the reverse transcription primers can hybridize to any portion of an RNA molecule, including the 5′ or the 3′ end of the RNA molecule, or an internal portion of the RNA molecule.

In some embodiments, the plurality of reverse transcription primers comprises a first sub-population of target-specific reverse transcription primers that hybridize selectively to the first target RNA (e.g., targeted transcriptomics). In some embodiments, the plurality of reverse transcription primers further comprise a second sub-population of target-specific reverse transcription primers that hybridize selectively to the second target RNA. In some embodiments, the target-specific reverse transcription primers comprise a pre-determined sequence at the 3′ region which hybridizes to a target RNA molecule. In some embodiments, the pre-determined sequence portion of the reverse transcription primers can be 4-20 bases, or 20-40 bases, or 40-50 bases in length.

In some embodiments, the first sub-population of target-specific reverse transcription primers can selectively hybridize to an RNA transcribed in the cellular sample by a housekeeping gene. In some embodiments, selection of the housekeeping gene may be dependent upon the type of cellular sample to be used for the in situ methods described herein. Exemplary housekeeping genes include glyceraldehyde-3-phosphate dehydrogenase (GAPDH), beta-actins (ACTB), tubulins, PPIA (peptidyl-prolyl cis-trans isomerase), NME4 (NME/NM23 nucleoside diphosphate kinase 4), SMARCAL1 (SWI/SNF related matrix associated actin dependent regulator of chromatin, subfamily A like 1), and POMK (protein-O-mannose kinase). The skilled artisan can design the first sub-population of target-specific reverse transcription primers to hybridize to RNA transcripts from any of the numerous housekeeping genes.

In some embodiments, the second sub-population of target-specific reverse transcription primers can selectively hybridize to an RNA transcribed from a gene that is expressed in the cellular sample being examined (e.g., a cell-specific or tissue-specific RNA).

In some embodiments, the plurality of reverse transcription primers comprises a first sub-population of random-sequence reverse transcription primers that hybridize to the first target RNA (e.g., whole transcriptomics). In some embodiments, the plurality of reverse transcription primers further comprises a second sub-population of random-sequence reverse transcription primers that hybridize to the second target RNA. In some embodiments, the reverse transcription primers comprise a random and/or degenerate sequence at the 3′ region which hybridizes to an RNA molecule. In some embodiments, the random-sequence or the degenerate-sequence portion of the reverse transcription primers can be 4-20 bases, or 20-40 bases, or 40-50 bases in length.

### Sequencing Polymerases

In any of the methods described herein, sequencing polymerases can be used for conducting sequencing reactions. In some embodiments, the sequencing polymerase(s) is/are capable of binding and incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Sequencing-by-Binding

In any of the methods described herein, the sequencing comprises conducting sequencing-by-binding (SBB) reactions inside the cellular sample, where the cDNA amplicons are the concatemer molecules. In some embodiments, the sequencing-by-binding (SBB) procedure employs non-labeled chain-terminating nucleotides. In some embodiments, a cycle of sequencing-by-binding (SBB) comprises the steps of (a) sequentially contacting a primed concatemer (e.g., a concatemer annealed to a plurality of sequencing primers) with at least two separate mixtures under ternary complex stabilizing conditions, wherein the at least two separate mixtures each include a polymerase and a nucleotide, whereby the sequentially contacting results in the primed concatemer being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed concatemer, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed concatemer after step (b), thereby producing an extended primer; and (e) repeating steps (a) through (d) at least once on the primed concatemer that comprises the extended primer. Exemplary sequencing-by-binding methods are described in U.S. Pat. Nos. 10,246,744 and 10,731,141 (where the contents of both patents are hereby incorporated by reference in their entireties).

### Nucleotides and Chain-Terminating Nucleotides

In any of the methods described herein, any of the sequencing methods described herein can employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tertbutyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Supports and Coatings

In any of the methods described herein, the solid support comprises a flowcell having a coating that promotes cell adhesion. In some embodiments, the flowcell comprises a support which can be a planar or non-planar support. The support can be solid or semi-solid. In some embodiments, the support can be porous, semi-porous or non-porous. The support can be made of any material such as glass, plastic or a polymer material. In some embodiments, the surface of the support can be coated with one or more compounds to produce a passivated layer on the support (FIG. 21). In some embodiments, the passivated layer forms a porous or semi-porous layer. In some embodiments, the support is coated with a lysine compound, poly-lysine compound, arginine compound or an amino-terminated compound. The support can be coated with an unbranched compound, a branched compound, or a mixture of unbranched and branched compounds. In some embodiments, the support is coated with surface primers for capturing nucleic acids from the cellular sample. Alternatively, the support lacks surface primers.

While preferred aspects of the present disclosure have been shown and described herein, it will be obvious to those skilled in the art that such aspects are provided by way of example only. The breadth and scope of this disclosure should not be limited by any of the above-described exemplary aspects. Numerous variations, changes, and substitutions will now occur to those skilled in the art without departing from the present disclosure. It should be understood that various alternatives to the aspects of the present disclosure described herein may be employed in practicing the aspects. It is intended that the following claims define the scope of the present disclosure and that methods and structures within the scope of these claims and their equivalents be covered thereby.

1. A method for phasing and prephasing correction in sequencing analysis, comprising:
determining, by a processor, corrected image intensities of a plurality of polonies of flow cell images in a cycle N, Ipc(N), based on a cycle N−1 phasing coefficient, pN−1, a cycle N−1 prephasing coefficient, ppN−1, or both the cycle N−1 phasing coefficient and the cycle N−1 prephasing coefficient of the plurality of polonies from a cycle N−1;
obtaining, by the processor, base calls in the cycle N based on the corrected image intensities of the plurality of polonies in the cycle N, Ipc(N);
selecting, by the processor, a set of polonies from the plurality of polonies based on the base calls in the cycle N based on the base calls in the cycle N;
determining, by the processor, a cycle N phasing coefficient, pN, a cycle N prephasing coefficient, ppN, or both the cycle N phasing coefficient and the cycle N prephasing coefficient based on image intensities of the set of selected polonies for the cycle N; and
updating, by the processor, image intensities of the plurality of polonies in cycle N, I(N), using updated and corrected image intensities, Ipc_n(N), wherein Ipc_n(N) is obtained based on the cycle N phasing coefficient, pN, the cycle N prephasing coefficient, ppN, or both.

2-8. (canceled)

9. The method of claim 1, further comprising:
determining updated and corrected image intensities, Ipc_n(N), based on the cycle N phasing coefficient, pN, the cycle N prephasing coefficient, ppN, or both.

10. (canceled)

11. The method of claim 1, further comprising:
generating normalized image intensities, Inorm(N), by normalizing the corrected image intensities in cycle N, Ipc(N).

12. (canceled)

13. The method of claim 1, further comprising:
generating normalized image intensities, Inorm(N+1), by normalizing the corrected image intensities in cycle N+1, Ipc(N+1).

14. (canceled)

15. The method of claim 11, further comprising:
updating the normalized image intensities, Inorm(N) using corrected normalized image intensities, Inorm_n(N).

16. The method of claim 15, further comprising:
generating the corrected normalized image intensities, Inorm_n(N), by dividing the updated and corrected image intensities, Ipc_n(N), by a normalization factor.

17. (canceled)

18. (canceled)

19. The method of claim 1, wherein determining the corrected image intensities in cycle N, Ipc(N), is further based on the image intensities of the plurality of polonies in cycle N, I(N).

20. (canceled)

21. (canceled)

22. The method of claim 1, wherein the determining the corrected image intensities in cycle N+1, Ipc(N+1), is based on image intensities of the plurality of polonies in cycle N+1, I(N+1).

23-28. (canceled)

29. The method of claim 22, further comprising:
making, by the processor, the base calls in the cycle N+1 using the corrected image intensities of the plurality of polonies in the cycle N+1, Ipc(N+1).

30. The method of claim 1, wherein obtaining the base calls in the cycle N based on the corrected image intensities of the plurality of polonies in the cycle N, Ipc(N) comprises:
making the base calls using the corrected image intensities of the plurality of polonies in the cycle N, Ipc(N), by identifying nucleotide bases corresponding to the plurality of polonies in the cycle.

31-34. (canceled)

35. The method of claim 1, wherein selecting the set of polonies from the plurality of polonies comprises:
for each channel of one or more channels:
selecting polonies that are called a base corresponding to the channel in the cycle N and called a different base in the cycle N−1;
selecting polonies that are called a base corresponding to the channel in the cycle N and called a different base in cycle N+1; or
a combination thereof.

36. The method of claim 1, further comprising:
fitting the image intensities of the set of selected polonies using one or two linear functions.

37. The method of claim 36, further comprising:
determining a slope for each of the one or two linear functions; and
generating the cycle N phasing coefficient, pN, the cycle N prephasing coefficient, ppN, or both based on the slope for each of the one or two linear functions.

38. The method of claim 1, further comprising:
for each channel of one or more channels, generating corrected normalized image intensities, Inorm_n(N), based on image intensities of polonies that are called a base corresponding to the channel; and
updating a normalized image intensity, Inorm(N), by using the corrected normalized image intensities, Inorm_n(N).

39. The method of claim 38, wherein the generating the corrected normalized image intensities, Inorm_n(N), based on the image intensities of the polonies that are called a base corresponding to the channel comprises:
determining a normalization factor using the image intensities of the polonies that are called the base corresponding to the channel.

40. The method of claim 39, wherein the normalization factor is an image intensity selected from a range of intensity among at least part of the polonies that are called the base corresponding to the channel.

41. (canceled)

42. The method of claim 38, further comprising:
updating the base calls of the cycle N based on the updated normalized image intensities, Inorm(N).

43-79. (canceled)

80. A method for phasing and prephasing correction of base calling in sequencing, comprising:
determining, by a processor, a cycle N−1 phasing coefficient, pN−1, a cycle N−1 prephasing coefficient, ppN−1, or both the cycle N−1 phasing coefficient and the cycle N−1 prephasing coefficient of a plurality of polonies of flow cell images in a cycle N−1;
determining, by the processor, corrected image intensities of the plurality of polonies in a cycle N, Ipc(N), based on the cycle N−1 phasing coefficient, pN−1, the cycle N−1 prephasing coefficient, ppN−1, and image intensities of the plurality of polonies in the cycle N, I(N); and
iterating, by a processor, until a stopping criteria is met, one or more of: determining, by the processor, corrected image intensities of the plurality of polonies in a cycle N+1, Ipc(N+1), based on:
the cycle N−1 phasing coefficient, pN−1; the cycle N−1 prephasing coefficient, ppN−1; and the image intensities of the plurality of polonies in the cycle N, I(N) in response to determining that a cycle N phasing coefficient, pN, and a cycle N prephasing coefficient, ppN, are not available; or
the cycle N phasing coefficient, pN; the cycle N prephasing coefficient, ppN; and the image intensities of the plurality of polonies in the cycle N, I(N), or image intensities of the plurality of polonies in the cycle N+1, I(N+1), in response to determine that the cycle N phasing coefficient, pN, and the cycle N prephasing coefficient, ppN, are available;
obtaining, by the processor, base calls in the cycle N based on the corrected image intensities of the plurality of polonies in the cycle N, Ipc(N);
selecting, by the processor, a set of polonies from the plurality of polonies based on the base calls in the cycle N;
determining, by the processor, the cycle N phasing coefficient, pN, and the cycle N prephasing coefficient, ppN, based on the image intensities of the set of selected polonies; and
updating, by the processor, the image intensities of the plurality of polonies in cycle N, I(N), using updated and corrected image intensities, Ipc_n(N), wherein Ipc_n(N) is obtained based on the cycle N phasing coefficient, pN, and the cycle N prephasing coefficient, ppN.

81-158. (canceled)

159. A method for phasing and prephasing correction in sequencing analysis, comprising:
generating, by a sequencing system, flow cell images by conducting one or more cycles of sequencing reactions of a plurality of nucleic acid template molecules immobilized on a support, wherein the flow cell image comprises a plurality of polonies corresponding to the plurality of nucleic acid template molecules;
selecting, by a processor, a subset of polonies from the plurality of polonies;
determining, by the processor, a cycle N phasing coefficient, pN, or a cycle N prephasing coefficient, ppN, that maximizes a quality or purity of image intensities of the set of selected polonies in the cycle N and is based on a penalty function;
updating, by the processor, image intensities of the plurality of polonies in cycle N, I(N), using updated and corrected image intensities, Ipc_n(N), wherein Ipc_n(N) is obtained based on the cycle N phasing coefficient, pN, the cycle N prephasing coefficient, ppN, or both; and
performing, by the processor, base calling of the plurality of polonies using the updated image intensities of the plurality of polonies, I(N), in cycle N.

160-237. (canceled)

238. The method of claim 1 further comprising:
generating, by a sequencing system, the flow cell images by conducting one or more cycles of sequencing reactions of a plurality of nucleic acid template molecules immobilized on a support, wherein the flow cell image comprises image intensities of the plurality of polonies corresponding to the plurality of nucleic acid template molecules in one or more samples, and wherein the one or more cycles comprises the cycle N and the cycle N−1.

239. The method of claim 1 further comprising:
generating, by a sequencing system, the flow cell images from one or more cellular sample(s) by conducting one or more cycles of sequencing reactions of a plurality of concatemer molecules of the one or more cellular sample(s) immobilized on a support, wherein the flow cell images comprise image intensities of the plurality of polonies corresponding to the plurality of concatemer molecules in the one or more samples, and wherein the one or more cycles comprises the cycle N and the cycle N−1.
